(19) United States
(12) Patent Application Publication
(10) Pub. No.: US 2022/0370545 A1
(43) Pub. Date: Nov. 24, 2022

(54) INTEGRIN RECEPTOR ALPHA V BETA 3 AND ITS LIGAND INVOLVED IN CHRONIC ITCH

(71) Applicant: North Carolina State University, Raleigh, NC (US)

(72) Inventors: Santosh K. Mishra, Cary (US); Thierry J M Olivry, Riga (LV); Joshua Wheeler, Raleigh (US)

(21) Appl. No.: 17/762,191

(22) PCT Filed: Sep. 18, 2020
(86) PCT No.: PCT/US2020/051438

§ 371 Date: Mar. 21, 2022

Related U.S. Application Data
(60) Provisional application No. 62/903,376, filed on Sep. 20, 2019.

(51) Int. Cl. A61K 38/08; A61K 31/4375; A61K 31/55; A61P 17/04; A61K 38/22; A61K 38/12

(52) U.S. Cl. CPC A61K 38/08; A61K 31/4375; A61K 31/55; A61P 17/04; A61K 38/22; A61K 38/12

(57) ABSTRACT

Methods of treating pruritis (e.g., associated with atopic dermatitis or psoriasis) are described. The methods can involve administering to a subject in need of treatment an antagonist of integrin αᵥβ₃. The antagonist can block periostin-integrin signalling. The antagonist can be, for example, an antibody, a peptide having a RGD or SVD sequence, a peptidomimetic, an amine salt, a phosphoric acid salt, or a small molecule.

### CROSS-REFERENCE TO RELATED APPLICATIONS

This application claims benefit of U.S. Provisional Patent Application Ser. No. 62/903,376, filed Sep. 20, 2019, which is herein incorporated by reference in its entirety.

### TECHNICAL FIELD

The presently disclosed subject matter relates to role of the integrin alpha V beta 3 (αvβ3) receptor and its endogenous ligand periostin in pruritis (itch). The presently disclosed subject matter further relates to the use of antagonists of integrin αvβ3 in treating and/or alleviating pruritis.

### BACKGROUND

Atopic dermatitis (AD)—also known as (atopic) eczema—is a common chronic allergic skin disease of humans and dogs with a prevalence estimated at up to 25% of children, a prevalence that depends upon the patient's age, ethnic background and geographical origin (Odhiambo et al., 2009). This condition often persists in adults (Abuabara et al., 2018). Atopic dermatitis has a high impact on the health of patients due to an elevated risk of co-morbidities, such as arthritis, asthma and allergic rhinitis (Eckert et al., 2017). As AD is also associated with a high prevalence of anxiety, depression, and sleep disorders, there is an ensuing reduced quality of life and work productivity (Eckert et al., 2018). As a result, AD leads to substantial healthcare expenses for both patients and society (Adamson, 2017; Eckert et al., 2017).

In addition to the classic erythema and eczematous lesions with a characteristic age-related distribution, AD is associated with a chronic recurrent itch that is often moderate-to-severe (Shahwan and Kimball, 2017; Weidinger and Novak, 2016). While recent findings have improved understanding of the itch sensation in mice and humans, additional methods are needed to successfully and completely treat this noxious sensation associated with many cutaneous and neurologic diseases (Carstens, 2008; Oaklander, 2011; Paus et al., 2006; Yosipovitch and Samuel, 2008).

Accordingly, there is an ongoing need in the art for new methods and compositions for treating or alleviating pruritis, particularly for chronic pruritis and other diseases or disorders associated with pruritis, such as psoriasis, eczema, multiple sclerosis, shingles, diabetes, insect bites, allergic reactions, burns, scars, and dry skin.

### SUMMARY

In some embodiments, the presently disclosed subject matter provides a method of treating or alleviating pruritus, optionally chronic pruritus, in a subject in need of treatment thereof, the method comprising administering to the subject an effective amount of an antagonist of integrin αᵥβ₃. In some embodiments, the pruritis is associated with one of atopic dermatitis or psoriasis.

In some embodiments, administration of the antagonist blocks periostin-integrin signaling. In some embodiments, the antagonist has a 50% inhibitory concentration (IC₅₀) for integrin αᵥβ₃ of about 50 nanomolar (nM) or less, optionally about 10 nM or less. In some embodiments, the antagonist is selective for integrin αᵥβ₃ compared to integrin αᵥβ₅. In some embodiments, the antagonist has a 50% inhibitor concentration (IC₅₀) for integrin αᵥβ₃ that is at least about 2 times lower than the antagonist's IC₅₀ for integrin αᵥβ₅, optionally at least about 5 times lower.

In some embodiments, the antagonist of integrin αᵥβ₃ is selected from the group comprising an antibody or a fragment thereof, a peptide comprising an RGD sequence, a peptide comprising an SDV sequence, a peptidomimetic, an amine salt, a phosphoric acid salt, and a small molecule antagonist of integrin αᵥβ₃. In some embodiments, the antagonist of integrin αᵥβ₃ is a peptide comprising an RGD sequence. In some embodiments, the peptide comprising an RGD sequence is a synthetic peptide. In some embodiments, the synthetic peptide is a cyclic peptide and/or a tetra- or pentapeptide. In some embodiments, in addition to the RGD sequence, the synthetic peptide comprises a residue based on a D-amino acid and/or a N-methylated residue. In some embodiments, the antagonist is cilengitide.

In some embodiments, the peptide comprising an RGD sequence is a naturally occurring peptide. In some embodiments, the peptide comprising an RGD sequence is a disintegrin. In some embodiments, the disintegrin is Echistatin.

In some embodiments, the antagonist is a peptide that comprises a SDV sequence. In some embodiments, the peptide is His-Ser-Asp-Val-His-Lys-NH₂ (SEQ ID NO: 2, P11).

In some embodiments, the antagonist is a peptidomimetic, wherein said peptidomimetic is a peptidomimetic of a peptide comprising an RGD sequence, optionally wherein said peptidomimetic comprises a monocyclic central phenyl ring, a monocyclic central heterocyclic ring, a bicyclic central ring, or an acyclic backbone. In some embodiments, the antagonist is a small molecule antagonist of integrin αᵥβ₃, optionally wherein the antagonist is (S)-3-(6-methoxypyridin-3-yl)-3-(2-oxo-3-(3-(5,6,7,8-tetrahydro-1,8-naphthyridin-2-yl)propyl)imid-azoleidin-1-yl)propanoic acid (L000845704) or (4S)-2,3,4,5-tetrahydro-8-[2-[6-(methylamino)-2-pyridinyl]ethyoxy]-3-oxo-2-(2,2,2-trifluoroethyl)-1H-2-benzazepine-4-acetic acid (SB273005).

In some embodiments, the presently disclosed subject matter provides a method of treating or alleviating pruritus, optionally chronic or acute pruritus, in a subject in need of treatment thereof, the method comprising administering to the subject an effective amount of cilengitide.

Accordingly, it is an object of the presently disclosed subject matter to provide a method of treating or alleviating pruritus.

An object of the presently disclosed subject matter having been stated hereinabove, and which is achieved in whole or in part by the presently disclosed subject matter, other objects will become evident as the description proceeds herein below.

### BRIEF DESCRIPTION OF THE DRAWINGS

FIG. 1A-1F: Periostin-induces a robust itch behavior mediated via somatosensory neurons. FIG. 1A is a graph showing the number of scratching bouts following an intradermal injection of vehicle (20 microliters (μl) phosphate buffered saline (PBS), black circles) or periostin (5 micrograms (μg)/20 μl, grey circles) into the dorsal neck of wild-type C57BL6J mice. Mice scratching bouts were recorded for 0-15 minutes and 15-30 minutes post injection. Periostin induced significant scratching bouts in 0-15 minutes compared to 15-30 minutes. There was no change in vehicle response between 0-15 minutes and 15-30 minutes, n=7-8 mice per group. FIG. 1B is a graph showing the duration of pruritus manifestations (DPM) following intradermal injection of periostin (25 μg/100 μl, grey circles) or vehicle (100 μl PBS, black circles) in the dorsal neck of dogs. DPM were measured for first 0-15 minutes and second 15-30 minutes. Periostin induced significant DPM in 0-15 minutes compared to 15-30 minutes. There was no change in vehicle response between 0-15 minutes and 15-30 minutes, n=8 dogs per group. FIG. 1C is a graph showing the number of scratches following subcutaneous injection of periostin (25 μg/100 μl, grey circles) or vehicle (100 μl PBS, black circles) in the thighs of monkeys. Number of scratches were recorded for 0-15 minutes and 15-30 minutes. Periostin induced significant scratching bouts in 0-15 minutes compared to 15-30 minutes. There was no change in vehicle response between 0-15 minutes, and 15-30 minutes, n=5 monkeys per group. FIG. 1D is a graph showing the number of scratching bouts observed within 30 minutes of an intradermal injection of vehicle (20 μl PBS, black circles) or periostin (5 μg/20 μl, grey circles) in the dorsal neck of control and mast cell-deficient mice. There was no change in periostin-induced scratching behaviors between control and mast cell-deficient mice, n=5-6 mice per group. FIG. 1E is a graph showing the number of scratching bouts observed within 30 minutes of an intradermal injection of vehicle (20 μl PBS, black circles) or periostin in the dorsal neck of control and B and T cell-deficient mice (5 μg/20 μl, grey circles). There was no change in periostin-induced scratching behaviors observed between control and B and T cell-deficient mice, n=6 mice per group. FIG. 1F is a graph showing the number of scratching bouts observed within 30 minutes following an intradermal injection of vehicle (20 μl PBS, black circles) or periostin (5 μg/20 μl, grey circles) in the dorsal neck of control and B, T, and NK cell-deficient mice. No change in periostin-induced scratching behaviors between control and mutant mice, n=6 mice per group. All data were presented as the mean±SEM in mice, dogs, and monkeys. One-tailed student's t-test was performed between two groups to determine significance, *p<0.05; **p<0.01.

FIGS. 2A-2F: Periostin evoked itch behavior but not pain directly through sensory neurons. FIG. 2A is a graph showing the number scratching bouts following an intradermal injection of vehicle (20 microliters (μl) phosphate buffered saline (PBS)), periostin (5 micrograms (μg)/20 μl), and histamine (100 μg/20 μl) into the cheek of C57BL6J mice, n=5-6 mice per group. FIG. 2B is a graph showing wiping response in wild-type mice. Wiping was counted for 10 minutes following an intradermal injection of periostin (5 μg/20 μl) and capsaicin as a positive control (1 μg/20 μl, square) into the cheek of C57BL6J mice, n=6 mice per group. FIG. 2C is a graph showing lack of wiping behavior following corneal application of periostin (5 μg/20 μl) or vehicle. Capsaicin application shows robust wiping behavior in wild-type mice but no response in TRPV1 KO mice. Wiping was measured for 1 minute, n=5 mice. FIG. 2D is a graph showing the percentage (%) of mast cells degranulated with different concentrations of dinitrophenyl (DNP). The results show maximum degranulation with 100 ng/ml DNP, n=3. FIG. 2E is a graph showing the percentage (%) of mast cells degranulated with different concentration of periostin. Results show no degranulation at various concentrations of periostin, n=3. FIG. 2F is a graph showing calcium influx measured in BMMC mast cells in response to vehicle, periostin, DNP, periostin plus DNP, and ionomycin, n=3. All data were presented as the mean±SEM. Significant differences between indicated groups were assessed using 1-way ANOVA with Dunn's multiple comparisons test for equal or more than three groups **p<0.001) and non-paired student's t-test was performed between two groups, *p<0.002.

FIGS. 3A-3E: Periostin integrin receptor subunits are expressed in dorsal root ganglia (DRG). FIG. 3A is a graph showing the expression of different subunits of integrin receptors relative to GAPDH in the DRG of 4 mice as measured using quantitative real time-polymerase chain reaction (qRT-PCR). FIG. 3B is a graph showing the expression of different subunits of integrin receptors relative to GAPDH in 3 dogs as measured using qRT-PCR. FIG. 3C is a graph showing the expression of different subunits of integrin receptors relative to GAPDH in 5 non-human primates as measured using qRT-PCR. FIG. 3D is a series of fluorescence microscope images showing the co-expression of the integrin receptor β3 subunit and SST-tdTomato (right). The fluorescence of integrin receptor β3 subunit alone is shown in the image at the top left, while the fluorescence of SST-tdTomato alone is shown in the image at the bottom left. The scale bar at the bottom right of the image on the right is 100 microns (μm). FIG. 3E is a graph showing the quantification of SST-tdTomato and integrin β3 in overlapping and non-overlapping populations, n=3 mice and an average of 3 sections per mouse. All data were presented as mean±SEM.

FIG. 4 is a graph showing concentration-dependent calcium influx to periostin. Periostin-evoked calcium response is concentration dependent. A dot point in the calcium imaging scatter plot represents one coverslip. All data were presented as mean±SEM, n=3 mice.

FIGS. 5A-5F: Periostin directly activates dorsal root ganglia (DRG) sensory neurons. FIG. 5A is a series of fluorescence microscope images of DRG neurons pre-incubated (45 minutes (min)) with the calcium dye Fura 2-AM (1 micromolar (μM)) and with calcium influx measured at the 340/380 wavelength. Arrows indicate cells responding to periostin (second image from left), AITC mustard (a TRPA1 agonist, second image from rigth) and capsaicin (a TRPV1 agonist, right). FIG. 5B is a graph showing the amplitude of cytosolic calcium (Ca²⁺) increase for a single region of interest taken every 100 milliseconds (ms). FIG. 5C is a graph showing the periostin-induced calcium response in DRG neurons treated with periostin (16 nanograms per microliter (ng/μl)), AITC mustard (100 μM) and capsaicin (1 μM), n≥6 mice. FIG. 5D is a graph showing that that were no neuronal calcium responses to periostin in the absence of extracellular calcium, n=3 mice. FIG. 5E is a graph showing that the periostin-induced calcium response is not affected by either the Gβγ blocker gallein (100 μM) or the phospholipase C inhibitor U73122 (1 μM). n=2 mice. FIG. 5F is a microscope image and graph showing somatostatin (SST)-positive medium-diameter neurons (fluorescence microscope image shown in left panel) after being patched and where the inward current was measured in response to periostin (10 micrograms per milliliter (μg/ml)) and 0.3 nanomoles per liter (nmol/l) capsaicin (graph on right). The graph shows that the SST-positive neurons produced an inward current to periostin suggesting the presence of the αvβ3 integrin receptor on SST-positive cells. Note: SST-positive neurons are a small subset of TRPV1-expressing neurons. The average current by periostin is 58.4 picoamperes (pA), total 8 neurons from 4 independent mice. A dot point in calcium imaging scatter plot represents one coverslip. All data were presented as mean±SEM and significance difference between two groups were determined by unpaired Student's t-test (** P≤0.01).

FIGS. 6A-6B: Pharmacological blockage of integrin receptors inhibits both calcium influx and periostin-evoked itch behavior. FIG. 6A is a graph showing the pharmacological blockage of integrin receptors (as a percentage of neurons blocked) by cilengitide (100 nanomolar (nM)), a non-specific blocker for integrin receptors αvβ3 and αvβ5, inhibits the periostin-induced calcium response, but has no effect on the mustard or capsaicin-induced responses. n=4-5 mice and each data point represents one coverslip. FIG. 6B is a graph showing the number of bouts of scratching observed after injections of periostin (5 micrograms (μg)/20 microliters (μl)) following the co-administration of cilengitide by different routes (100 nM) in mice. n=5-6 mice each group. All data were presented as mean±SEM and significance difference between two groups were determined by unpaired Student's t-test (*P≤0.05, ** P≤0.01).

FIG. 7 is a pair of fluorescence microscopy images showing integrin receptor subunit αv and β5 expression in the dorsal root ganglia (DRG). The images show the results of double immunohistochemistry (IHC) assays that illustrate the expression of the integrin receptor αv (left) and β5 subunit (right)) in the majority of sensory neurons and that somatostatin (SST)-positive neurons are small subset.

FIGS. 8A-8J: The periostin-mediated itch behavior implicates the integrin receptor αvβ3 in transient receptor potential cation channel subfamily V member 1 (TRPV1)-expressing neurons. FIG. 8A is a pair of fluorescence microscopy images of an immunohistochemistry showing the elimination of β3 in TRPV1-cre::β3−/− mice compared to either TRPV1-cre (right image) or β3^{f/f} alone as control (left image). The arrowheads indicate β3-positive cells. The scale bar in the bottom right of each image represents 75 microns (μm). FIG. 8B is a graph showing the quantification of β3-immunopositive cells of dorsal root ganglias (DRGs) from the TRPV1-cre:β3−/− mice, demonstrating a significant reduction compared to control littermates (β3^{f/f}), n=3-4 mice and an average of 3 sections from each mice was quantified. FIG. 8C is a graph showing that the periostin-mediated calcium response (measured as a percentage of neurons) is reduced in TRPV1-cre:β3−/− mice. There was no change in the capsaicin induced calcium influx, n=3-4 mice and each data point represents one coverslip. FIG. 8D is a graph showing itch behavior (measured as the number of bouts of scratching per 30 minutes) following the injection in the nape of the neck of vehicle (phosphate buffered saline (PBS)) or periostin in control and mutant (TRPV1-cre:β3−/−) mice. Periostin-induced itch was significantly reduced in mutant mice as compared to control littermates, n=5-6 mice per group. FIG. 8E is a graph showing that the itch response (measured as the number of bouts of scratching per 30 minutes) to intradermal injection of histamine in control littermates and mutant (TRPV1-cre:β3−/−) mice remained normal, n=6 mice per group. FIG. 8F is a graph showing that the itch response (measured as the number of bouts of scratching per 30 minutes) to intradermal injection of chloroquine in control and TRPV1-cre::β3−/− mice remained normal, n=5 mice per group. FIG. 8G is a graph showing the results (measured as withdrawal latency in seconds (s)) of Hargreaves assays between control and mutant (TRPV1-cre:β3−/− mice) littermates. Both control and mutant mice had a normal withdrawal latency, n=5-6 per group. FIG. 8H is a graph showing the results (measured as withdrawal latency in seconds (s)) of dry ice cold assays between control and mutant (TRPV1-cre:β3−/−mice) littermates. Both control and mutant mice showed normal behavior responses, n=5-6 per group. FIG. 8I is a graph showing the results (measured as force (in grams)) of touch (von-Frey) tests between control and mutant (TRPV1-cre::β3−/− mice) littermates. Both control and mutant mice demonstrated normal behavior responses, n=5-6 per group. FIG. 8J is a graph showing the results (measured as time spent (in seconds (s)) of a rotarod test to show motor deficits between control and mutant (TRPV1-cre::β3−/−) littermates. Both control and mutant mice had normal behavior responses, n=5-6 per group. All data were presented as mean±SEM. one way ANOVA Dunn's test was performed for equal or more than three groups (** p<0.001), and unpaired Student's t-test was performed to determine significance (*p≤0.01).

FIGS. 9A-9E: The periostin-mediated calcium influx involves the downstream activation of transient receptor potential (TRP) channels and periostin-evoked itch dependent on TRP channels and neuropeptide NPPB. FIG. 9A is a graph showing the periostin-induced calcium response (measured as percentage of neurons) in transient receptor potential cation channel subfamily V member 1 (TRPV1)-, transient receptor potential cation channel subfamily A member 1 (TRPA1)-, and double-KO mice. Periostin-induced calcium response was inhibited in all three groups compared to control mice. The percentage of neurons that responded to periostin was normalized with potassium chloride (KCl, 1 millimolar (mM)), n=3-4 mice and each data point represents one coverslip. FIG. 9B is a graph showing the itch behavior (measured as number of bouts of scratching) following intradermal injection of periostin in the dorsal neck of control littermates and TRPV1 knockout (KO) mice. A significant reduction in itch behavior was observed in TRPV1 KO mice compared to control littermates, n=5-7 mice per group. FIG. 9C is a graph showing reduction in itch behavior (measured as the number of bouts of scratching) in TRPA1 KO mice compared to control littermates, n=6 mice per group. FIG. 9D is a graph showing a reduction in itch behavior (measured as the number of bouts of scratching) in double KO (TRPV1+TRPA1) mice compared to control littermates, n=6 mice per group. FIG. 9E is a graph showing a reduction in itch behavior (measured as the number of bouts of scratching) in NPPB KO mice compared to control littermates, n=6-7 mice per group. All data were presented as mean±SEM, Significance was determined by unpaired Student's t-Test (*p=0.01, **p≤0.008).

FIGS. 10A-10D: Expression of thymic stromal lymphopoietin receptor (TSLPR)/interleukin-7-receptor subunit alpha (IL7Rα) receptor complex and translocation and detection of Signal Transducer and Activation of Transcription (STAT) phosphorylation by Western blotting. FIG. 10A is a set of representative traces of calcium influx of thymic stromal lymphopoietin (TSLP, 2 nanograms per milliliter (ng/ml), left) and ionomycin (10 micromolar (μM), right) on mouse keratinocyte cell line that were cultured for 24 hrs, n=3 coverslips. FIG. 10B is an image of a skin immunoblot showing the expression of TSLPR in a mouse keratinocytes cell line (left) and skin (right). FIG. 10C is a series of fluorescence images showing that TSLP-mediates the translocation of STAT3 in mouse keratinocytes. There was no change in the expression of whole STAT3 (B and F, images in column second from left), whereas the activated form, phospho-STAT3, transported to the nucleus in response to 10 ng/ml TSLP (G compared to C [phosphate buffered saline (PBS) control], images in column second from right). Outlined images in C and G are further magnified (D and H, images in column on right). Data were repeated three time with similar results. FIG. 10D is an image of a representative immunoblot of both phosphorylated and non-phosphorylated STATS and STAT6, which shows no change in in response to TSLP (10 ng/ml) compared to vehicle (PBS) treated. Samples were probed with antibodies against pSTATS, pSTAT6, STATS, STAT6, and glyceraldehyde 3-phosphate dehydrogenase (GAPDH).

FIGS. 11A-11K: Keratinocytes secrete periostin in response to thymic stromal lymphopoietin (TSLP) stimulus and the activation of the Janus Kinase/Signal Transducer and Activator of Transcription (JAK/STAT) pathway. FIG. 11A is a graph showing that TSLP provokes the release of periostin (measured in picograms per milliliter (pg/ml) from Balb/MK2 mouse keratinocytes. Equal number of cells (50,000 cells/well) were plated and treated with 1 and 10 nanograms per milliliter (ng/ml) TSLP and supernatants were analyzed after 24 hours (hrs) by enzyme-linked immunosorbent assay (ELISA), n=3 independent treatments at each concentration. FIG. 11B is a graph showing the inhibitory effect of the JAK2 inhibitor, SD 1008, and the STAT3 inhibitor, niclosamide, on TSLP-induced periostin production and release (measured in pg/ml) by mouse BALB/MK2 keratinocytes. Cells were pre-treated for 4 hrs with SD 1008 or Niclosamide, and then stimulated with 10 ng/ml TSLP and periostin release measured by ELISA after 24 hrs, n=3 independent treatments at each concentration. FIG. 11C is an image of representative immunoblots of both periostin and phospho-STAT3 showed that both were upregulated in mouse skin in response to TSLP (10 ng/ml) compared to vehicle-(phosphate buffered saline (PBS)) treated. Samples were probed with antibodies against periostin, calnexin, pSTAT3, and totalSTAT3. FIG. 11D is a graph showing the quantification of periostin normalized with calnexin, n=3 mice. FIG. 11E is a graph showing the quantification of pSTAT3 normalized with totalSTAT3, n=3 mice. FIG. 11F is a photographic image of mice following topical application of vehicle (ethanol, left mouse) and 4 nanomoles (nmol) of vitamin-D analog calcipotriol (MC903, right mouse) each day up to 7 days. MC903-induced erythema and scaling compared to ethanol-treated mice. The image was taken on day 7. FIG. 11G is an image of a representative immunoblot of periostin and calnexin as a control, performed using skin lysates of vehicle and MC903-treated mice on Day 7. MC903 (4 nmol) and ethanol were applied onto the neck of C57BL6 mice. FIG. 11H is a graph showing the quantification of periostin-normalized with calnexin A significant increase in periostin production in mice treated with MC903 compared to ethanol, n=4 mice. FIG. 11I is a graph showing skin thickness measured (in microns (μm) each day. MC903-induced skin thickness compared to vehicle treated mice but there were no change in skin thickness seen between control (black) and TRPV1-cre:β3−/− mice (grey) littermates, n=4-5 mice. FIG. 11J is a graph showing that MC903-induced scratching bouts (measured over 30 minutes) was day-dependent and was significantly increased at Day 7 when compared to Day 1. TRPV1-cre::β3−/− mice (grey) littermates showed a significant reduction in scratching bouts compared to control littermates (black), n=9 mice. All data were presented as mean±SEM, one-way and two-way ANOVA Dunn's test were performed as appropriate ((*p≤0.05, **p≤0.01), and Student's t-test between two groups (*p≤0.05, **p≤0.01). FIG. 11K is a schematic diagram showing how TSLP, MC903 and house dust mites (HDM) induce the release of periostin in the skin. The secreted periostin then binds to the integrin receptor αvβ3 on dorsal root ganglia (DRG) sensory neurons, activates downstream transient receptor potential (TRP) channels (TRPV1 and TRPA1) to later release neurotransmitters/neuropeptides NPPB in the spinal cord and activate one or more interneurons to eventually induce itch.

FIGS. 12A-12B: House dust mite (HDM)-induced periostin in the skin and itch in atopic dermatitis (AD) mouse model. FIG. 12A is a graph showing the periostin production (measured in picograms per milligram (pg/mg)) in NC/Nga mice topically treated with Dermatophagoides farinae HDM (10 milligrams per milliliter (mg/ml)). Enzyme-linked immunosorbent assay (ELISA) was performed on the skin tissue homogenates to measure periostin and compared that to mineral oil. A significant decrease in periostin production in mice treated with glucocorticoids betamethasone compared to vehicle treated group, n=8 mice. FIG. 12B is a graph showing increase in scratching bouts observed in NC/Nga mice applied topically with allergen HDM (10 mg/ml) compared to mineral oil. The scratching behavior was significantly reduced in mice treated with glucocorticoids betamethasone compared to vehicle treated group, n=8 mice. All data were represented in mean±SEM, Significant difference between more than two groups were performed using one-way ANOVA with Dunn's test (***P<0.001).

FIG. 13 is a pair of representative images of immunohistochemistry (IHC) assays from the dorsal nape of the neck skin of three mice injected with thymic stromal lymphopoietin (TSLP, 10 nanograms per milliliter (ng/ml), image on right) revealing increased expression of periostin in keratinocytes (epidermis is shown in white box) compared to mice injected with vehicle (phosphate buffered saline (PBS), image on left). The tissues were imaged 8 hours after TSLP and vehicle injections. White arrow shows expression of periostin in dermal layer. The scale bar in the lower right of the image on the right represents 50 microns (μm).

FIGS. 14A-14B: Integrin blocker inhibits histamine and chloroquine (CQ)-induced itch. FIG. 14A is a graph showing the number of scratching bouts measured for 30 minutes in mice following injection of cilengitide 10 minutes prior to histamine injection. FIG. 14B is a graph showing the number of scratching bouts measured in 30 minutes in mice following injection of cilengitide 10 minutes prior to CQ injection. Data are presented as mean±SEM and significance difference between two groups were determined by unpaired Student's t-test (*p≤0.05, **p≤0.00).

FIG. 15 is a graph showing the effect on periostin-induced calcium response in dorsal root ganglia (DRG) neurons following pre-treatment with P11 blocker. There is no change in capsaicin response (1 micromolar (μM)). A total of 389-418 neurons from n≥2 mice. A dot point in calcium imaging scatter plot represents one coverslip. All data were presented as mean±SEM and significance difference between two groups were determined by an unpaired Student's t-test (*p≤0.02).

FIG. 16 is a graph showing the effect on periostin-induced calcium response in dorsal root ganglia (DRG) neurons following treatment with antibody blocker LM206. Capsaicin response (1 micromolar (μM)) remains unaffected with and without LM206, a total of 300-350 neurons from n≥2 mice. A dot point in calcium imaging scatter plot represents one coverslip. All data were presented as mean±SEM and significant difference between two groups were determined by an unpaired Student's t-test (p≤0.05) for significant difference and anything above non-significant (ns).

FIG. 17 is a graph showing the effect on periostin-induced calcium response in dorsal root ganglia (DRG) neurons following treatment with the natural peptide inhibitor Echistatin. Capsaicin response (1 micromolar (μM)) remains unaffected in presence of Echistatin. A total of 220-299 neurons from n≥2 mice. A dot point in calcium imaging scatter plot represents one coverslip. All data were presented as mean±SEM and significance difference between two groups were determined by an unpaired Student's t-test (*p≤0.01).

FIG. 18 is a graph showing the effect on periostin-induced calcium influx response following treatment with the small molecular inhibitor MK-0429 (80 nanomolar (nM) (black squares) or 160 nM (black triangles)). High dose of MK-0429 significantly reduced the periostin-induced calcium influx. Capsaicin response (1 micromolar (μM)) remains unaffected in the presence of MK-0429 inhibitor. A total of 285-365 neurons from n≥2 mice were tested. A dot point in calcium imaging scatter plot represents one coverslip. All data were presented as mean±SEM and significant difference between the two groups was determined by an unpaired Student's t-test (P≤0.05) for significance and anything above non-significant (ns).

FIG. 19 is a graph showing the effect on periostin-induced calcium influx response following treatment with the small molecule inhibitor SB273005 antagonist (11 nanomolar (nM) (black squares) or 20 nM (black triangle)). High dose of SB273005 significantly reduced the periostin-induced calcium influx. Capsaicin response (1 micromolar (μM)) remains unaffected in the presence of inhibitors. A total of 398-422 neurons from n≥2 mice were tested. A dot point in calcium imaging scatter plot represents one coverslip. All data were presented as mean±SEM, and significant difference between two groups were determined by an unpaired Student's t-test (P≤0.05) for significance and anything above non-significant (ns).

FIG. 20 is a graph showing an MC903-induced AD mouse model that causes spontaneous scratching behavior at Day 10. Cilengitide injected through tail i.v. injection (100 nanomolar (nM)) significantly inhibited the spontaneous itching in mice. All data were presented as mean±SEM, and significant difference between two groups were determined by an unpaired Student's t-test (P≤0.05) for significance).

### DETAILED DESCRIPTION

The presently disclosed subject matter will now be described more fully. The presently disclosed subject matter can, however, be embodied in different forms and should not be construed as limited to the embodiments set forth herein below and in the accompanying Examples. Rather, these embodiments are provided so that this disclosure will be thorough and complete, and will fully convey the scope of the embodiments to those skilled in the art.

All references listed herein, including but not limited to all patents, patent applications and publications thereof, and scientific journal articles, are incorporated herein by reference in their entireties to the extent that they supplement, explain, provide a background for, or teach methodology, techniques, and/or compositions employed herein.

### I. Definitions

While the following terms are believed to be well understood by one of ordinary skill in the art, the following definitions are set forth to facilitate explanation of the presently disclosed subject matter.

Unless defined otherwise, all technical and scientific terms used herein have the same meaning as commonly understood to one of ordinary skill in the art to which the presently disclosed subject matter belongs.

Following long-standing patent law convention, the terms “a,” “an,” and “the” refer to “one or more” when used in this application, including the claims.

The term “and/or” when used in describing two or more items or conditions, refers to situations where all named items or conditions are present or applicable, or to situations wherein only one (or less than all) of the items or conditions is present or applicable.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” can mean at least a second or more.

The term “comprising,” which is synonymous with “including,” “containing,” or “characterized by” is inclusive or open-ended and does not exclude additional, unrecited elements or method steps. “Comprising” is a term of art used in claim language, which means that the named elements are essential, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising,” “consisting of,” and “consisting essentially of,” where one of these three terms is used herein, the presently disclosed and claimed subject matter can include the use of either of the other two terms.

Unless otherwise indicated, all numbers expressing quantities of size, temperature, time, weight, volume, concentration, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about,” when referring to a value is meant to encompass variations of in one example ±20% or ±10%, in another example ±5%, in another example ±1%, and in still another example ±0.1% from the specified amount, as such variations are appropriate to perform the disclosed methods.

Numerical ranges recited herein by endpoints include all numbers and fractions subsumed within that range (e.g. 1 to 5 includes, but is not limited to, 1, 1.5, 2, 2.75, 3, 3.90, 4, and 5).

In some embodiments, “treatment” or “treating” refers to an amelioration of disease or disorder, or at least one discernible symptom thereof, such as an itch sensation. “Treatment” or “treating” can refer to reducing or eliminating an itch sensation.

The term “alleviating” as used herein refers to reducing a symptom of a disease or disorder.

The term “peptide” as used herein refers to a polymer of amino acid residues, wherein the polymer can optionally further contain a moiety or moieties that do not consist of amino acid residues (e.g., an alkyl group, an aralkyl group, an aryl group, a protecting group, or a synthetic polymer, such as, but not limited to a biocompatible polymer). The term applies to amino acid polymers in which one or more amino acid residue is an artificial chemical mimetic of a corresponding naturally occurring amino acid, as well as to naturally occurring amino acid polymers and non-naturally occurring amino acid polymer.

The terms “peptidyl” and “peptidyl moiety” refer to a monovalent peptide or peptide derivative

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs are compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics are chemical compounds that have a structure that is different from the general chemical structure of an amino acid, but that functions in a manner similar to a naturally occurring amino acid.

The terms “residue” and “amino acid residue” as used herein refers to a divalent amino acid or derivative thereof. In some embodiments, the term “amino acid residue” refers to the group —NHC(R′)C(═O)—″, wherein R′ is an amino acid side chain or protected derivative thereof.

The term “peptidomimetic” refers to a compound that resembles a peptide, structurally and/or functionally, but which includes at least one non-peptidyl moiety. In some embodiments, the peptidomimetic comprises a backbone moiety, such as a cyclic or heterocyclic ring, that is not present in a natural peptide, but which mimics an amide bond.

The term “small molecule” as used herein generally refers to a synthetic or naturally occurring compound having a molecular weight of about 900 daltons or less.

As used herein, the term “conservative amino acid substitution” is defined herein as an amino acid exchange within one of the five groups summarized in Table 1, below.

TABLE 1
Conservative Amino Acid Substitutions

| Group | Characteristics | Amino Acids |
|---|---|---|
| A. | Small aliphatic, nonpolar or | Ala, Ser, Thr, Pro, Gly |
| | slightly polar residues | |
| B. | Polar, negatively charged residues | Asp, Asn, Glu, Gln |
| | and their amides | |
| C. | Polar, positively charged residues | His, Arg, Lys |
| D. | Large, aliphatic, nonpolar residues | Met Leu, Ile, Val, Cys |
| E. | Large, aromatic residues | Phe, Tyr, Trp |

### II. General Considerations

### II.A. Atopic Itch

The mechanism of atopic itch is complex, as it begins with the cutaneous release of a myriad of pruritogenic mediators including histamine, neurotrophins, eicosanoids, proteases, and cytokines (reviewed in (Bautista et al., 2014; Mollanazar et al., 2016; Storan et al., 2015; Voisin et al., 2017). Notable pruritus-inducing or -sensitizing cytokines are those typical of type 2 (Th2) immune reactions, such as interleukin (IL)-4 and -13 (Cevikbas et al., 2014; Dillon et al., 2004; Oetjen et al., 2017) and thymic stromal lymphopoietin (TSLP) (Wilson et al., 2013).

Pruritogenic mediators secreted in the skin will generally bind to their respective receptors located on neurites of peripheral somatosensory neurons with a cell body located in the dorsal root ganglia (DRG) (Bautista et al., 2014; Han and Dong, 2014; Mollanazar et al., 2016). These pruritogens activate either G-protein coupled (GPCRs) (Nguyen et al., 2017; Wilson et al., 2011a; Han et al., 2006; Imamachi et al., 2009), interleukin (Cevikbas et al., 2014) or toll-like receptors (Liu and Ji, 2014) as well as transient receptor potential (TRP) channels on DRG sensory neurons to begin the transduction of the itch signal to the central neural system (Imamachi et al., 2009; Kittaka and Tominaga, 2017; Shim et al., 2007). Similarly, sensory neurons appear to co-opt classic immune pathways to mediate chronic itch, which is dependent on neuronal IL-4Rα and JAK1 signaling (Oetjen et al., 2017). The next step in the itch propagation is the release of neurotransmitters from the primary afferents in the spinal cord. Several neurotransmitters have been characterized that either excite and/or inhibit itch neurotransmission (Ma, 2014; Mishra and Hoon, 2013, 2015). Among them, the B natriuretic peptide (BNP), also known as natriuretic polypeptide B (NPPB) was identified and found to be expressed by the small subset of neurons in the DRG which is involved in chemical-induced itch. Thus, NPPB-expressing DRG neurons are believed to be the (inflammatory) ‘itch neurons’ in the DRG (Mishra and Hoon, 2013). Recently, somatostatin (SST) was also shown to be expressed in itch-transmitting DRG neurons, of which nearly all also secrete NPPB (Huang et al., 2018).

II.B. Periostin and Integrin αvβ3

Periostin is a fasciclin extracellular matrix protein that exerts its function after binding to cell-surface receptors of the integrin family that include αvβ3 and αvβ5 (Izuhara et al., 2017). After stimulation with various stimuli including TGFβ and the Th2 cytokines IL-4 and IL-13, periostin is secreted by at least three types of cells including fibroblasts, epithelial and endothelial cells (Izuhara et al., 2017; Masuoka et al., 2012). Because of its fibroblast-rich environment, periostin is highly expressed in the skin, where its strongest immunostaining is found at the dermoepidermal junction (Yamaguchi, 2014). Periostin appears to be critical to the granulation and remodeling stages of cutaneous wound healing, as it promotes the differentiation and migration of fibroblasts and the proliferation of keratinocytes (Yamaguchi, 2014). Furthermore, periostin was recently found to be expressed in several disease states in which fibrosis is observed, for example, hypertrophic scars, bronchial asthma, pulmonary and systemic fibrosis and psoriasis (Yamaguchi, 2014). Periostin is also produced in the skin of both humans (Kou et al., 2014) and dogs with spontaneous AD (Merryman-Simpson et al., 2008; Mineshige et al., 2015). This fibrogenic cytokine is upregulated after epicutaneous allergen challenges in mouse (Masuoka et al., 2012; Shiraishi et al., 2012) and dog models of AD (Olivry et al., 2016); in the latter, it is transcribed late after an epicutaneous allergen provocation (Olivry et al., 2016). In humans with AD, serum levels of periostin not only correlate with disease activity, but they appear to reflect the chronicity of the disease, as its levels are highest when skin lichenification (thickening) is present (Kou et al., 2014). As periostin induces the secretion of the Th2 cytokine-promoting TSLP by keratinocytes (Shiraishi et al., 2012), an amplification loop involving periostin (i.e. periostin→TSLP→Th2 cytokines→periostin) is a mechanism suspected to lead to the dermal remodeling and epidermal hyperplasia typical of chronic AD (Masuoka et al., 2012; Shiraishi et al., 2012; Takahashi et al., 2016).

Because of the likely role of periostin in the pathogenesis of chronic skin lesions of AD, the studies underlying the presently disclosed subject matter were based on the hypothesis that periostin is also able to induce pruritus in AD. Described herein, the pruritogenic potential of periostin when injected into the skin of three mammalian species is characterized. As described further in the examples, it is confirmed, using molecular, pharmacological, cellular, and physiological assays, that periostin can directly activate the sensory neurons via integrin αvβ3, whose removal or inhibition reduces the pruritogenic effect of its ligand. Further, it is shown herein that TSLP, MC903, and house dust mites (HDM) all induce the expression and secretion of periostin in keratinocytes, thereby confirming the possibility of a TSLP-induced periostin release by epidermal cells which not only induces chronic inflammation, but also itch.

More particularly, the presently disclosed subject matter is based, in part, on the role of a subtype of integrin receptor expressed on the DRG sensory neurons in itch. The role of this integrin receptor in sensory itch detection and transmission has not been previously described. It is shown herein how the endogenous ligand from the receptor is upregulated and acts as an itch mediator and induces itch in mice. Using pharmacological, molecular, and conditional knockout mice data, it is shown how the neural circuit is involved in chronic allergic itch. Interference with this circuit can be used in the treatment or alleviation of chronic or acute itch, e.g., related to atopic dermatitis, psoriasis, and other skin and neurological diseases.

### III. Methods of Treating or Alleviating Pruritus

Accordingly, in some embodiments, the presently disclosed subject matter provides a method of treating or alleviating pruritus (i.e., itch), in a subject in need of treatment thereof, the method comprising administering to the subject an effective amount of an antagonist of integrin αᵥβ₃. In some embodiments, the pruritis can be associated with one of AD, psoriasis or another allergic and/or inflammatory skin diseases or a neurological disease. Thus, for example, the pruritis can be related to AD, psoriasis, eczema (dermatitis), burns, scars, dry skin, insect bites, scabies, hives, an allergic reaction, multiple sclerosis, diabetes, shingles, etc. In some embodiments, the pruritis is chronic pruritis (i.e., pruritis lasting more than about six weeks). In some embodiments, the pruritis is acute pruritis. The pruritis can be localized or more general. In some embodiments, provided is a method of treating or alleviating pruritus, optionally chronic or acute pruritus, in a subject in need of treatment thereof, the method comprising administering to the subject an effective amount of cilengitide.

In some embodiments, administration of the antagonist blocks periostin-integrin signaling. In some embodiments, the pruritis is associated with upregulated periostin. In some embodiments, the antagonist has a 50% inhibitory concentration (IC₅₀) for integrin αᵥβ₃ of about 150 nanomolar (nM) or less, about 125 nM or less, about 100 nM or less, about 75 nM or less, about 50 nanomolar or less, about 40 nM or less, about 30 nM or less, about 25 nM or less, about 20 nM or less, about 15 nM or less or about 10 nM or less. The IC₅₀ can be determined, for example, by any suitable assay known in the art for determining the IC₅₀ of a molecule to the integrin αᵥβ₃ receptor, e.g., an αᵥβ₃ binding assay, a kistrin-αᵥβ₃ inhibition assay, a αᵥβ₃ displacement assay, a vitronectin-αᵥβ₃ binding assay, etc. In some embodiments, the antagonist is a dual antagonist for αᵥβ₃ and αᵥβ₅ integrin receptors. In some embodiments, the antagonist is selective for integrin αᵥβ₃ compared to integrin αᵥβ₅ (e.g., wherein the IC₅₀ of the antagonist for αᵥβ₃ is at least 2 times smaller, at least about 5 times smaller, or at least about 10 times smaller than the IC₅₀ of the antagonist for αᵥβ₅). In some embodiments, the antagonist is a selective monoclonal antibody that blocks the receptor functions, thereby blocking itch. In some embodiments, the monoclonal antibody is species specific for its binding and receptor blocking functions.

Various antagonists for the integrin αᵥβ₃ receptor are known in the art. See, for example, Hsu et al., Recent Patents on Anticancer Drug Discovery, 2007, 2, 143-160; and Millard, et al., Theranostics, 2011, 1, 154-188; Reinmuth et al., Cancer Research, 2003, 63, 2079-2087; and Wang et al., Experimental and Therapeutic Medicine, 2014, 7, 1677-1682. See also, U.S. Patent Application Publication No. 2018/0344803, which is incorporated hereby by reference in its entirety. In some embodiments, the antagonist of integrin αᵥβ₃is selected from the group comprising an antibody or a fragment thereof, a peptide comprising an RGD sequence, a peptide comprising a SDV sequence, a peptidomimetic, an amine salt, a phosphoric acid salt, and a small molecule antagonist of integrin αᵥβ₃.

For example, antibodies that are antagonists of integrin αᵥβ₃ include anti-αvβ₃ monoclonal antibodies, humanized monoclonal antibodies, and chimeric antibodies. Representative antibody antagonists include, but are not limited to, LM609, Vitaxin I (MEDI-523), Abegrin (MEDI-522), CNTO 95, c7E3, and 17E6. In some embodiments, the selection of the antibody antagonist can be based on the species specificity of the antibody (e.g., when blocking itch in a particular species, the antibody can be an anti-αᵥβ₃ monoclonal antibody that is directed against that species' integrin αᵥβ₃). LM609, Vitaxin I (Abegrin MEDI-523), CNTO95, c7E3, and 17E6 are all human monoclonal antibodies. See Trikha et al., Int J Cancer 2004 Jun. 20;110(3):326-35; Faulds et al., Drugs, 1994 October;48(4):583-98; and Mitjans et al., 1995, J Cell Sci. 108 (Pt 8):2825-38. The antibodies have no reactivity with mouse integrin receptors. Based on the specificity information these antibodies may not block αVβ3 and αVβ5 receptors function in mouse DRG neurons but still have application in human and animal itch by blocking these receptors. In some embodiments, antibody testing is performed in either immortalized human DRG cells (cell line 50B11, Chen et al., J Peripher Nery Syst. 2007 June;12(2):121-30) or neuronal cell lines as model DRG neurons (ND7/23; SIGMA catalog #92090903-CDNA-20UL) to test the inhibitory role of the human antibody.

In some embodiments, the antagonist is a peptide comprising an arginine-glycine-asparagine (RGD) or a serine-asparagine-valine (SDV) sequence. In some embodiments, the peptide is a synthetic peptide. In some embodiments, the peptide is a cyclic peptide (e.g., a cyclic azapeptide). In some embodiments, the peptide is a synthetic tetra- or pentapeptide. In some embodiments, the peptide includes a residue based on a D-amino acid in addition to a RGD sequence and/or a N-methylated residue in addition to the RGD sequence. In some embodiments, the peptide is Cilengitide, i.e., cyclo RGDf-n(Me)V (SEQ ID NO:1), where f indicates D-Phe and the peptide bond between f and V is methylated). In some embodiments, the peptide comprising a RGD sequence is a naturally occurring peptide, such as a disintegrin. In some embodiments, the peptide is Echistatin. In some embodiments, the antagonist is a peptide that comprises a SDV sequence. In some embodiments, the peptide is P11, i.e., His-Ser-Asp-Val-His-Lys-NH₂ (SEQ ID NO:2). Peptide antagonists of αᵥβ₃, including those that do not include a RGD sequence, are also described, for example, in U.S. Pat. Nos. 5,753,230; 5,849,865; WO 9901472; WO 9910371; EP 1077218; U.S. Patent Application Publication No. 2004/0259798; and U.S. Pat. No. 5,780,426; each of which is incorporated herein by reference in its entirety.

In some embodiments, the antagonist is a peptidomimetic, e.g., a peptidomimetic of a peptide comprising an RGD sequence. In some embodiments, the peptidomimetics comprise small peptide-like chains containing natural and synthetic amino acids. In some embodiments, the peptidomimetic can be categorized by its backbone configuration, which can confer selective advantages for integrin binding and adherence. In some embodiments, the backbone of the peptidomimetic comprises a central monocyclic phenyl ring, a central monocyclic heterocyclic ring (e.g., a thiophene, a oxazole, a thiazole, a pyrrole, a pyrazinone, a pyridine, a pyrrolidinone, isoxazoline, an isoxazole, a thiodiazole, or an oxadiazole), or a central bicyclic ring (e.g., a naphthylene, a benzotriazole, a benzoimidazole, a dihydroisoquinolone, a benzazepine, a benzocycloheptanone, a benzocycloheptene, a benzocycloheptene, a benzodiazepine, or a quinolizinone). In some embodiments, the peptidomimetic has an acyclic backbone. Peptidomimetic antagonists of αvβ3 are also described, for example, in U.S. Pat. Nos. 5,929,120; 5,741,796; WO 9831359; WO 9800395; EP 0820991; WO 9932457; WO 9937621; WO 9945927; WO 0114338; U.S. Pat. No. 6,028,223; WO 9944994; WO 0187840; WO 0047564; WO 0031046; WO 0031044; U.S. Patent Application Publication No. 2004/0018192; WO 9952879; WO 9938849; WO 9952872; WO 0006169; WO 0003973; WO 015753; WO 0047552; WO 9412181; WO 0035862; WO 0024724; WO 9952896; U.S. Pat. Nos. 5,773,644; 5,852,210; 5,952,381; 5,773,646; 5,843,906; 5,710,159; 5,760,029; WO 9926945; WO 9959992; WO 0000486; WO 0075129; EP 0928793; EP 0796855; WO 9930713; WO 9930709; U.S. Pat. No. 5,981,546; WO 0009503; U.S. Pat. Nos. 6,017,926; 5,776,937; WO 0124797; WO 0144230; WO 0123376; WO 0078317; WO 0061551; WO 0031067; WO 0144194; U.S. Pat. Nos. 5,925,655; 5,919,792; 5,760,028; WO 9823608; WO 0031070; WO 0017197; WO 9835949; EP 0853084; WO 0158893; WO 9933798; WO 0126212; WO 0240505; U.S. Patent Application Publication No. 2004/0019035; EP 0854140; WO 9600574; WO 9814192; WO 9915170; WO 9915178; WO 9815278; WO 995107; U.S. Pat. No. 6,008,213; WO 9830542; WO 9915508; WO 0179172; U.S. Pat. No. 6,008,214; WO 9906049; WO 0046215; WO 0048603; WO 0110847; WO 9915506; WO 9734865; WO 9915507; U.S. Pat. Nos. 5,639,754; 5,952,341; WO 9825892; WO 0153262; and WO 0003873; each of which is incorporated herein by reference in its entirety.

In some embodiments, the antagonist is an amine or a phosphate salt. In some embodiments, the antagonist is a tris(hydroxymethyl)aminomethane (TRIS) salt, such as, but not limited to, 3-(2-methyl-pyrimidin-5-yl)-9-(5,6,7,8-tetrahydro-[1,8]-naphthyridin-2-yl)-nonanoic acid, 3-(pyrimidin-5-yl)-9-(5,6,7,8-tetrahydro-[1,8]-naphthyridin-2-yl)-nonanoic acid, or 3-{2-oxo-3-[3-(5,6,7,8-tetrahydro[1,8-naphthyridin-2-yl)-propyl]imidazolidin-1-yl}-3-(6-methoxy-pyridin-3-yl)-propionic acid. Salt antagonists of αᵥβ₃ are also described, for example, in U.S. Patent Application Publication No. 2002/0065291; U.S. Patent Application Publication No. 2003/0004171; U.S. Patent Application No. 2004/0249158; U.S. Patent Application Publication No. 2004/0254211; U.S. Patent Application Publication No. 2005/0101593; U.S. Patent Application Publication No. 2004/0038963; and U.S. Patent Application Publication No. 2004/0019037; each of which is incorporated herein by reference in its entirety.

In some embodiments, the antagonist is a small molecule, such as, but not limited to, (3S)-3-(3-bromo-5-chloro-2-hydroxyphenyl)-3-{[N-({5-[(5-hydroxy-1,4,5,6-tetrahydro-pyrimidin-2-yl)amino]pyridin-3-yl}carbonyl)glycyl]-amino}-propanoic acid (S247), (S)-3-(6-methoxypyridin-3-yl)-3-(2-oxo-3-(3-(5,6,7,8-tetrahydro-1,8-naphthyridin-2-yl)propyl)-imidazol-idin-1-yl)propanoic acid (L000845704 (also known as MK-0429)), or (S)-2-(8-(2-(6-(methylamino)pyridin-2-yl)ethoxy)-3-oxo-2-(2,2,2-trifluoroethyl)-2,3,4,5-tetrahydro-1H-benzo-[c]azepin-4-yl)acetic acid (SB273005). In some embodiments, the antagonist is (S)-3-(6-methoxypyridin-3-yl)-3-(2-oxo-3-(3-(5,6,7,8-tetrahydro-1,8-naphthyridin-2-yl)propyl)-imidazo-lidin-1-yl)propanoic acid (L000845704) or (S)-2-(8-(2-(6-(methylamino)pyridin-2-yl)ethoxy)-3-oxo-2-(2,2,2-trifluoroethyl)-2,3,4,5-tetrahydro-1H-benzo[c]azepin-4-yl)acetic acid (SB273005).

In some embodiments, the antagonist can be provided as a pharmaceutically acceptable salt. Such salts include, but are not limited to, pharmaceutically acceptable acid addition salts, pharmaceutically acceptable base addition salts, pharmaceutically acceptable metal salts, ammonium and alkylated ammonium salts, and combinations thereof.

Acid addition salts include salts of inorganic acids as well as organic acids. Representative examples of suitable inorganic acids include hydrochloric, hydrobromic, hydroiodic, phosphoric, sulfuric, nitric acids and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids, sulphates, nitrates, phosphates, perchlorates, borates, acetates, benzoates, hydroxynaphthoates, glycerophosphates, ketoglutarates and the like.

Base addition salts include but are not limited to, ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris (hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, e. g., lysine and arginine dicyclohexylamine and the like.

Examples of metal salts include lithium, sodium, potassium, magnesium salts and the like. Examples of ammonium and alkylated ammonium salts include ammonium, methylammonium, dimethylammonium, trimethylammonium, ethylammonium, hydroxyethylammonium, diethylammonium, butylammonium, tetramethylammonium salts and the like.

In some embodiments, the presently disclosed compounds can further be provided as a solvate.

The antagonists or their formulations can be used on a sample either in vitro (for example, on isolated cells or tissues) or in vivo in a subject (i.e. living organism, such as a patient). In some embodiments, the subject or patient is a human subject, although it is to be understood that the principles of the presently disclosed subject matter indicate that the presently disclosed subject matter is effective with respect to all vertebrate species, including mammals, which are intended to be included in the terms “subject” and “patient”. Moreover, a mammal is understood to include any mammalian species for which employing the compositions and methods disclosed herein is desirable, particularly agricultural and domestic mammalian species.

As such, the methods of the presently disclosed subject matter are particularly useful in warm-blooded vertebrates. Thus, the presently disclosed subject matter concerns mammals and birds. More particularly provided are methods and compositions for mammals such as humans, as well as those mammals of importance due to being endangered (such as Siberian tigers), of economic importance (animals raised on farms for consumption by humans), and/or of social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), and horses. Also provided is the treatment of birds, including the treatment of those kinds of birds that are endangered, kept in zoos or as pets (e.g., parrots), as well as fowl, and more particularly domesticated fowl, for example, poultry, such as turkeys, chickens, ducks, geese, guinea fowl, and the like, as they are also of economic importance to humans. Thus, also provided is the treatment of livestock including, but not limited to domesticated swine (pigs and hogs), ruminants, horses, poultry, and the like.

In some embodiments, the antagonist can include more than one of the antagonists described herein. In some embodiments, the antagonist can be administered along with one or more additional therapeutic agents known in the art for treating a disease or disorder associated with pruritis. For example, the antagonist can be co-administered with a therapeutic agent for treating AD, psoriasis, eczema, multiple sclerosis, diabetes, burn, insect bites, allergic reaction, dry skin, scars, or shingles, or a symptom thereof, e.g., pain or inflammation. The antagonist can the one or more other therapeutic agents can be provided in a single formulation or co-administered in separate formulations at about the same time or at different times (e.g., different times within the same day, week, or month).

### IV. Pharmaceutical Compositions

In some embodiments, the presently disclosed subject matter, the antagonist (which can also be referred to as the “active ingredient”) can be administered in a pharmaceutically acceptable composition where the antagonist can be admixed with one or more pharmaceutically acceptable carriers. The term “pharmaceutically acceptable carrier” means a non-toxic material that does not interfere with the effectiveness of the biological activity of the active ingredients. In some embodiments, the pharmaceutically acceptable composition can also contain salts, buffering agents, preservatives, compatible carriers, and optionally other therapeutic agents.

Suitable methods for administration of an antagonist or pharmaceutically acceptable composition thereof to a subject include, but are not limited to intravenous injection, oral administration, buccal, topical, subcutaneous administration, intraperitoneal injection, pulmonary, intanasal, intracranial injection, and rectal administration. The particular mode of administering a composition matter depends on various factors, including the distribution and abundance of cells to be treated and mechanisms for metabolism or removal of the composition from its site of administration.

An effective dose of a composition of the presently disclosed subject matter is administered to a subject. An “effective amount” is an amount of the composition sufficient to produce detectable treatment. Actual dosage levels of constituents of the compositions of the presently disclosed subject matter can be varied so as to administer an amount of the composition that is effective to achieve the desired effect for a particular subject and/or target. The selected dosage level can depend upon the activity of the composition and the route of administration.

After review of the disclosure herein of the presently disclosed subject matter, one of ordinary skill in the art can tailor the dosages to an individual subject, taking into account the particular formulation, method of administration to be used with the composition, and nature of the target to be treated. Such adjustments or variations, as well as evaluation of when and how to make such adjustments or variations, are well known to those of ordinary skill in the art.

The therapeutically effective amount can be determined by testing the compounds in an in vitro or in vivo model and then extrapolating therefrom for dosages in subjects of interest, e.g., humans. The therapeutically effective amount should be enough to exert a therapeutically useful effect in the absence of undesirable side effects in the subject to be treated with the composition.

Pharmaceutically acceptable carriers are well known to those skilled in the art and include, but are not limited to, from about 0.01 to about 0.1M and preferably 0.05M phosphate buffer or 0.8% saline. Such pharmaceutically acceptable carriers can be aqueous or non-aqueous solutions, suspensions and emulsions. Examples of non-aqueous solvents suitable for use in the presently disclosed subject matter include, but are not limited to, propylene glycol, polyethylene glycol, vegetable oils such as olive oil, and injectable organic esters such as ethyl oleate. Aqueous carriers suitable for use in the presently disclosed subject matter include, but are not limited to, water, ethanol, alcoholic/aqueous solutions, glycerol, emulsions or suspensions, including saline and buffered media. Oral carriers can be elixirs, syrups, capsules, tablets and the like.

Liquid carriers suitable for use in the presently disclosed subject matter can be used in preparing solutions, suspensions, emulsions, syrups, elixirs and pressurized compounds. The active ingredient can be dissolved or suspended in a pharmaceutically acceptable liquid carrier such as water, an organic solvent, a mixture of both or pharmaceutically acceptable oils or fats. The liquid carrier can contain other suitable pharmaceutical additives such as solubilizers, emulsifiers, buffers, preservatives, sweeteners, flavoring agents, suspending agents, thickening agents, colors, viscosity regulators, stabilizers or osmo-regulators.

Liquid carriers suitable for use in the presently disclosed subject matter include, but are not limited to, water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the carrier can also include an oily ester such as ethyl oleate and isopropyl myristate. Sterile liquid carriers are useful in sterile liquid form comprising compounds for parenteral administration. The liquid carrier for pressurized compounds disclosed herein can be halogenated hydrocarbon or other pharmaceutically acceptable propellent.

Solid carriers suitable for use in the presently disclosed subject matter include, but are not limited to, inert substances such as lactose, starch, glucose, methyl-cellulose, magnesium stearate, dicalcium phosphate, mannitol and the like. A solid carrier can further include one or more substances acting as flavoring agents, lubricants, solubilizers, suspending agents, fillers, glidants, compression aids, binders or tablet-disintegrating agents; it can also be an encapsulating material. In powders, the carrier can be a finely divided solid which is in admixture with the finely divided active compound. In tablets, the active compound is mixed with a carrier having the necessary compression properties in suitable proportions and compacted in the shape and size desired. The powders and tablets preferably contain up to 99% of the active compound. Suitable solid carriers include, for example, calcium phosphate, magnesium stearate, talc, sugars, lactose, dextrin, starch, gelatin, cellulose, polyvinylpyrrolidine, low melting waxes and ion exchange resins.

Parenteral carriers suitable for use in the presently disclosed subject matter include, but are not limited to, sodium chloride solution, Ringer's dextrose, dextrose and sodium chloride, lactated Ringer's and fixed oils. Intravenous carriers include fluid and nutrient replenishers, electrolyte replenishers such as those based on Ringer's dextrose and the like. Preservatives and other additives can also be present, such as, for example, antimicrobials, antioxidants, chelating agents, inert gases and the like.

Carriers suitable for use in the presently disclosed subject matter can be mixed as needed with disintegrants, diluents, granulating agents, lubricants, binders and the like using conventional techniques known in the art. The carriers can also be sterilized using methods that do not deleteriously react with the compounds, as is generally known in the art. The antagonists disclosed herein can take such forms as suspensions, solutions or emulsions in oily or aqueous vehicles, and can contain formulatory agents such as suspending, stabilizing and/or dispersing agents. The antagonists disclosed herein can also be formulated as a preparation for implantation or injection. Thus, for example, the antagonists can be formulated with suitable polymeric or hydrophobic materials (e.g., as an emulsion in an acceptable oil) or ion exchange resins, or as sparingly soluble derivatives (e.g., as a sparingly soluble salt). Alternatively, the active ingredient can be in powder form for constitution with a suitable vehicle, e.g., sterile pyrogen-free water, before use. Suitable formulations for each of these methods of administration can be found, for example, in Remington: The Science and Practice of Pharmacy, A. Gennaro, ed., 20th edition, Lippincott, Williams & Wilkins, Philadelphia, Pa.

For example, formulations for parenteral administration can contain as common excipients sterile water or saline, polyalkylene glycols such as polyethylene glycol, oils of vegetable origin, hydrogenated naphthalenes and the like. In particular, biocompatible, biodegradable lactide polymer, lactide/glycolide copolymer, or polyoxyethylene-polyoxypropylene copolymers can be useful excipients to control the release of active compounds. Other potentially useful parenteral delivery systems include ethylene-vinyl acetate copolymer particles, osmotic pumps, implantable infusion systems, and liposomes. Formulations for inhalation administration contain as excipients, for example, lactose, or can be aqueous solutions containing, for example, polyoxyethylene-9-auryl ether, glycocholate and deoxycholate, or oily solutions for administration in the form of nasal drops, or as a gel to be applied intranasally. Formulations for parenteral administration can also include glycocholate for buccal administration, methoxysalicylate for rectal administration, or citric acid for vaginal administration.

Further, formulations for intravenous administration can comprise solutions in sterile isotonic aqueous buffer. Where necessary, the formulations can also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachet indicating the quantity of active agent. Where the compound is to be administered by infusion, it can be dispensed in a formulation with an infusion bottle containing sterile pharmaceutical grade water, saline or dextrose/water. Where the compound is administered by injection, an ampule of sterile water for injection or saline can be provided so that the ingredients can be mixed prior to administration.

Suitable formulations further include aqueous and non-aqueous sterile injection solutions that can contain antioxidants, buffers, bacteriostats, bactericidal antibiotics and solutes that render the formulation isotonic with the bodily fluids of the intended recipient; and aqueous and non-aqueous sterile suspensions, which can include suspending agents and thickening agents.

The antagonists can further be formulated for topical administration. Suitable topical formulations include one or more compounds in the form of a liquid, lotion, cream or gel. Topical administration can be accomplished by application directly on the treatment area. For example, such application can be accomplished by rubbing the formulation (such as a lotion or gel) onto the skin of the treatment area, or by spray application of a liquid formulation onto the treatment area.

In some formulations, bioimplant materials can be coated with the compounds so as to improve interaction between cells and the implant.

Formulations of the antagonists can contain minor amounts of wetting or emulsifying agents, or pH buffering agents. The formulations comprising the compound can be a liquid solution, suspension, emulsion, tablet, pill, capsule, sustained release formulation, or powder.

The antagonists can be formulated as a suppository, with traditional binders and carriers such as triglycerides.

Oral formulations can include standard carriers such as pharmaceutical grades of mannitol, lactose, starch, magnesium stearate, polyvinyl pyrollidone, sodium saccharine, cellulose, magnesium carbonate, etc.

In some embodiments, the pharmaceutical composition comprising the antagonist of the presently disclosed subject matter can include an agent which controls release of the compound, thereby providing a timed or sustained release compound.

Peptide Modification and Preparation

It will be appreciated, of course, that the proteins or peptides of the presently disclosed subject matter may incorporate amino acid residues which are modified without affecting activity. For example, the termini may be derivatized to include blocking groups, i.e. chemical substituents suitable to protect and/or stabilize the N- and C-termini from “undesirable degradation”, a term meant to encompass any type of enzymatic, chemical or biochemical breakdown of the compound at its termini which is likely to affect the function of the compound, i.e. sequential degradation of the compound at a terminal end thereof.

Blocking groups include protecting groups conventionally used in the art of peptide chemistry which will not adversely affect the in vivo activities of the peptide. For example, suitable N-terminal blocking groups can be introduced by alkylation or acylation of the N-terminus. Examples of suitable N-terminal blocking groups include C1-C5 branched or unbranched alkyl groups, acyl groups such as formyl and acetyl groups, as well as substituted forms thereof, such as the acetamidomethyl (Acm) group. Desamino analogs of amino acids are also useful N-terminal blocking groups and can either be coupled to the N-terminus of the peptide or used in place of the N-terminal reside. Suitable C-terminal blocking groups, in which the carboxyl group of the C-terminus is either incorporated or not, include esters, ketones or amides. Ester or ketone-forming alkyl groups, particularly lower alkyl groups such as methyl, ethyl and propyl, and amide-forming amino groups such as primary amines (−NH₂), and mono- and di-alkylamino groups such as methylamino, ethylamino, dimethylamino, diethylamino, methylethylamino and the like are examples of C-terminal blocking groups. Descarboxylated amino acid analogues such as agmatine are also useful C-terminal blocking groups and can be either coupled to the peptide's C-terminal residue or used in place of it. Further, it will be appreciated that the free amino and carboxyl groups at the termini can be removed altogether from the peptide to yield desamino and descarboxylated forms thereof without affect on peptide activity.

Modifications (which do not normally alter primary sequence) include in vivo, or in vitro chemical derivatization of polypeptides, e.g., acetylation, or carboxylation. Also included are modifications of glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also embraced are sequences which have phosphorylated amino acid residues, e.g., phosphotyrosine, phosphoserine, or phosphothreonine.

Also included are polypeptides which have been modified using ordinary molecular biological techniques so as to improve their resistance to proteolytic degradation or to optimize solubility properties or to render them more suitable as a therapeutic agent. Analogs of such polypeptides include those containing residues other than naturally occurring L-amino acids, e.g., D-amino acids or non-naturally occurring or non-standard synthetic amino acids. The peptides of the presently disclosed subject matter are not limited to products of any of the specific exemplary processes listed herein.

As discussed, modifications or optimizations of peptide ligands of the presently disclosed subject matter are within the scope of the application. Modified or optimized peptides are included within the definition of peptide binding ligand. Specifically, a peptide sequence identified can be modified to optimize its potency, pharmacokinetic behavior, stability and/or other biological, physical and chemical properties.

Amino Acid Substitutions

In certain embodiments, the disclosed methods and compositions may involve preparing peptides with one or more substituted amino acid residues.

In various embodiments, the structural, physical and/or therapeutic characteristics of peptide sequences may be optimized by replacing one or more amino acid residues.

Other modifications can also be incorporated without adversely affecting the activity and these include, but are not limited to, substitution of one or more of the amino acids in the natural L-isomeric form with amino acids in the D-isomeric form. Thus, the peptide can include one or more D-amino acid resides, or can comprise amino acids which are all in the D-form. Retro-inverso forms of peptides in accordance with the presently disclosed subject matter are also contemplated, for example, inverted peptides in which all amino acids are substituted with D-amino acid forms.

The skilled artisan will be aware that, in general, amino acid substitutions in a peptide typically involve the replacement of an amino acid with another amino acid of relatively similar properties (i.e., conservative amino acid substitutions). The properties of the various amino acids and effect of amino acid substitution on protein structure and function have been the subject of extensive study and knowledge in the art.

For example, one can make the following isosteric and/or conservative amino acid changes in the parent polypeptide sequence with the expectation that the resulting polypeptides would have a similar or improved profile of the properties described above:

Substitution of alkyl-substituted hydrophobic amino acids: including alanine, leucine, isoleucine, valine, norleucine, S-2-aminobutyric acid, S-cyclohexylalanine or other simple alpha-amino acids substituted by an aliphatic side chain from C1-10 carbons including branched, cyclic and straight chain alkyl, alkenyl or alkynyl substitutions.

Substitution of aromatic-substituted hydrophobic amino acids: including phenylalanine, tryptophan, tyrosine, biphenylalanine, 1-naphthylalanine, 2-naphthylalanine, 2-benzothienylalanine, 3-benzothienylalanine, histidine, amino, alkylamino, dialkylamino, aza, halogenated (fluoro, chloro, bromo, or iodo) or alkoxy-substituted forms of the previous listed aromatic amino acids, illustrative examples of which are: 2-,3- or 4-aminophenylalanine, 2-,3- or 4-chlorophenylalanine, 2-,3- or 4-methylphenylalanine, 2-,3- or 4-methoxyphenylalanine, 5-amino-, 5-chloro-, 5-methyl- or 5-methoxytryptophan, 2′-, 3′-, or 4′-amino-, 2′-, 3′-, or 4′-chloro-, 2,3, or 4-biphenylalanine, 2′,-3′,- or 4′-methyl-2, 3 or 4-biphenylalanine, and 2- or 3-pyridylalanine.

Substitution of amino acids containing basic functions: including arginine, lysine, histidine, ornithine, 2,3-diaminopropionic acid, homoarginine, alkyl, alkenyl, or aryl-substituted (from C1-C10 branched, linear, or cyclic) derivatives of the previous amino acids, whether the sub stituent is on the heteroatoms (such as the alpha nitrogen, or the distal nitrogen or nitrogens, or on the alpha carbon, in the pro-R position for example. Compounds that serve as illustrative examples include: N-epsilon-isopropyl-lysine, 3-(4-tetrahydropyridyl)-glycine, 3-(4-tetrahydropyridyl)-alanine, N,N-gamma, gamma′-diethyl-homoarginine. Included also are compounds such as alpha methyl arginine, alpha methyl 2,3-diaminopropionic acid, alpha methyl histidine, alpha methyl ornithine where alkyl group occupies the pro-R position of the alpha carbon. Also included are the amides formed from alkyl, aromatic, heteroaromatic (where the heteroaromatic group has one or more nitrogens, oxygens, or sulfur atoms singly or in combination) carboxylic acids or any of the many well-known activated derivatives such as acid chlorides, active esters, active azolides and related derivatives) and lysine, ornithine, or 2,3-diaminopropionic acid.

Substitution of acidic amino acids: including aspartic acid, glutamic acid, homoglutamic acid, tyrosine, alkyl, aryl, arylalkyl, and heteroaryl sulfonamides of 2,4-diaminopriopionic acid, ornithine or lysine and tetrazole-substituted alkyl amino acids.

Substitution of side chain amide residues: including asparagine, glutamine, and alkyl or aromatic substituted derivatives of asparagine or glutamine.

Substitution of hydroxyl containing amino acids: including serine, threonine, homoserine, 2,3-diaminopropionic acid, and alkyl or aromatic substituted derivatives of serine or threonine. It is also understood that the amino acids within each of the categories listed above can be substituted for another of the same group.

For example, the hydropathic index of amino acids may be considered (Kyte & Doolittle, 1982, J. Mol. Biol., 157:105-132). The relative hydropathic character of the amino acid contributes to the secondary structure of the resultant protein, which in turn defines the interaction of the protein with other molecules. Each amino acid has been assigned a hydropathic index on the basis of its hydrophobicity and charge characteristics (Kyte & Doolittle, 1982), these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). In making conservative substitutions, the use of amino acids whose hydropathic indices are within +/−2 is preferred, within +/−1 are more preferred, and within +/−0.5 are even more preferred.

Amino acid substitution may also take into account the hydrophilicity of the amino acid residue (e.g., U.S. Pat. No. 4,554,101). Hydrophilicity values have been assigned to amino acid residues: arginine (+3.0); lysine (+3.0); aspartate (+3.0); glutamate (+3.0); serine (+0.3); asparagine (+0.2); glutamine (+0.2); glycine (0); threonine (−0.4); proline (−0.5.+−0.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). Replacement of amino acids with others of similar hydrophilicity is preferred.

Other considerations include the size of the amino acid side chain. For example, it would generally not be preferred to replace an amino acid with a compact side chain, such as glycine or serine, with an amino acid with a bulky side chain, e.g., tryptophan or tyrosine. The effect of various amino acid residues on protein secondary structure is also a consideration. Through empirical study, the effect of different amino acid residues on the tendency of protein domains to adopt an alpha-helical, beta-sheet or reverse turn secondary structure has been determined and is known in the art (see e.g., Chou & Fasman, 1974, Biochemistry, 13:222-245; 1978, Ann. Rev. Biochem., 47: 251-276; 1979, Biophys. J., 26:367-384).

Based on such considerations and extensive empirical study, tables of conservative amino acid substitutions have been constructed and are known in the art. For example: arginine and lysine; glutamate and aspartate; serine and threonine; glutamine and asparagine; and valine, leucine and isoleucine. Alternatively: Ala (A) leu, ile, val; Arg (R) gln, asn, lys; Asn (N) his, asp, lys, arg, gln; Asp (D) asn, glu; Cys (C) ala, ser; Gln (Q) glu, asn; Glu (E) gln, asp; Gly (G) ala; His (H) asn, gln, lys, arg; Ile (I) val, met, ala, phe, leu; Leu (L) val, met, ala, phe, ile; Lys (K) gln, asn, arg; Met (M) phe, ile, leu; Phe (F) leu, val, ile, ala, tyr; Pro (P) ala; Ser (S), thr; Thr (T) ser; Trp (W) phe, tyr; Tyr (Y) trp, phe, thr, ser; Val (V) ile, leu, met, phe, ala.

Other considerations for amino acid substitutions include whether or not the residue is located in the interior of a protein or is solvent exposed. For interior residues, conservative substitutions would include: Asp and Asn; Ser and Thr; Ser and Ala; Thr and Ala; Ala and Gly; Ile and Val; Val and Leu; Leu and Ile; Leu and Met; Phe and Tyr; Tyr and Trp. (See e.g., PROWL Rockefeller University website). For solvent exposed residues, conservative substitutions would include: Asp and Asn; Asp and Glu; Glu and Gln; Glu and Ala; Gly and Asn; Ala and Pro; Ala and Gly; Ala and Ser; Ala and Lys; Ser and Thr; Lys and Arg; Val and Leu; Leu and Ile; Ile and Val; Phe and Tyr. Various matrices have been constructed to assist in selection of amino acid substitutions, such as the PAM250 scoring matrix, Dayhoff matrix, Grantham matrix, McLachlan matrix, Doolittle matrix, Henikoff matrix, Miyata matrix, Fitch matrix, Jones matrix, Rao matrix, Levin matrix and Risler matrix (Idem.)

In determining amino acid substitutions, one may also consider the existence of intermolecular or intramolecular bonds, such as formation of ionic bonds (salt bridges) between positively charged residues (e.g., His, Arg, Lys) and negatively charged residues (e.g., Asp, Glu) or disulfide bonds between nearby cysteine residues.

Methods of substituting any amino acid for any other amino acid in an encoded peptide sequence are well known and a matter of routine experimentation for the skilled artisan, for example by the technique of site-directed mutagenesis or by synthesis and assembly of oligonucleotides encoding an amino acid substitution and splicing into an expression vector construct.

Antibody Formats and Preparation Thereof

Antibodies directed against proteins, polypeptides, or peptide fragments thereof of the presently disclosed subject matter may be generated using methods that are well known in the art. For instance, U.S. Pat. No. 5,436,157, which is incorporated by reference herein in its entirety, discloses methods of raising antibodies to peptides. For the production of antibodies, various host animals, including but not limited to rabbits, mice, and rats, can be immunized by injection with a polypeptide or peptide fragment thereof. To increase the immunological response, various adjuvants may be used depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum.

In some embodiments, one or more antibodies or fragments thereof are used. In some embodiments, one or both antibodies are single chain, monoclonal, bi-specific, synthetic, polyclonal, chimeric, human, or humanized, or active fragments or homologs thereof. In some embodiments, the antibody binding fragment is scFV, F(ab′)₂, F(ab)₂, Fab′, or Fab.

For the preparation of monoclonal antibodies, any technique which provides for the production of antibody molecules by continuous cell lines in culture may be utilized. For example, the hybridoma technique originally developed by Kohler & Milstein (1975) Nature 256:495-497, the trioma technique, the human B-cell hybridoma technique (Kozbor & Roder, 1983, Immunology Today 4:72), and the EBV-hybridoma technique (Cole et al., 1985 in Monoclonal Antibodies and Cancer Therapy, Alan R. Liss, Inc., New York, New York, United States of America, pp. 77-96) may be employed to produce human monoclonal antibodies. In some embodiments, monoclonal antibodies are produced in germ-free animals.

In accordance with the presently disclosed subject matter, human antibodies may be used and obtained by utilizing human hybridomas (Cote et al., 1983 Proc Natl Acad Sci USA 80:2026-2030) or by transforming human B cells with EBV virus in vitro (Cole et al., 1985 in Monoclonal Antibodies and Cancer Therapy, Alan R. Liss, Inc., New York, New York, United States of America, pp. 77-96). Furthermore, techniques developed for the production of “chimeric antibodies” (Morrison et al., 1984, Proc Natl Acad Sci USA. 81:6851-6855; Neuberger et al., 1984, Nature 312:604-608; Takeda et al., 1985, Nature 314:452-454) by splicing the genes from a mouse antibody molecule specific for epitopes of SLLP polypeptides together with genes from a human antibody molecule of appropriate biological activity can be employed; such antibodies are within the scope of the presently disclosed subject matter. Once specific monoclonal antibodies have been developed, the preparation of mutants and variants thereof by conventional techniques is also available.

Various techniques have been developed for the production of antibody fragments of humanized antibodies. Traditionally, these fragments were derived via proteolytic digestion of full-length antibodies (see e.g., Morimoto & Inouye, 1992, J Biochem Biophys Methods 24:107-117; Brennan et al., 1985, Science 229:81-83). However, these fragments can now be produced directly by recombinant host cells. Alternatively, Fab′-SH fragments can be directly recovered from E. coli and chemically coupled to form F(ab′)₂ fragments (Carter et al., 1992a, Proc Natl Acad Sci USA 89:4285). According to another approach, F(ab′)₂ fragments can be isolated directly from recombinant host cell culture. Other techniques for the production of antibody fragments will be apparent to the skilled practitioner. In other embodiments, the antibody of choice is a single-chain Fv fragment (scFv). See PCT International Patent Application Publication No. WO 1993/16185; U.S. Pat. Nos. 5,571,894; 5,587,458. The antibody fragment may also be a “linear antibody”, e.g., as described in U.S. Pat. No. 5,641,870, for example. Such linear antibody fragments may be monospecific or bispecific.

Humanized (chimeric) antibodies are immunoglobulin molecules comprising a human and non-human portion. More specifically, the antigen combining region (or variable region) of a humanized chimeric antibody is derived from a non-human source (e.g., murine) and the constant region of the chimeric antibody (which confers biological effector function to the immunoglobulin) is derived from a human source. The humanized chimeric antibody should have the antigen binding specificity of the non-human antibody molecule and the effector function conferred by the human antibody molecule. A large number of methods of generating chimeric antibodies are well known to those of skill in the art (see e.g., U.S. Pat. Nos. 4,975,369; 5,075,431; 5,081,235; 5,169,939; 5,202,238; 5,204,244; 5,231,026; 5,292,867; 5,354,847; 5,472,693; 5,482,856; 5,491,088; 5,500,362; and 5,502,167). Detailed methods for preparation of chimeric (humanized) antibodies can be found in U.S. Pat. No. 5,482,856. A “humanized” antibody is a human/non-human chimeric antibody that contains a minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit, or non-human primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, a humanized antibody will comprise substantially all of at least one, and typically two, variable domains, in which all or substantially all of the hypervariable loops correspond to those of a non-human immunoglobulin and all or substantially all of the FR residues are those of a human immunoglobulin sequence. The humanized antibody can optionally also comprise at least a portion of an immunoglobulin constant region (Fc), typically that of a human immunoglobulin. For further details, see e.g., Jones et al., 1986, Nature 321:522-525; Riechmann et al., 1988, Nature 332:323-327; Presta, 1992, Curr Op Struct Biol 2:593-596, PCT International Patent Application Publication No. WO 92/02190, U.S. Patent Application Publication No. 2006/0073137, and U.S. Pat. Nos. 5,225,539; 5,530,101; 5,585,089; 5,693,761; 5,693,762; 5,714,350; 5,766,886; 5,770,196; 5,777,085; 5,821,123; 5,821,337; 5,869,619; 5,877,293; 5,886,152; 5,895,205; 5,929,212; 6,054,297; 6,180,370; 6,407,213; 6,548,640; 6,632,927; 6,639,055; and 6,750,325.

In some embodiments, this presently disclosed subject matter provides for fully human antibodies. Human antibodies consist entirely of characteristically human polypeptide sequences. The human antibodies of this presently disclosed subject matter can be produced in using a wide variety of methods (see e.g., U.S. Pat. No. 5,001,065, for review).

Typically, a humanized antibody has one or more amino acid residues introduced into it from a source that is non-human. These non-human amino acid residues are often referred to as “import” residues, which are typically taken from an “import” variable domain. Humanization can be essentially performed following the method of Winter and co-workers (Jones et al, 1986, Nature 321:522-525; Riechmann et al., 1988, Nature 332:323-327); Verhoeyen et al., 1988, Science 239:1534-1536), by substituting hypervariable region sequences for the corresponding sequences of a human “acceptor” antibody. Accordingly, such “humanized” antibodies are chimeric antibodies (see e.g., U.S. Pat. Nos. 4,816,567 and 5,482,856) wherein substantially less than an intact human variable domain has been substituted by the corresponding sequence from a non-human species. In practice, humanized antibodies are typically human antibodies in which some hypervariable region residues and possibly some FR residues are substituted by residues from analogous sites in rodent antibodies.

Another method for making humanized antibodies is described in U.S. Patent Application Publication No. 2003/0017534, wherein humanized antibodies and antibody preparations are produced from transgenic non-human animals. The non-human animals are genetically engineered to contain one or more humanized immunoglobulin loci that are capable of undergoing gene rearrangement and gene conversion in the transgenic non-human animals to produce diversified humanized immunoglobulins.

In some embodiments, the choice of human variable domains, both light and heavy, to be used in making the humanized antibodies is very important to reduce antigenicity. According to the so-called “best-fit” method, the sequence of the variable domain of a rodent antibody is screened against a library of known human variable-domain sequences or a library of human germline sequences. The human sequence that is closest to that of the rodent can then be accepted as the human framework region for the humanized antibody (Sims et al., 1993, J Immunol 151:2296-2308; Chothia & Lesk, 1987, J Mol Biol 196:901-917). Another method uses a particular framework region derived from the consensus sequence of all human antibodies of a particular subgroup of light or heavy chains. The same framework may be used for several different humanized antibodies (Carter et al., 1992b, Proc Natl Acad Sci USA 89:4285; Presta et al., 1993, J Immunol 1993 151:2623). Other methods designed to reduce the immunogenicity of the antibody molecule in a human patient include veneered antibodies (see e.g., U.S. Pat. No. 6,797,492 and U.S. Patent Application Publication Nos. 2002/0034765 and 2004/0253645) and antibodies that have been modified by T-cell epitope analysis and removal (see e.g., U.S. Patent Application Publication No. 2003/0153043 and U.S. Pat. No. 5,712,120).

It is important that when antibodies are humanized they retain high affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available that illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

The antibody moieties of this presently disclosed subject matter can be single chain antibodies.

The hybrid antibodies and hybrid antibody fragments include complete antibody molecules having full length heavy and light chains, or any fragment thereof, such as Fab, Fab′, F(ab′)₂, Fd, scFv, antibody light chains and antibody heavy chains. Chimeric antibodies which have variable regions as described herein and constant regions from various species are also suitable. See for example, U.S. Patent Application No. 2003/0022244.

Fragments within the scope of the term “antibody” include those produced by digestion with various proteases, those produced by chemical cleavage and/or chemical dissociation and those produced recombinantly, so long as the fragment remains capable of specific binding to a target molecule. Among such fragments are Fab, Fab′, Fv, F(ab′)₂, and single chain Fv (scFv) fragments.

In some embodiments, the specific binding molecule is a single-chain variable analogue (scFv). The specific binding molecule or scFv may be linked to other specific binding molecules (for example other scFvs, Fab antibody fragments, chimeric IgG antibodies (e.g., with human frameworks)) or linked to other scFvs of the presently disclosed subject matter so as to form a multimer which is a multi-specific binding protein, for example a dimer, a trimer, or a tetramer. Bi-specific scFvs are sometimes referred to as diabodies, tri-specific such as triabodies and tetra-specific such as tetrabodies when each scFv in the dimer, trimer, or tetramer has a different specificity. Diabodies, triabodies and tetrabodies can also be monospecific, when each scFv in the dimer, trimer, or tetramer has the same specificity.

In some embodiments, techniques described for the production of single-chain antibodies (U.S. Pat. No. 4,946,778, incorporated by reference herein in its entirety) are adapted to produce protein-specific single-chain antibodies. In some embodiments, the techniques described for the construction of Fab expression libraries (Huse et al., 1989, Science 246:1275-1281) are utilized to allow rapid and easy identification of monoclonal Fab fragments possessing the desired specificity for specific antigens, proteins, derivatives, or analogs of the presently disclosed subject matter.

Antibody fragments which contain the idiotype of the antibody molecule can be generated by known techniques. For example, such fragments include but are not limited to: the F(ab′)₂ fragment which can be produced by pepsin digestion of the antibody molecule; the Fab′ fragments which can be generated by reducing the disulfide bridges of the F(ab′)₂ fragment; the Fab fragments which can be generated by treating the antibody molecule with papain and a reducing agent; and Fv fragments.

The generation of polyclonal antibodies is accomplished by inoculating the desired animal with the antigen and isolating antibodies which bind the antigen therefrom at any epitopes present therein.

Monoclonal antibodies directed against full length or peptide fragments of a protein or peptide may be prepared using any well known monoclonal antibody preparation procedures, such as those described, for example, in Harlow & Lane, 1988, Antibodies, A Laboratory Manual, Cold Spring Harbor Laboratory Publications, Cold Spring Harbor, New York, United States of America; Tuszynski et al., 1988, Blood 72:109-115). Quantities of the desired peptide may also be synthesized using chemical synthesis technology. Alternatively, DNA encoding the desired peptide may be cloned and expressed from an appropriate promoter sequence in cells suitable for the generation of large quantities of peptide. Monoclonal antibodies directed against the peptide are generated from mice immunized with the peptide using standard procedures as referenced herein.

Exemplary complementarity-determining region (CDR) residues or sequences and/or sites for amino acid substitutions in framework region (FR) of such humanized antibodies having improved properties such as, e.g., lower immunogenicity, improved antigen-binding or other functional properties, and/or improved physicochemical properties such as, e.g., better stability, are provided.

The presently disclosed subject matter encompasses more than the specific fragments and humanized fragments disclosed herein. In some embodiments, the antibody is selected from the group consisting of a single chain antibody, a monoclonal antibody, a bi-specific antibody, a chimeric antibody, a synthetic antibody, a polyclonal antibody, or a humanized antibody, or active fragments or homologs thereof.

A nucleic acid encoding the monoclonal antibody obtained using the procedures described herein may be cloned and sequenced using technology which is available in the art, and is described, for example, in Wright et al., 1992. Critical Rev in Immunol 12(3,4):125-168) and the references cited therein. Further, the antibody of the presently disclosed subject matter may be “humanized” using the technology described in Wright et al., 1992 and in the references cited therein, and in Gu et al., 1997, Thromb Haemost 77(4):755-759.

To generate a phage antibody library, a cDNA library is first obtained from mRNA which is isolated from cells, e.g., the hybridoma, which express the desired protein to be expressed on the phage surface, e.g., the desired antibody. cDNA copies of the mRNA are produced using reverse transcriptase. cDNA which specifies immunoglobulin fragments are obtained by PCR and the resulting DNA is cloned into a suitable bacteriophage vector to generate a bacteriophage DNA library comprising DNA specifying immunoglobulin genes. The procedures for making a bacteriophage library comprising heterologous DNA are well known in the art and are described, for example, in Green & Sambrook, 2012, Molecular Cloning: A Laboratory Manual, Fourth Edition, Cold Spring Harbor Laboratory Press, Cold Spring Harbor, New York, United States of America.

Bacteriophage which encode the desired antibody, may be engineered such that the protein is displayed on the surface thereof in such a manner that it is available for binding to its corresponding binding protein, e.g., the antigen against which the antibody is directed. Thus, when bacteriophage which express a specific antibody are incubated in the presence of a cell which expresses the corresponding antigen, the bacteriophage will bind to the cell. Bacteriophage which do not express the antibody will not bind to the cell. Such panning techniques are well known in the art.

Processes such as those described above, have been developed for the production of human antibodies using M13 bacteriophage display (Burton & Barbas, 1994, Adv Immunol 57:191-280). Essentially, a cDNA library is generated from mRNA obtained from a population of antibody-producing cells. The mRNA encodes rearranged immunoglobulin genes and thus, the cDNA encodes the same. Amplified cDNA is cloned into M13 expression vectors creating a library of phage which express human Fab fragments on their surface. Phage which display the antibody of interest are selected by antigen binding and are propagated in bacteria to produce soluble human Fab immunoglobulin. Thus, in contrast to conventional monoclonal antibody synthesis, this procedure immortalizes DNA encoding human immunoglobulin rather than cells which express human immunoglobulin.

The procedures just presented describe the generation of phage which encode the Fab portion of an antibody molecule. However, the presently disclosed subject matter should not be construed to be limited solely to the generation of phage encoding Fab antibodies. Rather, phage which encode single chain antibodies (scFv/phage antibody libraries) are also included in the presently disclosed subject matter. Fab molecules comprise the entire Ig light chain, that is, they comprise both the variable and constant region of the light chain, but include only the variable region and first constant region domain (CH1) of the heavy chain. Single chain antibody molecules comprise a single chain of protein comprising the Ig Fv fragment. An Ig Fv fragment includes only the variable regions of the heavy and light chains of the antibody, having no constant region contained therein. Phage libraries comprising scFv DNA may be generated following the procedures described in Marks et al., 1991, J Mol Biol 222:581-597. Panning of phage so generated for the isolation of a desired antibody is conducted in a manner similar to that described for phage libraries comprising Fab DNA.

The presently disclosed subject matter should also be construed to include synthetic phage display libraries in which the heavy and light chain variable regions may be synthesized such that they include nearly all possible specificities (Barbas, 1995, Nature Medicine 1:837-839; de Kruif et al., 1995, J Mol Biol 248:97-105).

In the production of antibodies, screening for the desired antibody can be accomplished by techniques known in the art, e.g., ELISA (enzyme-linked immunosorbent assay). Antibodies generated in accordance with the presently disclosed subject matter may include, but are not limited to, polyclonal, monoclonal, chimeric (i.e., “humanized”), and single chain (recombinant) antibodies, Fab fragments, and fragments produced by a Fab expression library.

By way of example and not limitation, E. coli can be used as a host for recombinant protein production, including immunoglobulin fragments, as can mammalian cells. E. coli can be employed to produce target proteins including but not limited to the scFvs and variants thereof of the presently disclosed subject matter in large quantities (see e.g., Verma et al., 1998, Journal of Immunological Methods 216(1-2), 165-181).

Since scFvs contain 2 disulfide bonds, a leader sequence (PelB) to direct the antibody fragment into the E. coli periplasmic space can also be used as desired. The leader can then be removed physiologically once the scFv reaches the periplasmic space. The latter space between the inner and outer membranes of Gram negative bacteria is more oxidizing compared to the cytoplasm as it contains chaperonin equivalents and disulfide isomerases (Skerra & Pluckthun, 1988, Science 240:1038).

Substantially pure peptide obtained as described herein may be purified by following known procedures for protein purification, wherein an immunological, enzymatic, or other assay is used to monitor purification at each stage in the procedure. Protein purification methods are well known in the art, and are described, for example in Deutscher et al., 1990, Guide to Protein Purification, Harcourt Brace Jovanovich, San Diego, Calif., United States of America.

In some embodiments, when used in vivo for therapy, the antibodies of the subject presently disclosed subject matter are administered to the subject in therapeutically effective amounts (i.e., amounts that have desired therapeutic effect). They will normally be administered parenterally. The dose and dosage regimen will depend upon the degree of the infection, the characteristics of the particular antibody or immunotoxin used, e.g., its therapeutic index, the patient, and the patient's history. Advantageously the antibody or immunotoxin is administered continuously over a period of 1-2 weeks. Optionally, the administration is made during the course of adjunct therapy such as antimicrobial treatment, or administration of tumor necrosis factor, interferon, or other cytoprotective or immunomodulatory agent.

In some embodiments, for parenteral administration, the antibodies will be formulated in a unit dosage injectable form (solution, suspension, emulsion) in association with a pharmaceutically acceptable parenteral vehicle. Such vehicles are inherently nontoxic, and non-therapeutic. Examples of such vehicle are water, saline, Ringer's solution, dextrose solution, and 5% human serum albumin. Nonaqueous vehicles such as fixed oils and ethyl oleate can also be used. Liposomes can be used as carriers. The vehicle can contain minor amounts of additives such as substances that enhance isotonicity and chemical stability, e.g., buffers and preservatives. The antibodies will typically be formulated in such vehicles at concentrations of about 1.0 mg/ml to about 10 mg/ml.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter.

### Example 1

### Experimental Model and Methods for Example 2

Table 2, below, lists reagents and resources used in the Examples.

TABLE 2
Reagents/Resources Used in the Examples

| | | |
|---|---|---|
| Reagent or Resource | Source | Identifier |

Antibodies and Primers

| | | |
|---|---|---|
| Anti-Calnexin | Sigma Aldrich | C4731 |
| Anti-STAT6 | R&D Systems | AF2167 |
| Anti-pSTAT6 | Sigma Aldrich | SAB 450546 |
| Anti-STAT3 | Thermofisher | MA1-13042 |
| Anti-pSTAT3 | Thermofisher | 44-384G |
| Anti-pSTAT5 | Sigma Aldrich | SAB4301474 |
| Anti-STAT5 | Thermofisher | PA5-36075 |
| Anti-GAPDH | Santa Cruz Biotechnology | sc-32233 |
| Anti-Periostin | Thermofisher | PA5-34641 |
| Anti-TSLPR | ProSci | 4209 |
| Anti-ITGB3 | Novus Biological | NBP1-60872 |
| Anti-Tuj | Abcam | ab81887 |
| Goat Anti-Rabbit Cy3 | Life Technologies | A10520 |
| Goat anti-rabbit IgG-HRP | Santa Cruz Biotechnology | sc-2004 |
| Goat anti-mouse IgG HRP | Santa Cruz Biotechnology | sc-2005 |
| Goat Anti-Mouse 488 | Life Technologies | A11001 |
| Taqman Mouse ITGAV Probe Set | Thermofisher | Mm01339538 |
| Taqman Mouse ITGB3 Probe Set | Thermofisher | Mm1240368 |
| Taqman Mouse ITGB5 Probe Set | Thermofisher | Mm00439825 |
| Taqman Mouse ITGA2b Probe Set | Thermofisher | Mm00439747 |
| Taqman Mouse GAPDH Probe Set | Thermofisher | Mm99999615 |
| Taqman Dog ITGAV Probe Set | Thermofisher | Cf02696697 |
| Taqman Dog ITGB3 Probe Set | Thermofisher | Cf02623437 |
| Taqman Dog ITGB5 Probe Set | Thermofisher | Cf02658863 |
| Taqman Dog ITGA2b Probe Set | Thermofisher | Cf02623652 |
| Taqman Dog GAPDH Probe Set | Thermofisher | Cf04419463 |

Biological Samples

| | | |
|---|---|---|
| Mast cells | Bone marrow (C57BL6J) | N/A |

Mouse Lines Utilized

| | | |
|---|---|---|
| C57Bl/6J | Jackson Labs | Strain Code: 000664 |
| C57BL/6-Itgb3tm1.1Wlbcr/J | Jackson Labs | Strain Code: 028232 |
| B6J.Cg-Ssttm2.1(cre)Zjh/MwarJ | Jackson Labs | Strain Code: 028864 |
| B6.129-Trpv1tm1(cre)Bbm/J | Jackson Labs | Strain Code: 017769 |
| B6.129X1-Trpv1tm1Jul/J | Jackson Labs | Strain Code: 003770 |
| Trpa1tm2.1Kykw/J | Jackson Labs | Strain Code: 008654 |
| NOD.CB17-Prkdc^{scid}/J | Jackson Labs | Strain Code: 001303 |
| NOD/ShiLtJ | Jackson Labs | Strain Code: 001976 |
| B6.129S7-Rag1^{tm1Mom}/J | Jackson Labs | Strain Code: 002216 |
| B6.Cg-Gt(ROSA)26Sor^{tm9(CAG-tdTomato)Hze}/J | Jackson Labs | Strain Code: 007909 |
| Kit^{W-sh}/HNihrJaeBsmJ | Jackson Labs | 005051 |
| NC/Nga | Charles River Lab (Japan) | RBRC01059 |

Chemicals, Peptides, and Recombinant Proteins

| | | |
|---|---|---|
| Recombinant Mouse Periostin | R&D Biosystems | Catalog # 2955-F2 |
| Recombinant mouse IL-3 | R&D Biosystems | Catalog # 403-ML |
| Recombinant mouse SCF | R&D Biosystems | Catalog # 455-MC |
| Mouse anti-DNP IgE (clone SPE-7) | Sigma Aldrich | D8406 |
| DNP-HAS | Sigma Aldrich | A6661 |
| Ionomycin calcium salt | Invitrogen | I24222 |
| Dispase | Fisher Scientific | NC9886504 |
| Collagenase | MP Biomedicals | 150704 |
| Laminin | Sigma Aldrich | L2020 |
| DMEM | Atlanta Biologicals | 10-013-CV |
| RPMI-1640 | Corning, NY | 10-040-CM |
| Pefabloc | Sigma Aldrich | 15633 |
| FBS | Atlanta Biologicals | S11050 |
| PenStrep | VWR | K952 |
| Poly-L-Lysine | Sigma Aldrich | P4704 |
| Fura-2 AM | Enzo | ENZ-52006 |
| Recombinant Mouse TSLP | R&D Biosystems | 555-TS |
| Histamine | Sigma Aldrich | H125 |
| Chloroquine | Sigma Aldrich | C6628 |
| Allyl isothiocyanate (AITC) | Sigma Aldrich | W203408 |
| Capsaicin | Sigma Aldrich | M2028 |
| Cilengitide | Sigma Aldrich | SMC1594 |
| Niclosamide | Tocris Biosciences | 4079 |
| SD 1008 | Tocris Biosciences | 3035 |
| MC903 | Tocris Biosciences | 2700 |
| House Dust Mites (Dermatophagoides farina) | Greer, Lenoir, NC, USA | NC1563903 |
| Betamethasone dipropionate | Sigma Aldrich | 5593 |

Commercially Available Kits Used

| | | |
|---|---|---|
| In vivo jetPEI | Polyplus | 201-10G |
| Qiagen RNEasy Mini Kit | Qiagen | 74704 |
| Mouse Periostin/OSF-2 DuoSet ELISA | R&D Biosystems | DY2955 |

Software and Algorithms

| | | |
|---|---|---|
| ImageJ | NIH | NA |
| Prism8 | Graphpad | NA |
| pClamp10 software | Axon Instruments | NA |

Equipment, Microscopes, and Behavior

| | | |
|---|---|---|
| Calcium Imaging Scope | Nikon | TE200 |
| Axopatch-700B amplifier | Axon Instrument | |
| Eclipse Ti | Nikon, Melville (NY) | |
| Dynamic Plantar Aesthesiometer | Ugo Basile, Italy | 37450 |
| Rotarod Rotomex 5 | Columbus Instruments | |
| Hargreave's Apparatus | Ugo Basile, Italy | |
| Cold Test | Dry Ice assay | (Brenner et al., 2012) |
| BioTek Neo2 multimode plate reader | BioTek | |

### Animals:

Mice were housed in small social groups (4 animals) in individually ventilated cages under 12-hour light/dark cycles and fed ad libitum. 8-12-week old animals of both genders were used in all experiments. C57BL/6N and all other genetically modified and knockout (KO) mice (Trpv1-cre; Sst-IRES-Cre; TRPV1 KO; TRPA1 KO; mast cells deficient c-kit mice; B & T cells deficient RAG KO mice and controls; B, T, and NK cells KO mice NOD/SCID and its control NOD) were purchased from the Jackson laboratory (Ellsworth, Maine, United States of America). TRPV1, TRPA1, and double KO mice were bred in house. Trpv1-IRES-Cre animals were bred to a foxed β3 allele (Morgan et al., 2010), allowing a conditional deletion of β3 in sensory neurons. Sst-IRES-Cre knock-in line was crossed to conditional alleles, to enable the Cre-dependent expression of tdTomato (Ai9) (Madisen et al., 2010) from the R26 locus. Genotyping of offspring from all breeding steps was performed with genomic DNA isolated from tail snips and allele-specific primer pairs.

Itch and Pain Behavioral Measurements:

All behavioral experiments were conducted during the light cycle at ambient temperature (23° C.). Behavioral assessment of scratching behavior was conducted as described previously (Mishra et al., 2011). Briefly, mice were injected intradermal into the nape of the neck with periostin (R&D), histamine, and chloroquine (all Millipore-Sigma) as previously described (Shimada and LaMotte, 2008). Compounds were diluted in PBS and the same was used as a vehicle. For inhibitor study, cilengitide was first injected intradermal (i.d.) to observe any unwanted effect on itch behavior. In separate experiments, cilengitide was injected intravenously (i.v.) and intraperitoneal (i.p.) 10 minutes prior to periostin injection in the dorsal neck. Additionally, cilengitide and periostin were combined together (mix) and injected i.d. into the dorsal neck of mouse. Scratching behavior was recorded for 30 minutes and data was presented in bouts per 30 minutes for mice. One bout was defined as scratching behavior toward the injection site between lifting the hind leg from the ground and either putting it back on the ground or guarding the paw with the mouth. Injections of periostin, and capsaicin in the mouse cheek itch/pain model were performed as described previously (Shimada and LaMotte, 2008). For eye wipes assay, periostin and capsaicin were dropped on mouse cornea and counted wipes for 1-minute. Injection volume was always 20 μl in mice. For dog's study, periostin (25 μg/25 μl) was injected in the dorsal neck (s.c.) and behavior was recorded and quantified for 30 minutes as “duration of pruritus manifestation” (DPM), as described earlier (Paps et al., 2016). For non-human primate (NHP) studies, periostin (25 μg/100 μl) was injected in the NHP thigh (s.c.) on the lateral side of the upper part of the hind limb; the skin area over the vastus lateralis muscle. The lateral side of the upper part of the hind limb was chosen as an injection site because this location is safe and easy to access when the animal is in a chair. The number of scratches is easily countable in NHP. When different raters separately scored a single tape, the ratings indicated high interrater reliability (coefficient of correlation, r>0.95). This method of itch readout in NHP has been used and accepted (Ko and Naughton, 2000). The behavior was recorded for 30 minutes duration and number of scratches quantified. In NHP, a scratch is defined as one brief (<1 s) episode of scraping contact of the forepaw or hind paw on the skin surface

For pain measurement, a study was performed as described previously (Mishra and Hoon, 2013; Pogorzala et al., 2013). Mice were acclimatized to plexiglass chambers for 20 minutes and Hargreaves (hot), Dry ice assay (cold), von-Frey (mechanical), and Rotarod (proprioception) were performed on genotypes. Each mouse was recorded twice, and average of each measurements were presented. Blinded assessment of mouse behavioral experiments between genotypes and treatment groups was performed.

Allergic Itch Model to Quantify Chronic Itch and Measurement of Periostin using ELISA:

C57BL6 mice (Jackson Labs, Ellsworth, Maine, United States of America) were applied daily with MC903 (4 nmol) and vehicle (97% ethanol) after brief anesthesia. Skin thickness was measured using cutimeter as described (Fukuyama et al., 2015). Scratching behavior was video-monitored for 30 min on Day 1 and Day 7. On Day 7 skin was collected from vehicle and MC903 treated mice for the Western Blot (WB) as described below. NC/Nga mice (Charles River, Yokohama, Japan) were sensitized and challenged with house dust mite (HDM) allergen (Dermatophagoides farina, Greer, Lenoir, N.C., United States of Amercia) as described previously (Fukuyama et al., 2018). In short, for sensitization, 30 μl of HDM in mineral oil (10 mg/ml) was applied topically to the clipped back twice weekly supported by tape stripping until visible lesions had developed. After development of visible lesions mice were treated daily either with vehicle cream or betamethasone dipropionate (0.1% in lipoderm, n=8). Application of betamethasone dipropionate was reduced to every other day on day 26 because of significant weight loss). Scratching behavior was video monitored for 60 min period immediately after HDM on day 42. For periostin measurement, mice were sacrificed for determination of periostin in skin on day 43. A portion of back skin tissue were snap-frozen in liquid nitrogen. Briefly, samples were homogenized under liquid nitrogen, and the homogenates were taken in 200 μL RPMI 1640 medium containing 1 mmol/L Pefabloc. The amount of periostin was determined using ELISA according to the manufacturer's instruction.

### DRG Cell Culture:

DRGs were isolated from mice and dissociated in 1 mL of media containing 2.5 U/mL of dispase (Fisher Scientific, Hampton, New Hampshire, United States of America) and 2.5 mg/mL of collagenase (Fisher Scientific, Hampton, New Hampshire, United States of America). After dissociation, the cells were washed with complete media (DMEM (HiMedia Laboratories, Mumbai, India) with 10% FBS (Atlanta Biologicals, Flowery Branch, Georgia, United States of America) and 1% PenStrep (VWR International, Radnor, Pa., United States of America)) and pelleted at 1000 rpm for 15 minutes. Approximately 30 μL of the cell suspension was plated on 18 mm round glass slides with a coating of laminin (Sigma Aldrich, St. Louis, Miss., United States of America) and poly-L-lysine (Sigma Aldrich, St. Louis, Miss., United States of America) and incubated for 1.5 hours. Afterwards, 1 mL of complete media was added, and the cells were incubated overnight. All incubation steps were done at 37° C. with 5% CO₂.

### Mast Cell Release and Degranulation:

Mouse bone marrow-derived mast cells were cultured from femurs of C57B1/6J mice as described (Jensen et al., 2006). Degranulation was assessed by measuring β-hexoseaminidase release as described (Cruse et al., 2013) using mast cells sensitized with 100 ng/mL anti-DNP IgE (SPE7 clone) (Sigma Aldrich, St. Louis, Miss., United States of America) for 16 hours, before the cells were challenged for 30 minutes with the indicated stimulus.

### Calcium Imaging on Mast Cells:

Changes in cytosolic Ca²⁺ were assayed using ratiometric Fura-2 AM measurements as described (Cruse et al., 2013). Fluorescence was measured at two excitation wavelengths (340 and 380 nm) and an emission wavelength of 510 nm using a BioTek Neo2 multimode plate reader (BioTek, Winooski, Vt., United States of America). The ratio of fluorescence readings was calculated following subtraction of background fluorescence of cells not loaded with Fura-2 AM.

### Immunohistochemistry:

DRGs were dissected from mice with various genotypes. Double and single IHC were performed as previously described (Mishra & Hoon, 2013). Images were collected on an Eclipse Ti (Nikon, Melville, N.Y., United States of America) fluorescent microscope. Sections were selected randomly, and counting was performed on each DRG section and presented as mean of 3-5 sections from each mouse.

Quantification of Periostin from Murine Keratinocyte Cell Line Culture:

The murine keratinocyte cell line (Balb/MK2) was used in this study to determine the periostin production induced by TSLP. Cells were cultured in EMEM medium according to the previously described method (Fukuyama et al., 2018). Confluent cells were exposed to TSLP at 1 or 10 ng/ml in FBS-free medium for 24 hrs. Inhibitory effect of the JAK2 inhibitor, SD 1008 and the STAT3 inhibitor, niclosamide, on TSLP-induced periostin production was also quantified using the murine Balb/MK2 keratinocyte cell line. Confluent cells were pre-exposed for 4 hrs to SD 1008 (10 μmol/l) or niclosamide (10 μmol/l), before being exposed to the TSLP at 10 ng/ml for a further 24 h. After TSLP exposure, periostin levels in cell supernatant were determined by ELISA according to the manufacturer's instructions.

Calcium Imaging:

Before imaging, cells were incubated in 350 μL of complete media containing 1 μM Fura-2 AM (Enzo Life Sciences, Farmingdale, New York, United States of America) for 30 min at 37° C. with 5% CO₂. During imaging, the cells were perfused with a buffer containing the following: 135 mM sodium chloride, 3.2 mM potassium chloride, 2.5 mM magnesium chloride, 2.8 mM calcium chloride, 667 μM monobasic sodium phosphate, 14.2 mM sodium bicarbonate, and 10.9 mM D-glucose (all from VWR International, Radnor, Pennsylvania, United States of America) with a pH between 7.00 and 7.40. The buffer and the holding plate were kept at 37° C. while imaging. Imaging data was collected on a TE200 inverted microscope using NIS Elements software (Nikon, Melville, New York, United States of America). Cells were exposed to 340 nm and 380 nm wavelengths for 100 ms and the A₃₄₀/A₃₈₀ ratio was calculated. Traces were analyzed using Excel and responses greater than 10% of the baseline were counted. Each data point in the scatter plots represented one coverslip.

### Whole-Cell Patch Clamp Recordings in Mouse DRG Neurons:

DRGs were removed aseptically from SOM-reporter mice (6-8 weeks) and incubated with collagenase (1.25 mg/ml)/dispase-II (2.4 units/ml) (both from Roche, Basel, Switzerland) at 37° C. for 90 min, then digested with 0.25% trypsin for 8 min at 37° C., followed by 0.25% trypsin inhibitor. Cells were mechanically dissociated with a flame polished Pasteur pipette in the presence of 0.05% DNAse I (Sigma, St. Louis, Miss., United States of America). DRG cells were plated on glass cover slips and grown in a neurobasal defined medium (with 2% B27 supplement, Invitrogen, Carlsbad, Calif., United States of America) with 5 μM AraC and 5% carbon dioxide at 36.5° C. DRG neurons were grown for 24 hours before use. Whole-cell patch clamp recordings were performed at room temperature using an Axopatch-700B amplifier (Axon Instruments, Foster City, Calif., United States of America) with a Digidata 1440B (Axon Instruments, Foster City, Calif., United States of America). Only SOM-positive neurons (<20 mM) were recorded. The patch pipettes were pulled from borosilicate capillaries (World Precision Instruments, Inc., Sarasota, Fla., United States of America) using a P-97 Flaming/Brown micropipette puller (Sutter Instrument Co., Novato, Calif., United States of America). Pipette resistance was 4-6 MΩ for whole-cell recording of periostin—induced inward currents, as previously recorded (Han et al., Neuron, 2018, PMID: 30033153)

### Reverse Transcription-PCR:

RNA was isolated from fresh-frozen lumbar DRG from mice, dogs and NHP using RNA easy kit (Qiagen, Germantown, Md., United States of America) according to the manufacturer protocol. To synthesize cDNA 200 ng of RNA was used with 2 μL random hexamer primers (Invitrogen, Carlsbad, Calif., United States of America) and SmartScribe Reverse Transcriptase (Clontech, Mountain View, Calif., United States of America), as described previously (Mishra and Hoon, 2013). Taqman probes for all genes were purchased from Invitrogen. All samples were run on an Applied Biosystems StepOnePlus Real Time PCR System using Taqman Gene Expression Master Mix (Cat # 4369016, Applied Biosystems, Foster City, Calif., United States of America) with the recommended qPCR cycle. CT values were calculated using StepOne Software v2.2.2 (Applied Biosystems, Foster City, Calif., United States of America). GAPDH was used as a housekeeping gene for normalization. Relative tissue expression values were calculated using the following equation: relative expression=2^{-ΔCT}.

### Western Blot (WB):

To extract total protein, dorsal root ganglia, and skin were homogenized using a tissue homogenizer in the presence of 100 μl of ice cold RIPA buffer supplemented with protease inhibitor tablets (Pierce™ Biotechnolgy, Rockford, Ill., United States of America). Total protein of lysates was measured using standard BCA (Bicinchoninic Acid Assay). Protein lysates were then denatured by heating at 95° C. in Laemmli's buffer containing 2% w/v SDS, 62.5 mM Tris (pH 6.8), 10% glycerol, 50 mM DTT, and 0.01% w/v bromophenol blue. The lysates were cooled on ice and briefly micro-centrifuged. Aliquots of 35 μg of protein were loaded onto a 10% SDS-PAGE gel, and subsequently electro blotted onto PVDF membranes. Membranes were incubated in 15 ml of blocking buffer (20 mM Tris base and 140 mM NaCl, 5% bovine serum albumin, and 0.1% Tween-20) for 1 hour. Membranes were then incubated with the desired primary antibody diluted in 10 ml of blocking buffer at 4° C. overnight. Next day membrane was washed and incubated with an appropriate horseradish peroxidase-conjugated secondary antibody (1:1000) to detect proteins in 10ml blocking buffer for 1 hour at room temperature. Immuno-reactive proteins were revealed using enhanced chemiluminescence detection (Pierce ECL, Pierce Biotechnology, Rockford, Ill., United States of America). Densitometry analysis was performed using open sourced ImageJ software from NIH. Anti-TSLP receptor antibody was used at 1 ng/ml. All other primary antibodies were used at a dilution of 1:1000. Secondary anti-rabbit and anti-mouse antibodies were purchased from Santa Cruz Biotechnology (Dallas, Tex., United States of America) and used in 1:1000 dilution.

### Statistical Analysis:

Statistical analyses and graphs were made in Prism 8 (GraphPad Software, La Jolla, Calif., United States of America). Differences between mean values were analyzed using unpaired one-tailed and two-tailed Student's t-test as appropriate or 1-way/2-way analysis of variances (ANOVA) with Dunn's multiple comparisons post hoc test when more than two data groups were compared. Differences were considered significant for *p<0.05. p values, definition, and number of replicates as well as definitions of center and dispersion were given in the respective figure legend. No statistical method was employed to predetermine sample sizes. The sample sizes used in our experiments were similar to those generally used in the field.

### Example 2

### Periostin Activtion of Integrin Receptors on Sensory Neurons and Induction of Itch

### Periostin Induces Itch in Mice, Dogs, and Monkeys:

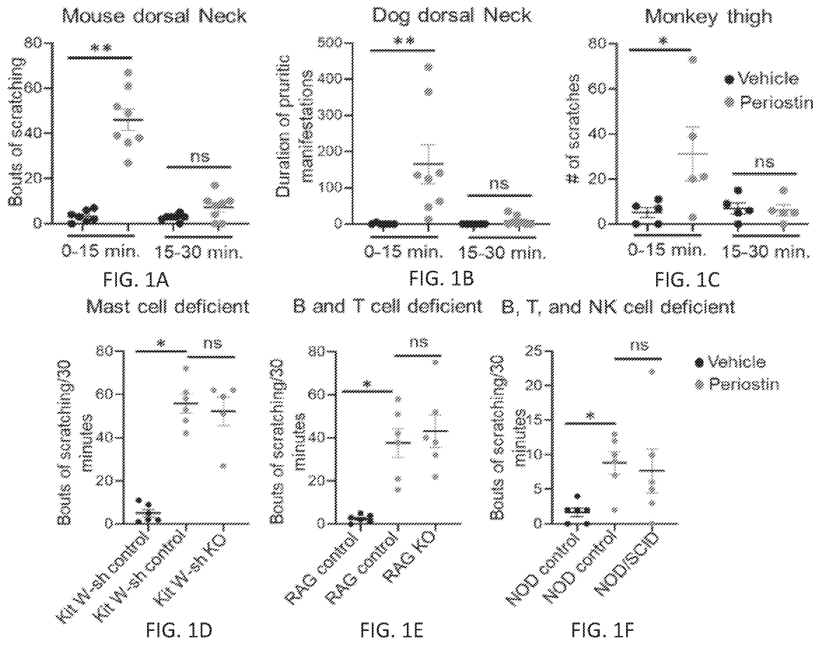

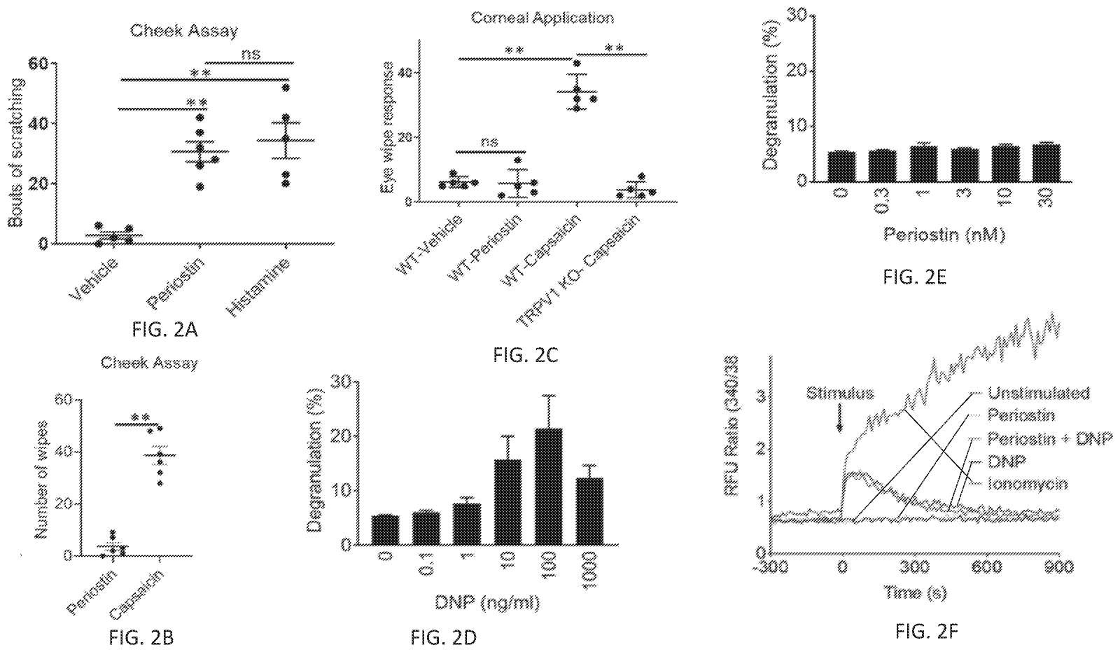

Periostin is produced in large amounts in the skin of patients suffering from pruritic dermatoses such as AD and psoriasis (Merryman-Simpson et al., 2008; Mineshige et al., 2018). To determine the possible role of periostin as a pruritogen, intradermal (i.d.) injections of periostin in mice were studied to see if they triggered itch behavior. Surprisingly, a single injection of periostin in the dorsal neck of mice induced robust scratching behavior within 15 minutes of an intracutaneous injection. See FIG. 1A. As somatosensory neurons are involved in both itch, pain and touch, a cheek injection model that is known to permit the discrimination of pain and itch behaviors in mice (Kardon et al., 2014; Shimada and LaMotte, 2008) was then studied. Interestingly, periostin injections in the cheek caused a robust scratching behavior similar to that of histamine while it did not induce wiping when compared to capsaicin, the archetypal pain inducer in mice. See FIGS. 2A and 2B. Next, it was determined if periostin induced pain behavior by directly applying it into the cornea, as only nociceptive compounds, such as capsaicin, induce a wiping behavior when added to the eye of mice (Mishra and Hoon, 2010). No eye-wiping response to the ocular application of periostin was observed, while capsaicin caused a robust eye-wipe behavior in wild-type mice compared to TRPV1 knockout mice. See FIG. 2C.

Many exogenous and endogenous molecules—for example histamine—have been shown to induce itch in mice, but, except for IL-31, most of them are not conserved pruritogens among animal species or humans (Olivry and Baumer, 2015). Recombinant mouse periostin has an approximately 85 and 90% amino acid homology with that of monkeys and dogs, respectively. To assess if periostin induced itch in these higher mammalian species, mouse recombinant periostin (25 μg/100 μl) was injected intradermally in dogs and subcutaneously in monkeys. Excitingly, periostin induced a robust scratching within 15 minutes of injection, irrespective of the route (intra- or sub-cutaneous) and body site (neck or thigh) of administration; meanwhile injections of the control had no influence on itch manifestations. See FIGS. 1B and 1C. Taken together, these results show that periostin acts as a strong pruritogen with a behavioral response that is conserved among mice, dogs, and monkeys.

As many mediators derived from several immune cell types can activate sensory neurons to induce itch, studies were conducted to determine if the periostin-induced pruritus was due to the direct (primary) or indirect (secondary) stimulation of sensory neurons. To determine if the periostin-induced itch followed mast cell activation, dinitrophenyl (DNP)-specific IgE-sensitized mast cells were challenged with increasing concentrations of the hapten DNP or periostin. While, as expected, DNP induced the degranulation of mast cells sensitized with anti-DNP IgE, while periostin did not. See FIGS. 2D and 2 E. Furthermore, no periostin-dependent change in calcium release was observed when mast cells were directly stimulated with periostin alone, and there was no enhancement of the DNP-induced calcium responses after the addition of periostin to DNP-sensitized mast cells. See FIG. 2F. Similarly, to exclude the possibility that the stimulation of skin resident or infiltrating immune cells by periostin could release mediators that would indirectly stimulate somatosensory neurons and cause itch, periostin was injected into the neck of mice deficient in mast cells (Kit W-sash) (Grimbaldeston et al., 2005), B and T cells (Rag1^{-/-}),and B, T, and NK cells (NOD/SCID) (Bosma et al., 1983; Mombaerts et al., 1992; Shultz et al., 1995). The induced itch response was then compared with that of control littermates. Interestingly, similar scratching bouts were detected in the mast cell-, B and T cell- or B, T, and NK cell-deficient and control mice. See FIGS. 1D, 1E, and 1F. Altogether, these data suggest that periostin-evoked itch does not specifically require mast cells, lymphocytes or pruritogens released when these cells are activated. In contrast, without being bound to any one theory, the results suggest that periostin can induce itch via the direct activation of somatosensory neurons.

### Integrin Receptors for Periostin are Present in DRG Somatosensory Neurons:

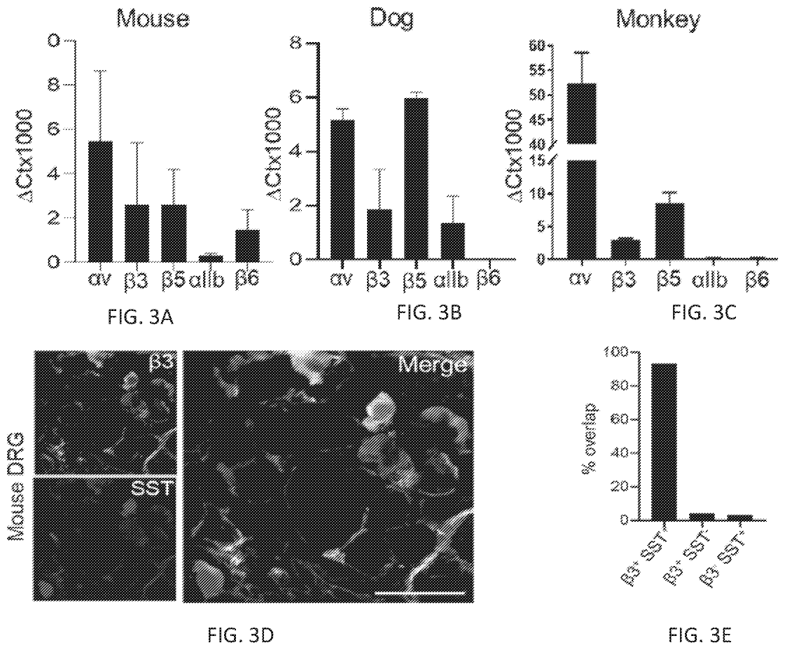

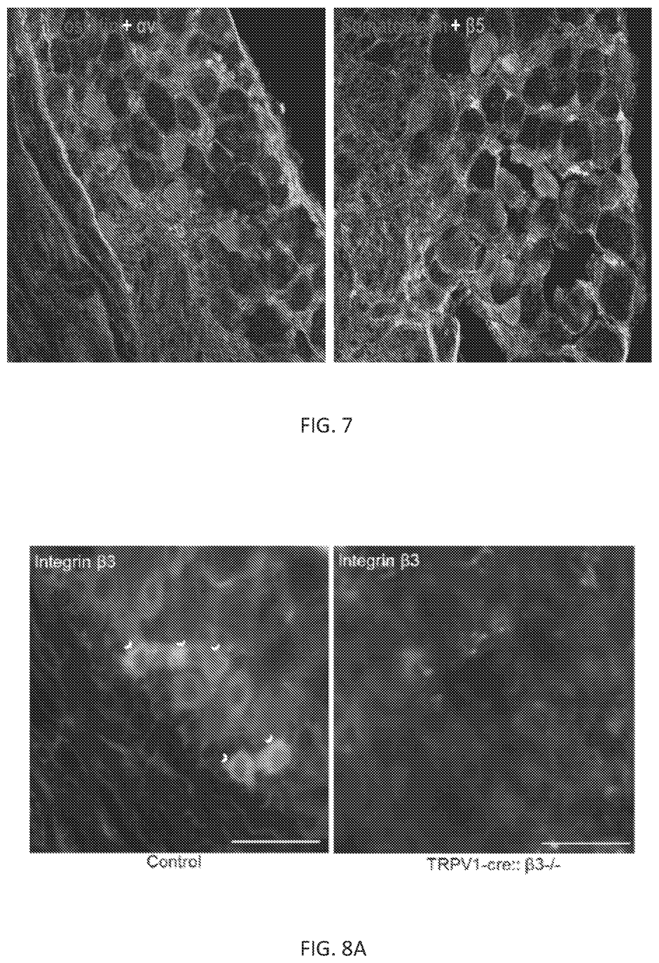

Periostin has been shown to bind to the heterodimeric αvβ3, αvβ5, and αIIbβ3 integrins (Gillan et al., 2002; Li et al., 2010; Ruan et al., 2009). Hence, the expression of αv, β3, β5, and αIIb homomers in DRG sensory neurons was investigated. Using qRT-PCR, it was found that these integrin subunits are consistently expressed in mice, dog, and monkey DRGs. See FIGS. 3A-3C. Interestingly, by using immunohistochemistry (IHC) in mice, it was found that the av and β5 subunits are expressed in almost all DRG neurons (see FIG. 7), while only a small subset of such neurons expressed the integrin β3 subunit along with the itch neurotransmitter somatostatin (SST). See FIG. 3D. As only a small percentage of DRG neurons can transmit itch, subsequent studies were focused on the αvβ3 integrin.

Recently, it was been shown that the DRG somatosensory neurons responsible for itch transmission co-express both NPPB and SST (Huang et al., 2018). To assess if these itch-specific neurons also expressed the periostin receptor of interest, SST-cre::tdTomato mice that exclusively have the red Tomato protein in SST- and NPPB-positive neurons were used. Therefore, immunohistochemistry was performed using an antibody against the β3 integrin homomer. Results confirmed the expression of β3 in SST/NPPB-expressing DRG neurons. Indeed, 93% of these SST-positive neurons were positive for β3 (135 out of 145 cells) with only 4% of the β3-positive neurons being negative for SST (6 out of 145 cells). Similarly, only 3% of SST/NPPB-positive cells were β3-negative (4 out of 145 cells), as shown in FIG. 3E. Together, these observations indicate that the integrin αvβ3 is expressed in a subset SST/NPPB-expressing DRG sensory neurons that are known to transduce inflammatory itch (Usoskin et al., 2015).

### Periostin Directly Activates Itch-Transmitting DRG Somatosensory Neurons:

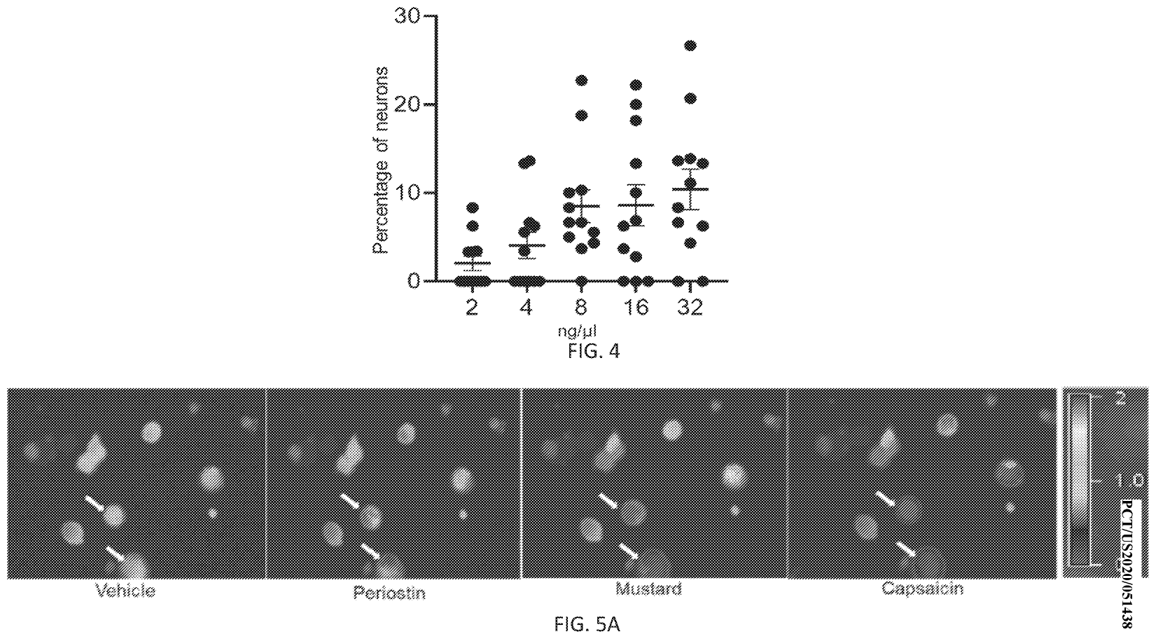

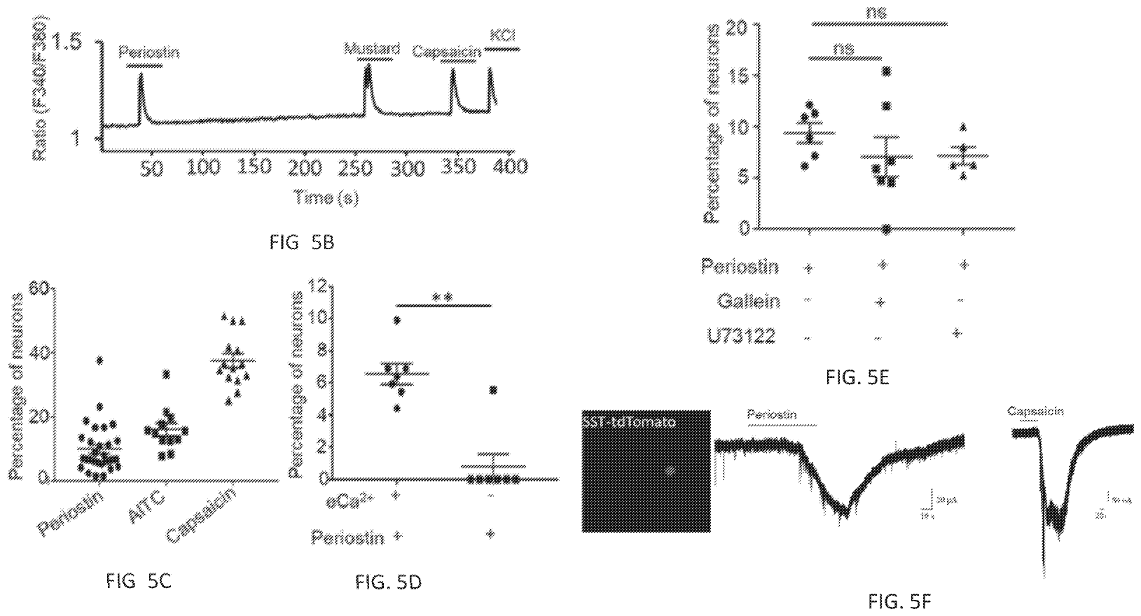

Somatosensory neurons in the DRG express receptors for the pruritogens that activate them (Han et al., 2013; Han et al., 2006; Imamachi et al., 2009). To investigate if periostin directly activated DRG sensory neurons, this cytokine was applied to cultured DRG sensory neurons loaded with the calcium chelating dye Fura-2AM. First, the response on DRG sensory neurons was measured with several different concentrations of periostin. An equal number of cells responded to periostin at 8, 16, and 32 ng/μl. See FIG. 4. Periostin at 16 ng/μl was then used throughout studies to measure calcium influx on DRG neurons. Periostin led to the entry of calcium into neurons that similarly responded to the TRPA1 agonist allyl isothiocyantate (AITC, mustard) and TRPV1 agonist capsaicin. See FIG. 5A. In parallel, an increase in amplitude in response to periostin was observed in neurons that also reacted to the TRPV1-activating capsaicin, TRPA1-activating mustard, and potassium chloride (KCl). See FIG. 5B. Periostin-dependent changes in intracellular calcium were observed in about 10±2% of DRG sensory neurons. See FIG. 5C.

Studies were conducted to determine if periostin-associated activation of DRG neurons involved the influx of extracellular or intracellular calcium. Interestingly, the removal of extracellular calcium silenced the neuronal activation induced by periostin. See FIG. 5D. Moreover, the intracellular signaling proteins PLC and/or Gβγ did not appear involved in the calcium response as the use of their respective inhibitors did not diminish the neuronal activation by periostin. See FIG. 5E. Taken together, these results indicate that extracellular calcium is involved in periostin-induced neuronal activation.

Mouse DRG neurons were isolated and cultured. The itch-transmitting neurons that expressed SST (FIG. 5F, left panel) were separated. Pilot data showed weak inward currents at 1 μg/ml; therefore, 10 μg/ml were used in a subsequent experiment. Using patch clamping, these SST-positive neurons directly responded to periostin with an inward current. See FIG. 5F, right panel. In summary, the direct activation of SST-expressing itch-transmitting neurons by periostin was shown and it was determined that this activation involved the entry of extracellular calcium to cause an inward current into the neurons that also expressed TRPV1.

Previous studies have shown that TSLP and IL-31 transduce itch signals via their respective receptors on sensory neurons (Cevikbas et al., 2014; Wilson et al., 2013). To determine the overlap of the neurons activated by these pruritogens and periostin, the ratiometric calcium response of DRG neurons to periostin, IL-31 and TSLP was assessed. The percentage of overlapping cells was calculated by counting the neurons responding to both periostin and either TSLP or IL-31 with neurons normalized to the periostin response. It was found that 16% of TSLP-responding neurons did so with periostin (35 cells TSLP/225 cells periostin) and about 38% of IL-31-activated neuron overlapped with those responding to periostin (85 cells IL-31/225 cells periostin). Taken together, these results suggest the existence of populations of neurons responding to periostin which overlap partially with those activated by the allergic pruritogenic cytokines TSLP and IL31.

The Blocking of Integrin αvβ3 on DRG Sensory Neurons Inhibits the Calcium Influx and Periostin-Induced Itch:

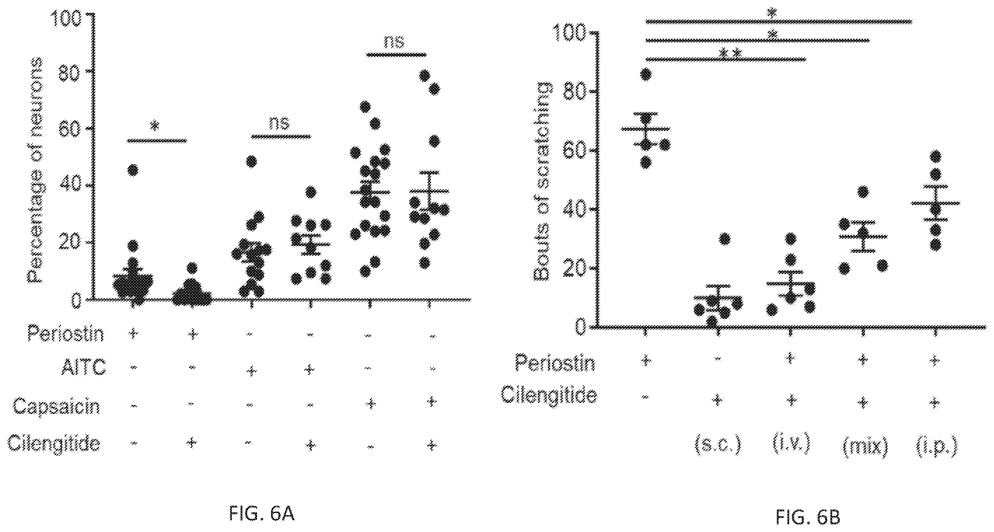

First, the effect of the broad integrin receptor antagonist cilengitide on DRG sensory neurons was examined. Cilengitide is a potent antagonist for both αvβ3 and αvβ5 with low IC₅₀'s in the nanomolar range (3 nM and 37 nM, respectively) (Goodman et al., 2002). To examine if cilengitide inhibited periostin-induced calcium responses, neurons were perfused with a buffer containing 100 nM of this antagonist. The periostin-dependent calcium response was significantly reduced during cilengitide perfusion (see FIG. 6A), thereby demonstrating that integrins are involved in such calcium influx. The perfusion with cilengitide did not reduce either AITC or capsaicin-induced calcium responses on DRG sensory neurons. See FIG. 6A. However, which of the two cilengitide-inhibited integrins was specifically involved in periostin-induced calcium response in sensory neurons was not determined by this experiment.

Next, a study was conducted to determine if the periostin-induced itch behavior in mice would also be affected by the dual integrin inhibitor cilengitide. Cilengitide was first injected into mice and it was confirmed that, alone, cilengitide did not induce itch behavior. Cilengitide was then injected intravenously (i.v.) and intraperitoneally (i.p.) 10-minutes prior to periostin intradermal injection. Finally, periostin and cilengitide were mixed together and injected subcutaneously (s.c.). Altogether, the periostin-induced itch behavior was significantly reduced when periostin was administered after or along with cilengitide by all three routes of injections. See FIG. 6B. The strongest inhibitory effect of cilengitide was seen after intravenous pre-injections of this antagonist.

### Integrin β3, TRPV1, TRPA1 and NPPB Mediate Periostin-Induced Itch:

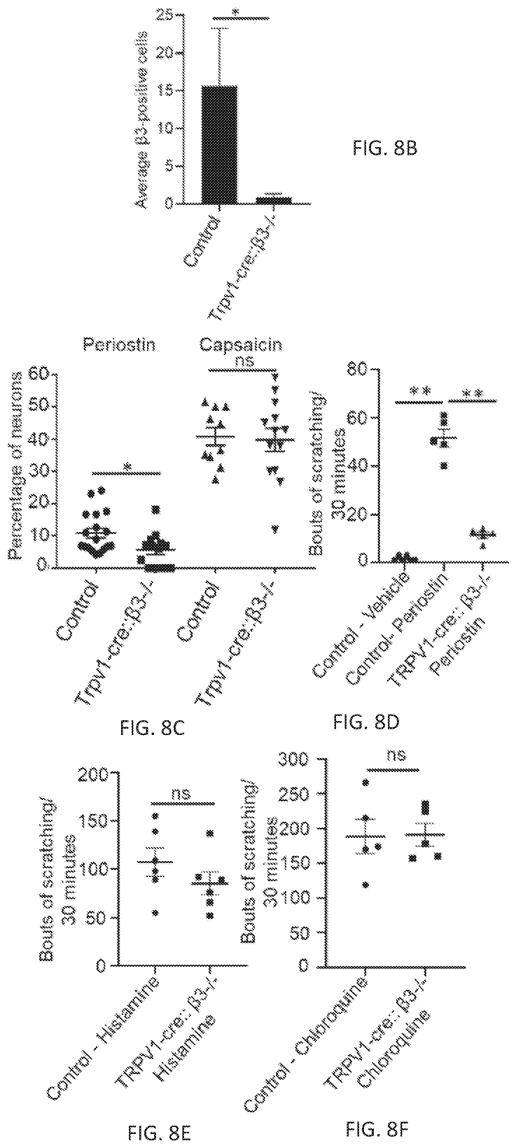

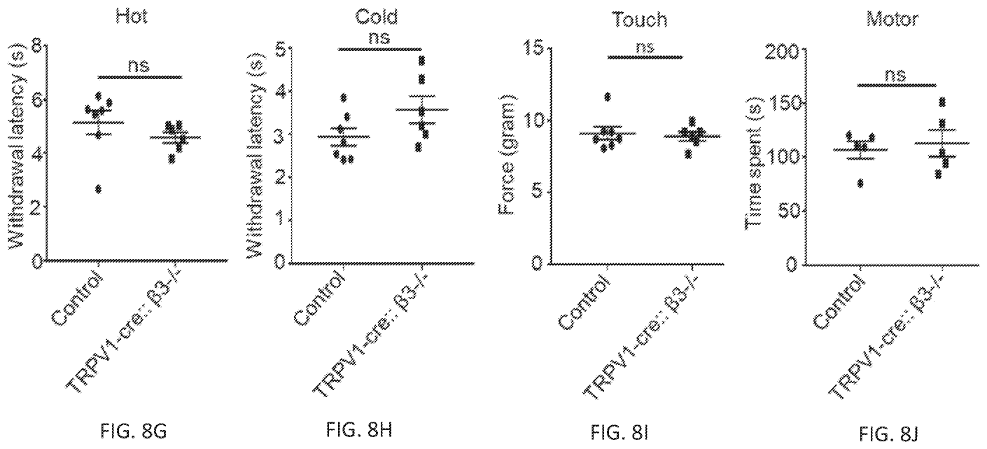

The alpha integrin subunit of the αvβ3 heterodimer is expressed in nearly all DRG neurons. See FIG. 7. As most cells responding to periostin are activated by the TRPV1 agonist capsaicin, it was suspected that the integrin αvβ3-expressing neurons are a subset of those that have TRPV1. Thus, a conditional knockout of β3 subunits was generated from a subset of TRPV1-expressing neurons by crossing a β3-flox mouse with a mouse that expresses the Cre recombinase in its TRPV1-lineage neurons (Mishra et al., 2011). It was confirmed by immunohistochemistry that the β3-subunit was knocked-out from the TRPV1-cre::β3^{-/-} mutant mice (see FIG. 8A) with an almost 95% reduction expression. See FIG. 8B. As immunostaining revealed that the β3 integrin was expressed in SST-positive itch neurons (see FIG. 3D), this integrin was conditionally knocked-out of TRPV1-expressing DRG neurons (Mishra et al., 2011). A significant reduction in the calcium response to periostin was observed in these neurons; however, no change in the calcium influx generated by capsaicin was found (see FIG. 8C) suggesting that these mice have no developmental defect in TRPV1-expressing neurons. TRPV1-cre::β3^{-/-} mice were injected with periostin (see FIG. 8D), histamine (see FIG. 8E), and chloroquine i.d. See FIG. 8F. TRPV1-cre::β3^{-/-} mice had no significant changes in histamine- and chloroquine-induced scratching bouts when compared to their control littermates. Conversely, TRPV1-cre::β3^{-/-} mice injected with periostin exhibited a significant near-complete reduction in scratching behavior when compared to their control littermates, confirming thus that the observed decrease in itch was dependent of β3. To examine if the integrin receptor β3 was also important for the pain and touch sensations, standard behavioral assays were used to measure acute pain in TRPV1-cre::β3^{-/-} and the results were compared to those of experiments done with control littermates. TRPV1-cre::β3^{-/-} mice showed no apparent differences in responses to thermal stimuli (both hot and cold), mechanosensation, and they had a normal motor function. See FIGS. 8G-8J.

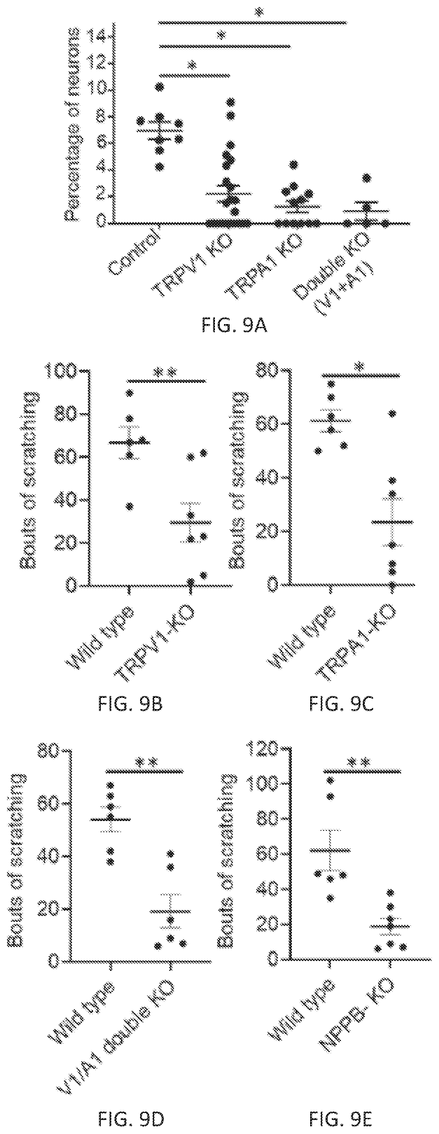

Since the neuronal calcium response induced by the histamine and chloroquine pruritogens depends on TRP-channels after activation of their respective receptors, the role of both TRPA1 and TRPV1 channels in the periostin-induced calcium response was examined. Periostin-responsive neurons overlapped with those responding to AITC (mustard) and capsaicin. See FIGS. 5A & 5B. The periostin—dependent neuronal calcium response was significantly decreased in TRPV1^{-/-}, TRPA1^{-/-}, and the decrease was highest in TRPV1^{-/-}/TRPA1^{-/-} double-knockout mice. See FIG. 9A. Altogether, these results confirm that the neurons activated by periostin utilize TRPV1 and TRPA1 synergistically. As both TRPV1 and TRPA1 ion-channels are involved in transducing itch behavior (Cevikbas et al., 2014; Imamachi et al., 2009; Lagerstrom et al., 2010; Mishra et al., 2011; Sheahan et al., 2018; Wilson et al., 2011b; Wilson et al., 2013), a study was conducted to determine if the periostin-induced itch required either one or both of these TRP channels and it was found that it was significantly diminished in TRPV1- (see FIG. 9B), TRPA1- (see FIG. 9C) single and TRPV1/TRPA1-double knockout mice. See FIG. 9D.

Finally, the itch-transmitting neuropeptide NPPB, which is expressed in a small subset of TRPV1-expressing neurons, was examined to determine if it is also involved in periostin-induced itch. As expected, a significant reduction in scratching bouts in mice deficient in NPPB compared to control animals was found. See FIG. 9E. Taken together, the results suggest the involvement of both TRPV1 and TRPA1 ion channels downstream of activated integrin αvβ3 with TRPV1 neurons releasing NPPB as a neuropeptide to transmit the pruritogenic signal to spinal cord interneurons.

### Keratinocytes Release Periostin in Response to the Cytokine Thymic Stromal Lymphopoietin:

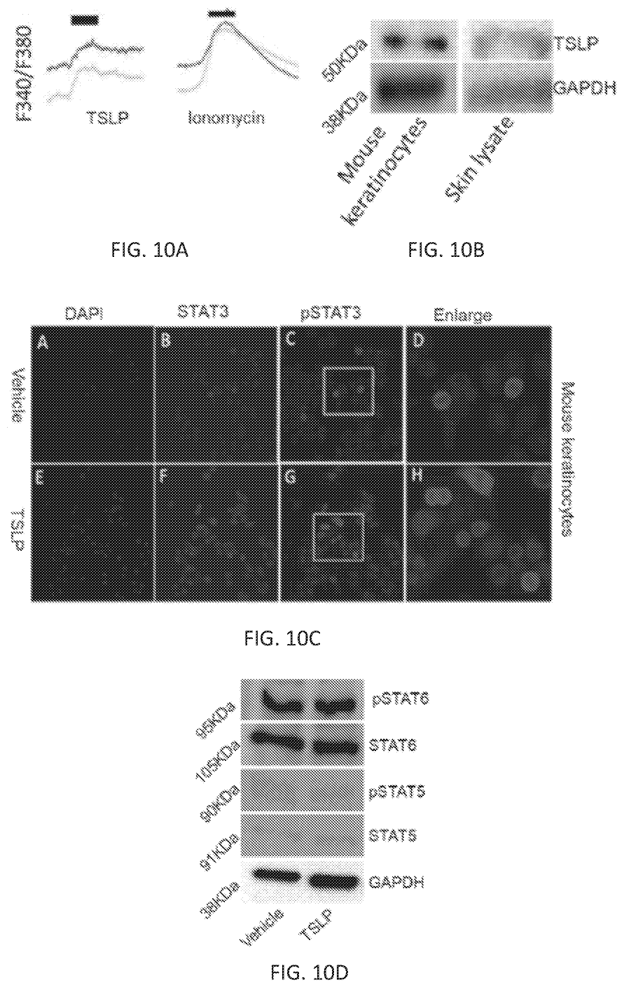

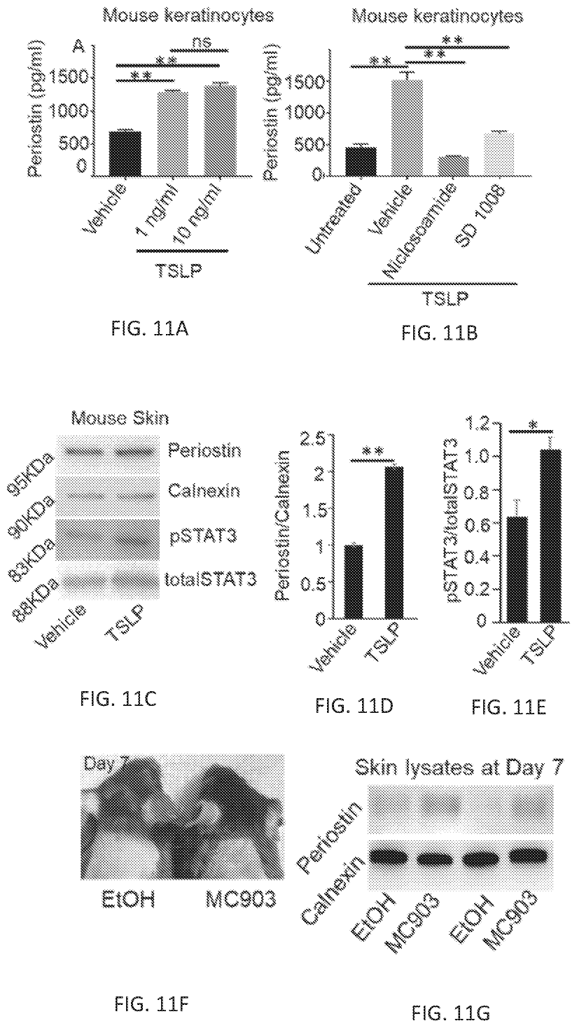

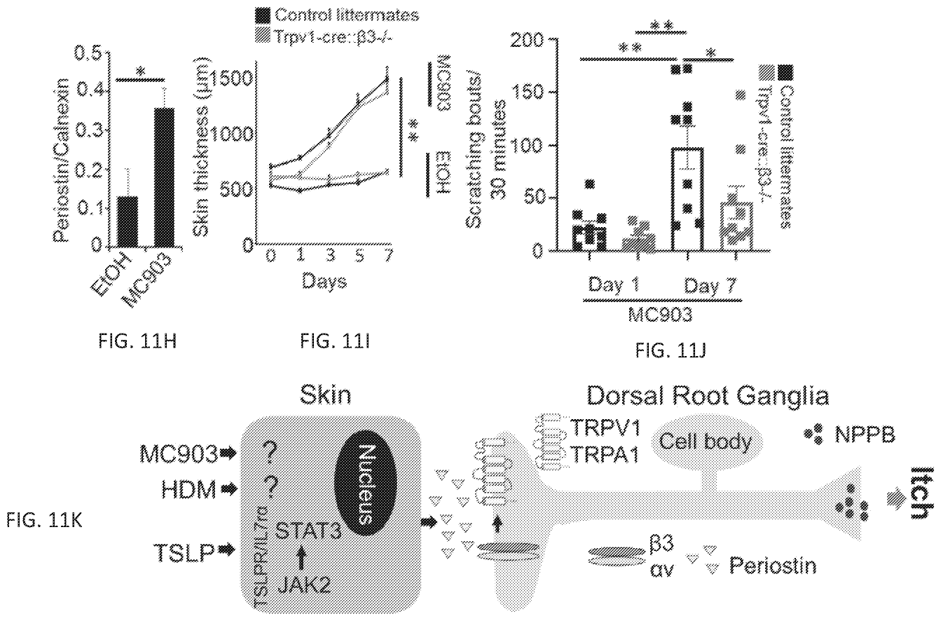

In diseases such as AD, keratinocytes contribute to the initial inflammatory response through the release of many neuro-stimulatory mediators that include the Th2 cytokine TSLP (Wilson et al., 2013). It was verified that mouse keratinocytes have functional TSLP and interleukin 7α (TSLPR/ILR7α) receptor complex (see FIG. 10A) in response to TSLP (2 ng/ml) and TSLPR protein was expressed in both mouse keratinocytes cell line and in skin lysates. See FIG. 10B. TSLP-stimulated keratinocytes induced the release of periostin in vitro. See FIG. 11A. This TSLP-induced periostin release was blocked by the JAK2 inhibitor SD 1008 and the STAT3 inhibitor niclosamide, thereby implicating the JAK/STAT pathway downstream from the TSLPR in the production and release of periostin by keratinocytes in response to TSLP. See FIG. 11B. TSLP induced both STAT3 phosphorylation and its ensuing translocation into the nucleus of mouse keratinocytes. See FIG. 10C and 10D. Surprisingly, even though STAT5 and STAT6 are involved in the IL-31 atopic itch cytokine pathway (Hermanns, 2015; Park et al., 2012), no change in either these transcription factors was detected in response to TSLP keratinocyte stimulation. See FIG. 10D. Finally, whether or not TSLP induces the release of periostin in vivo was examined. The subcutaneous injection of TSLP into the neck of mice caused a significant increase in local cutaneous levels of periostin, which was accompanied by the upregulation (activation) of phospho-STAT3, implicating the JAK/STAT pathway in TSLP-. See FIGS. 11C-11E and FIG. 13. Altogether, these results confirmed the involvement of the JAK/STAT pathway in TSLP-induced periostin production and release in vivo.

### Periostin is Involved in Chronic Allergic Itch:

Mice treated with the vitamin D3 analog calcipotriol (MC903) develop itch, skin lesions and a rise in IgE levels resembling those of humans with extrinsic AD (Moosbrugger-Martinz et al., 2017). Importantly, these changes are not dependent on mouse gender or on genetic background. MC903 was topically applied to C57BL6 mice once daily for 7 days. This application led to chronic AD-like skin changes (see FIG. 11F) and elevated skin levels of periostin. See FIGS. 11G and 11H. The topical application of MC903 to both β3-alone (control) and TRPV1-cre::β3^{-/-} mutant mice had day-dependent increase in skin thickness as compared to their control treated with vehicle (ethanol). See FIG. 11I. In contrast, MC903 applied to the dorsal neck of control mice induced significant scratching bouts that were attenuated in TRPV1-cre::β3^{-/-} mutant mice. See FIG. 11J.

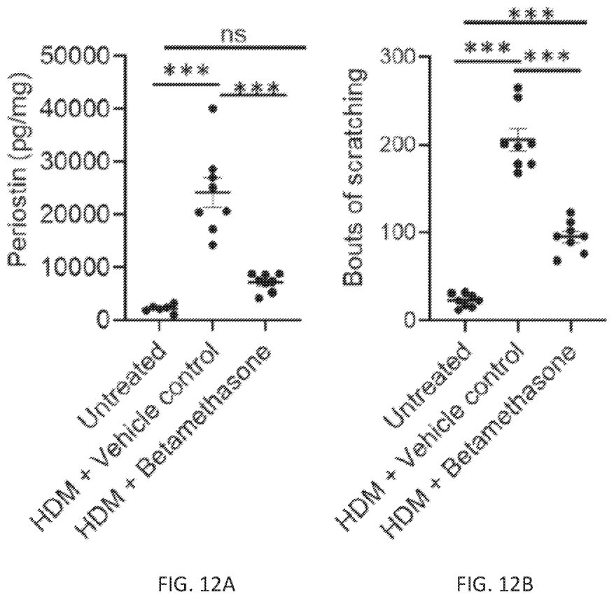

NCNgA mice were sensitized to Dermatophagoides farinae house dust mites (HDM) and then repeatedly challenged with this ubiquitous and potent allergen. The topical application of HDM on mouse dorsal skin led to elevated skin levels of periostin compared to those of mice treated with the vehicle (mineral oil). See FIG. 12A. As expected, periostin skin levels were reduced significantly after treatment with the glucocorticoid betamethasone dipropionate. See FIG. 12A. Similarly and in parallel, HDM induced robust a scratching behaviour compared to untreated controls and betamethasone dipropionate reduced such scratching. See FIG. 12B. Overall, the results suggest that periostin is one of the endogenous cutaneous pruritogens involved in the itch that develops in the MC903 and HDM chronic allergic mouse models.

In summary of the results of the studies reported above, a model of periostin-integrin αvβ3-TRPV1/TRPA1-NPPB activation is proposed that links the skin and sensory neurons and could explain the itch behavior during chronic stages of allergic skin diseases in mice, and perhaps other species. See FIG. 11K.

### Discussion:

Herein, it is shown that periostin induces itch behavior by binding to the integrin αvβ3 in a subset of itch-transmitting neurons that also express somatostatin. Evidence supports the hypothesis that periostin is an important pruritogen for chronic allergic itch. Firstly, it was established that periostin induced itch behavior in three different species (mice, dogs, and monkeys), thereby suggesting an evolutionarily conserved pathway. It was then shown that, in mice, the periostin-induced itch behavior was independent of the mast cells, T cells, B cells, and NK cells. It was then demonstrated that the integrin αvβ3 was important in the generation of itch via DRG sensory neurons with a signal propagation involving the TRPV1 and TRPA1 channels and the neuropeptide NPPB. Thirdly, it was confirmed that keratinocytes secreted periostin in response to the cytokine TSLP via the JAK/STAT pathway. Finally, the release the periostin in the skin of two mouse model of allergic skin disease was shown and it was further demonstrated that this allergic itch was dependent from the β3 integrin. Thus, for the first time, periostin-induced activation of the αvβ3 integrin in DRG sensory neurons is reported, and, without being bound to any one theory, it is proposed that the involvement of a TSLP-periostin reciprocal amplification loop that links the skin to sensory neurons to cause chronic allergic itch.

### The Periostin-Induced Itch is Mediated Through Sensory Neurons:

Atopic dermatitis is often triggered by an exposure to allergens that leads to chronic, often-severe, cutaneous inflammation and its associated itch. A wide array of mediators has been shown to be involved in the various facets of cutaneous inflammation and the allergic itch response (Cevikbas et al., 2014; Cianferoni and Spergel, 2014; Indra, 2013; Liu et al., 2016; Masuoka et al., 2012; Oetj en et al., 2017; Shang et al., 2016; Wilson et al., 2013). The fasciclin periostin is one of these chronic inflammatory mediators, but its direct stimulation of sensory neurons and its involvement in the induction of itch had not been reported earlier. Periostin, generally classified as an extracellular matrix protein, is produced by several cell types including epithelial cells and fibroblasts (Masuoka et al., 2012; Rosselli-Murai et al., 2013). It has been suggested that immune cells and parenchymal stromal cells are activated by periostin and participate in the genesis of AD skin lesions (Kim et al., 2016; Masuoka et al., 2012; Uchida et al., 2012). Periostin is highly expressed in the skin of human patients—and also dogs—with spontaneous AD (Arima et al., 2015; Izuhara et al., 2014b; Izuhara et al., 2017; Kou et al., 2014; Masuoka et al., 2012; Mineshige et al., 2018; Murota et al., 2017; Yamaguchi, 2014). The presently disclosed results using immunodeficient mice suggest that periostin directly activates sensory neurons; however, the indirect activation of other cells types (e.g., keratinocytes, fibroblasts, and dendritic cells) by periostin remains possible and can to be investigated using sophisticated genetic strategies in mice. In humans, serum levels of periostin correlate with the severity and chronicity of AD skin lesions (Kou et al., 2014). Herein, for the first time it is shown that periostin induced itch behaviors in mice, dogs, and monkeys, which suggests a direct relevance of this cytokine not only in skin lesions, but also in generation of itch.

Periostin Activates the Integrin αvβ3 on Sensory Neurons:

Integrins are transmembrane receptors that mediate cell adhesion between adjacent cells and/or the extracellular matrix (ECM). Integrins have diverse roles in several biological processes including cell migration, development, wound healing, cell differentiation, and apoptosis (Ghatak et al., 2016; Lee and Juliano, 2004). Each integrin exists as a heterodimer consisting of an α and a β subunit (Hynes, 2002). Many painful conditions have been associated with alterations in the ECM. Furthermore, integrins are present on sensory neurons that mediate inflammatory and neuropathic pain (Dina et al., 2004). The fibronectin/integrin pair participates in the upregulation of P2X4 expression after nerve injury and its subsequent neuropathic pain (Tsuda et al., 2008). The upregulation of the integrin β1 subunit in small- and medium-diameter neurons contributes to the substance P-mediated pain after mechanical injury of the capsular ligament (Zhang et al., 2017). The role of integrins in the propagation of itch and how integrins activate neuronal excitability in the DRG sensory neurons have not been previously reported.

The presently disclosed study provides new insights into the sensory biology of itch mediated via the integrin αvβ3. Again without being bound to any one theory, the role of an integrin αvβ3-mediated neuronal excitability through TRP-channels can be via two possible pathways. First, the binding of the ligand periostin to the integrin leads to neuronal signal transduction through Src-kinase that phosphorylates the TRP channels TRPV1 and TRPA1 and causes an influx of calcium that leads to the enhanced excitability of the sensory neurons and itch induction. A second hypothesis is that integrin and TRP channels are in physical contact with each other and the activation of the integrin directly leads to TRP channel activation.

Surprisingly, it was found that another receptor for periostin, the integrin αvβ5 was also expressed by almost all DRG neurons. Without being bound to any one theory, it is believed that this integrin is not relevant in itch transduction as it appears expressed on all DRG neurons, while itch-transmitting sensory neurons are a small fraction of these DRG neurons. Thus, it is conceivable that the integrin αvβ5 could play a role in cell adhesion and signaling, while αvβ3 would be involved in the generation of neuronal excitability via TRP channels. The exact role of these various integrin subunits can be further explored in the future using mice with neuron- specific deletions of single subunits. Similarly, the integrins αvβ3 and αvβ5 are also expressed in keratinocytes but the presence of functional TRPV1 and TRPA1 in these epithelial cells is the subject of controversy. While some studies support the presence of the TRPV1 and TRPA1 ion channels in keratinocytes (Ho and Lee, 2015), several other reports suggest their absence in mouse keratinocytes (Chung et al., 2003; Chung et al., 2004; Zappia et al., 2016). Further studies using keratinocyte-specific integrin-deficient mice can be used to resolve the role of keratinocytes in the periostin-associated itch in AD. Altogether, the presently disclosed findings suggest the role of the integrin αvβ3 in itch, further suggesting the use of the integrin αvβ3 as a therapeutic target in the treatment of itch associated with AD.

The Integrin αvβ3 Utilizes TRP Channels and the Neuropeptide NPPB to Transmit Periostin-Induced Itch:

Both TRPV1 and TRPA1 are generally required for the transmission of itch and pain stimuli to the CNS in rodents (Basbaum et al., 2009; Bautista et al., 2013; Julius, 2013; Julius and Basbaum, 2001; Wilson et al., 2011b). Recent studies have shown that the pro-allergic cytokine TSLP induces itch via the activation of TRPA1 (Wilson et al., 2013). Another Th2 cytokine involved in the AD-associated itch, IL-31, leads to activation of both TRPV1 and TRPA1 (Cevikbas et al., 2014). It was herein demonstrated that the extracellular matrix protein periostin, which is also relevant in the pathogenesis of chronic AD, activates both TRPV1 and TRPA1 downstream of its αvβ3 receptor, as shown for IL31. Herein, there was found an overlap between neurons responsive to periostin and those responsive to the two other pruritogenic cytokines IL31 and TSLP. Altogether, this potential overlap between the endogenous AD-relevant mediators suggests that these three cytokines could act synergistically to lead to and then perpetuate chronic allergic itch.

The neurotransmitter NPPB was recently shown to be relevant for the mechanism of IL31-associated and chemical-induced itch (Mishra and Hoon, 2013; Pitake et al., 2018). It is shown herein that the binding of periostin to the integrin αvβ3 is mediated via a TRPV1 and TRPA1-induced neuronal depolarization that results in the release of the NPPB. A recent report shows that TRPA1 is not co-localized with NPPB-expressing neurons in the DRG (Nguyen et al., 2017), which suggests the existence of a parallel release of other neurotransmitters/neuropeptides in the DRG in response to peripheral pruritogens. After release, the NPPB binds to its receptor NPRA on spinal cord interneurons, which in turn depolarizes and releases gastrin-releasing peptide (GRP) to activate GRPR-expressing interneurons before sending an itch signal to the brain (Mishra and Hoon, 2013). The behavioral results described herein implicate NPPB in the periostin-mediated itch. Although, NPPB plays a role in the signaling between DRG and the spinal cord, such signaling does not exclude the involvement of other potential neurotransmitters linked to TRPA1 channels.

### The Secretion of Periostin is Regulated by the TSLP Activation of the TSLPRJAK/STAT Pathway in Keratinocytes:

There are several molecular responses that could lead to chronic itch. The first point of contact between the skin and external/internal stimuli is the epidermis, which is made up mostly of keratinocytes. Pro-allergic stimuli activate keratinocytes to release the cytokine TSLP, a cytokine know to be involved in allergic itch, AD, asthma, and other inflammatory conditions (Cianferoni and Spergel, 2014; Indra, 2013; Straumann et al., 2001; West et al., 2012; Wilson et al., 2013). The released TSLP then binds back to keratinocytes via an autocrine/paracrine mechanism involving the TSLPR to induce the secretion periostin via the JAK/STAT3 pathway. Without being bound to any one theory, this suggests that the reciprocal activation of the pruritogenic cytokines TSLP and periostin could be at least one explanation for the chronic itch of AD. Previous studies have shown that the archetypal Th2 cytokines IL-4 and IL-13, which are uniquely important to the pathogenesis of AD, stimulate dermal fibroblasts to produce periostin and that such cytokine activate integrin-expressing keratinocytes to produce TSLP (Izuhara et al., 2014a; Masuoka et al., 2012). Herein, it is shown that TSLP also activates TSLPR-expressing keratinocytes to secrete periostin. Thus, again without being bound to any one theory, it is believed that both cytokines activate each other's secretion by keratinocytes, likely causing a reciprocal amplification loop resulting in more of each cytokine being produced over time. Importantly, a circle of amplification such as this can exist without the need for any external factor, such as AD-inducing allergens. Both TSLP (Wilson et al., 2013) and periostin (as described herein) are now shown to induce itch by directly stimulating DRG sensory neurons. TSLP, MC903, and HDM lead to an increase in the secretion of periostin that, without being bound to any one theory, could result in the paracrine release of more TSLP that would thus cause the continuous stimulation of itch-sensing DRG neurons to induce an ever-worsening itch. See FIG. 11K. This cutaneous-neuronal interaction could be one of the pathways involved in the chronic and often-severe allergic itch that is typical of AD in humans and dogs. The existence of a putative TSLP-periostin inflammation and itch-promoting reciprocal amplification loop unveils the opportunity for therapeutic interventions attempting to block this cycle. The anti-TSLP monoclonal antibody tezepelumab proved recently to have only modest effects in treating the skin lesions of itch of human AD (Simpson et al., 2017). However, interventions targeting periostin itself or its integrin receptor can be an alternative to treat atopic itch.

### Example 3

### Methods of Evalulating Itch Inhibitors

Chronic pruritus (or itch) is a problem associated with atopic dermatitis, psoriasis and other cutaneous and neurological diseases for which there are limited therapeutic options available. Herein described is a novel signaling pathway that triggers itch signaling in the skin mediated via peripheral nervous system. Integrin receptor αvβ₃ in the dorsal root ganglia (DRG) sensory neurons has been identified that acts as a key sensor to detect stimuli in the skin via endogenous mediator periostin. The data suggests that periostin/integrin signaling is one of the major pathways involved in generation of itch in mouse model of atopic dermatitis.

Two methods are used to evaluate the role of inhibitory molecules of integrin receptor. First, an in vitro assessment of inhibitors on DRG sensory neurons is used. More particularly, to determine an inhibitory action of blockers of integrin receptor that are expressed on DRG sensory neurons, calcium imaging (details below) is used. Second, an in vivo approach to determine the inhibitory effects of integrin receptor blockers is used. Here, the integrin receptor is blocked by using peptide inhibitor to test whether these blockers inhibit itch in a MC903-induced mouse model of atopic dermatitis. C57BL6 mouse DRG are used for cell culture and for the development of chronic itch atopic dermatitis mouse model. Vitamin D3 analog MC903 that has been widely known to induce atopic dermatitis like symptoms in mice is used.

A first aim of these studies is to demonstrate the inhibitory impact of a peptide inhibitor on integrin receptor using periostin as a stimulus to activate DRG neurons. A second aim is to demonstrate the inhibitory impact of a peptide inhibitor on scratching behavior in the MC903-induced mouse model of atopic dermatitis.

Integrin αvβ₃ blockers: Cilengitide and additional antagonists are used to test the inhibitory impact on integrin receptor using in vitro and in vivo assays as described below.

### Peptide Blockers:

1) Echistatin, Alpha isoform (Tocris Bioscience, Bristol, United Kingdom: catalog #3202) potent irreversible αvβ₃ integrin antagonist;

2) P11 (Tocris Bioscience, Bristol, United Kingdom: catalog #3202), Potent antagonist of αvβ3-vitronectin interaction; antiangiogenic;

3) Cilengitide (R&D Systems, Minneapolis, Minn., United States of America: catalog #5870), a potent and selective inhibitor of integrins αvβ3 and αvβ5.

The concentration of cilengitide has been already determined to block the receptor function both in vitro and in vivo, however, for echistatin and P11, concentration can be optimized via the in vitro calcium imaging assay and, based on that, the in vivo dose is calculated and a dose-dependent study is performed in a small cohort of animal before moving into a large set of mice group.

### Methods:

### In Vitro Culture and Calcium Imaging:

Cell Culture: Young 4-6 weeks old C57BL/6J mice will be obtained from The Jackson Laboratory (Ellsworth, Me., United States of America). Primary culture of DRG neurons will be performed as described (Pitake et al, 2018). DRG will be digested by 2.5 mg/ml collagenase (C7657; MilliporeSigma, Burlington, Mass., United States of America), dispersed by fire-polished Pastuer pipette and the neurons will be cultured on glass coverslips (VWR International, Radnor, Pa., United States of America) coated with 20 μl/slip of 0.4 μg/ml laminin (MilliporeSigma, Burlington, Massachusetts, United States of America) and 0.01% poly-L-lysine (MilliporeSigma, Burlington, Mass., United States of America). DRG neurons will be cultured in Dulbecco's modified Eagle's medium (DMEM, Mediatech, Inc., Manassas, Va., United States of America) containing 10% fetal bovine serum (VWR International, Radnor, Pa., United States of America), 100 units/ml penicillin and 100 μg/ml streptomycin (VWR International, Radnor, Pa., United States of America) under a condition at 37° C. in 5% CO₂. Cultured DRG neurons will be used for calcium imaging experiments within 24 hours after dissection.

Calcium Imaging: Calcium imaging will be performed on the DRG neurons as previously described (Pitake et al., 2018). Briefly, DRG neurons will be incubated for longer than 30 min in DMEM containing 1 μM of a fluorescent indicator Fura-2 AM (Enzo Life Sciences, Inc., Farmingdale, N.Y., United States of America). The neurons will be perfused in a standard bath solution containing 140 mM NaCl, 5 mM KCl, 2 mM MgCl₂, 2 mM CaCl₂, 10 mM Hepes and 10 mM D-glucose at pH 7.4 adjusted with NaOH. A calcium-free bath solution will be prepared by omitting 2 mM CaCl₂ from the standard bath solution instead of adding 5 mM EGTA. Fura-2 fluorescence excited at 340 and 380 nm and emission will be monitored at 510 nm with a digital CCD camera (Andor Clara DR-4152, Andor Technology Ltd, Belfast, United Kingdom). Data will be obtained by every 100 ms using an imaging software (NIS-Elements AR 4.13.04, Nikon Corporation, Tokyo, Japan) and analyzed by Microsoft Excel (Microsoft Corporation, Redmond, Wash., United States of America). Values of calcium responses will be normalized by dividing measured values (F) with average values of initial 5 frames (F₀) of each cell and described as F/F₀. In this study, changes in each response to an application with F/F₀>0.1 will be regarded as positive. Cells not responding to 100 mM KCl, which is applied at the end of each experiment, will be regarded as non-neuronal cells and excluded from analysis.

Periostin (10 ug/ml) will be used to evoke calcium influx. This response has been optimized. The peptide inhibitors will be tested on DRG neurons to measure calcium influx in response to periostin. Data on calcium imaging will be presented by presenting each mouse±SEM. The IC₅₀ value of the inhibitor will also be determined

### MC903 Mouse Model:

A mouse model of atopic dermatitis in mice is generated. MC903 (4 nmole) is a Vitamin D3 analog which, when applied topically to mice, elicits chronic itch behavior and is a well-established model of atopic dermatitis. Vehicle (Ethanol) will be used as a control. MC903 and vehicle is applied to the nape of the neck once daily for 7-14 days to evoke dermatitis and the scratching behaviors. At Day 10, vehicle or the compound (at least one dose) is administered intravenous/orally to the animals, and then measured the scratching behaviors for at least 60 min. In order to see the sub-chronic effect of the compound, vehicle and the compound is administered to MC903-treated animals for several days and the alteration of the scratching behaviors is measured.

### Example 4

### Methods of Treating Chronic Pruritus by Blocking Integrin Receptor

Cilengitide Inhibits Itch Response to Mediators that Induce Acute Itch and in Mouse Model of Atopic Dermatitis Associated Itch:

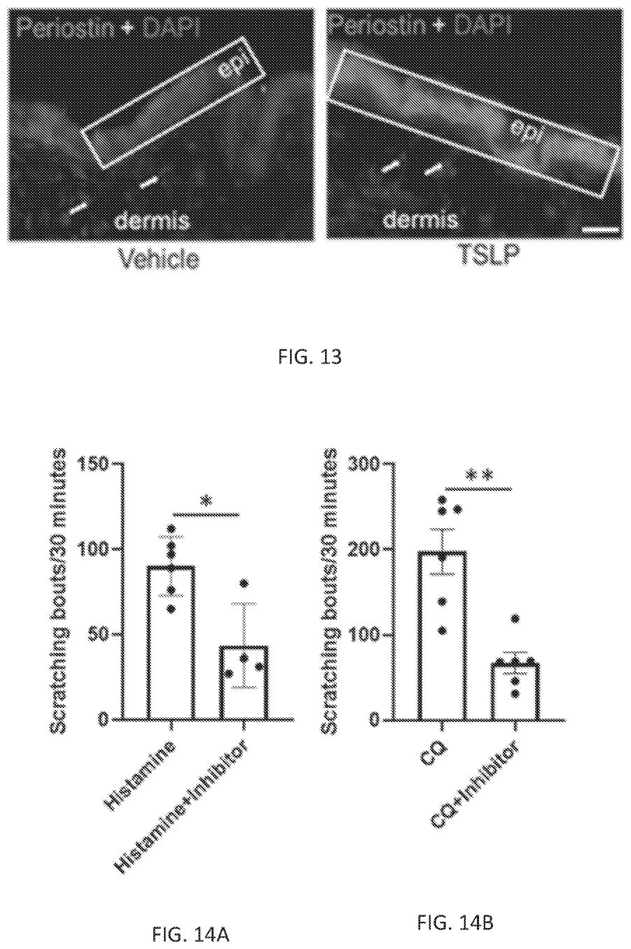

Histamine and chloroquine (CQ) classify itch into two types: histamine-dependent and histamine independent. The i.v. injection of cilengitide (100 nM, Catalog #SML1594; Millipore Sigma, Burlington, Mass., United States of America) inhibits both histamine (100 μg/10 μl) and non-histamine dependent (CQ, 100 μg/10 μl) itch (see FIGS. 14A and 14B), suggesting that cilengitide could block other mediators that are involved in acute itch and potentially act as a master inhibitor for various forms of itch.

In addition, cilengitide inhibits the chronic itch associated with atopic dermatitis. The i.v. injection of cilengitide (100 nM, Catalog #SML1594; Millipore Sigma, Burlington, Massachusetts, United States of America) inhibits MC903-induced spontaneous chronic itch at Day 10 when the itch is maximum. See FIG. 20.

### Other Inhibitors:

Additional inhibitors were studied using the calcium imaging method described in Example 3. Briefly, the Fura2-AM calcium imaging method was performed on live dorsal root ganglia (DRG) neurons (ex vivo). The DRG neurons are pre-incubated with the inhibitors P11 (Catalog #4744, Tocris Bioscience, Bristol, United Kingdom) and LM609 (Catalog #MAB1876, Millipore Sigma, Burlington, Mass., United States of America) or vehicle for 10 minutes and further stimulated with periostin or capsaicin in the presence of inhibitors. For Echistatin (Catalog #3202/100U, R&D Biosystems, Minneapolis, Minn., United States of America), MK-0429, and SB273005 (Catalog #S75-40, Selleck Chemicals Llc, Houston, Tex., United States of America), the DRG neurons were perfused with a normal Locke buffer vehicle or modified Locke buffer as described below. The values were normalized with KCl (1 mM) to identify neuronal responses in all the calcium response data below.

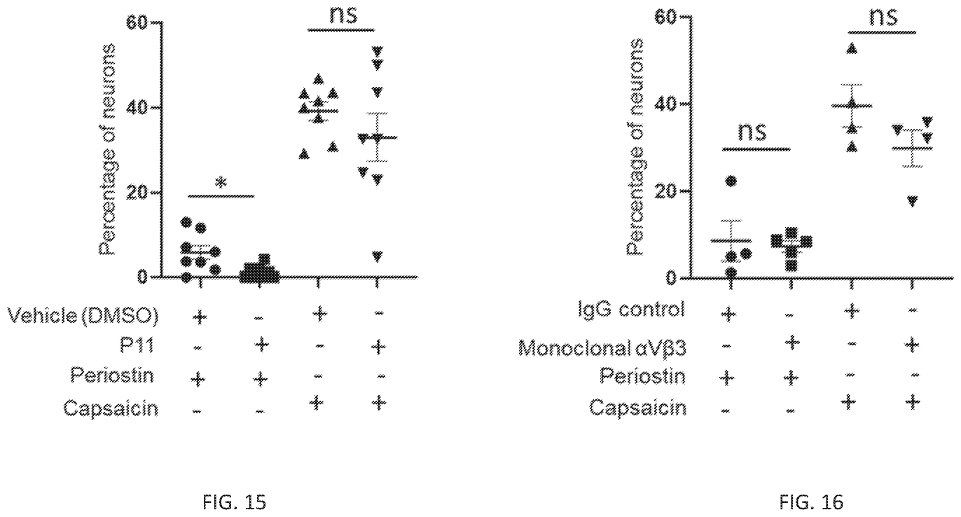

More particularly, to test the inhibitory potential of peptide inhibitor P11 against the αVβ3 receptor, P11 (50 μg/ml) was applied with periostin and capsaicin. As seen in FIG. 15, significant reduction (about 78%) in calcium influx was observed with periostin in the presence of inhibitor but not with capsaicin (an agonist for TRPV1 receptor), which suggests that P11 is an antagonist for integrin receptor. DMSO (1% (v/v)) was used as the vehicle control.

Monoclonal anti-integrin αVβ3 antibody (MAB19767) has been used to detect the inhibitory potential of the mouse integrin receptor. The inhibition of integrin was not observed when cells were stimulated with periostin in the presence of an anti-integrin αvβ₃ human antibody, LM609 (1 μg/ml). See FIG. 16. This result was not surprising, however, because LM609 does not react with mouse or rat integrin αᵥβ₃ (Mitjans et al., 1995), and thus can be viewed as a negative control in this study. LM609 has been reported as selectively inhibiting human integrin αvβ₃ (Borst et al., 2017) by binding at the interface between the β-propeller domain of the αv chain and the βI domain of the β₃ chain. LM609 binds more specifically to the ligand vitronectin so there may be a different affinity for binding to the periostin besides the species specificity. Isotype IgG antibody (1 μg/ml) was used as vehicle control in this experiment.

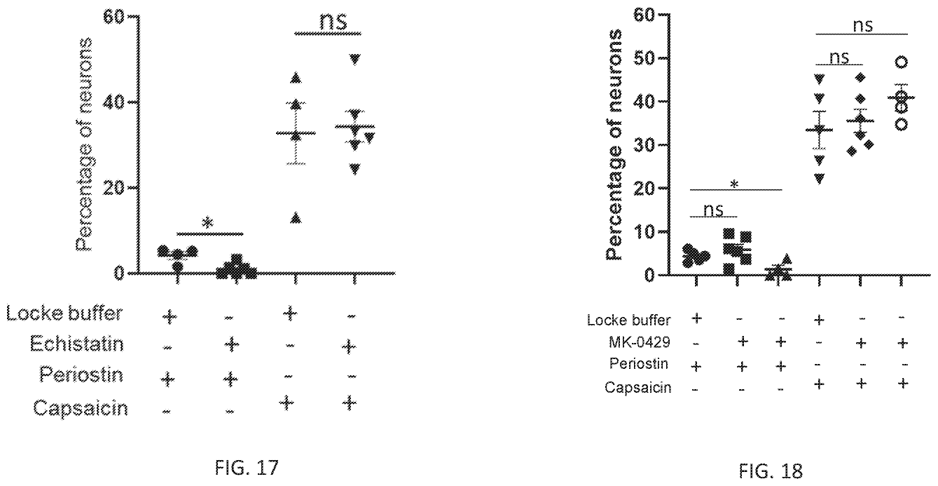

The inhibitory potential of naturally occurring peptide inhibitor Echistatin against the αvβ₃ receptor was studied. Echistatin (4 nM) was applied with periostin and capsaicin. As shown in FIG. 17, significant reduction (around 80%) in calcium influx was observed with periostin in the presence of inhibitor but not with capsaicin (an agonist for TRPV1 receptor), suggesting that Echistatin is an antagonist for integrin receptor.

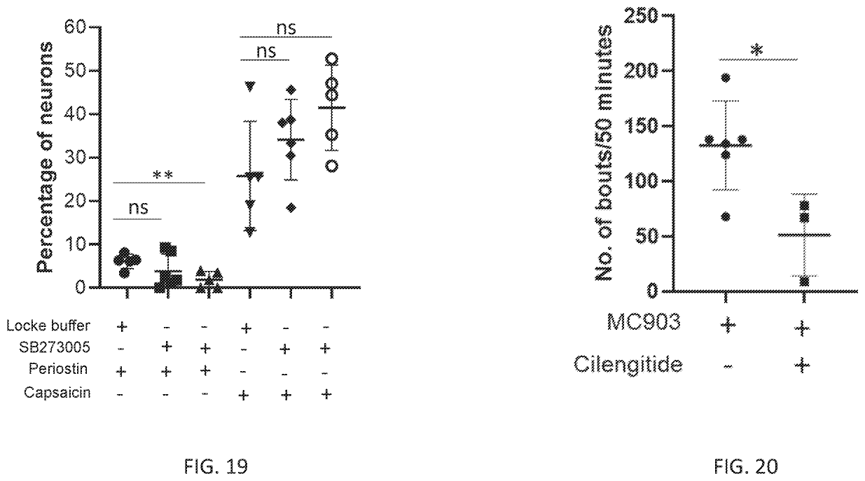

MK-0429 (also referred to as L-000845704), an inhibitor of the αvβ3 integrin (Zhou et al., 2017; Hutchingson et al., 2003), was evaluated for its potential in the prevention of calcium influx in DRG neurons. Concentrations of 80 nM or 160 nM MK-0429 antagonist were used in blocking both periostin and in testing to see if the compound has any non-specific impact on capsaicin (A TRPV1 receptor) response. Locke buffer with 0.001% DMSO (v/v) was used as the vehicle control. At the higher concentration of MK-0429, a significant reduction in periostin response was observed, but no changes were found in capsaicin response to integrin blocker MK-0439. See FIG. 18.

Additionally, SB273005, a selective inhibitor of the αvβ3 integrin (Lark et al., 2001), was evaluated for its potential in the prevention of calcium influx in DRG neurons. Concentrations of 11 nM or 20 nM of the SB273005 antagonist were used in blocking periostin and to test if the compound has any non-specific impact on capsaicin (A TRPV1 receptor) response. Locke buffer with 0.001% DMSO (v/v) was used as the vehicle control. While there was a trend in reduction (about 40%) of periostin-induced calcium response when the lower concentration (11 nM) of SB273005 was used, the reduction was not statistically significant. However, at the higher concentration (20 nM) of SB27300, significant reduction in periostin was observed. See FIG. 19. No changes were found in capsaicin response to integrin blocker SB273005. See FIG. 19.

As noted above and as seen in FIG. 20, cilengitide (100 nM) administration by tail vein injection in mouse model of atopic dermatitis (MC903) significantly reduced the spontaneous itching.

In summary, these studies show that peptide and small molecular inhibitors can block the receptor function of DRG sensory neurons and could be used as therapeutics in the treatment of both acute and chronic itch.

### REFERENCES

All references listed herein including but not limited to all patents, patent applications and publications thereof, scientific journal articles, and database entries are incorporated herein by reference in their entireties to the extent that they supplement, explain, provide a background for, or teach methodology, techniques, and/or compositions employed herein.

Abuabara, K., Yu, A. M., Okhovat, J. P., Allen, I. E., and Langan, S. M. (2018). The prevalence of atopic dermatitis beyond childhood: A systematic review and meta-analysis of longitudinal studies. Allergy 73, 696-704.

Adamson, A. S. (2017). The Economics Burden of Atopic Dermatitis. Adv Exp Med Biol 1027, 79-92.

Arima, K., Ohta, S., Takagi, A., Shiraishi, H., Masuoka, M., Ontsuka, K., Suto, H., Suzuki, S., Yamamoto, K., Ogawa, M., et al. (2015). Periostin contributes to epidermal hyperplasia in psoriasis common to atopic dermatitis. Allergol Int 64, 41-48.

Basbaum, A. I., Bautista, D. M., Scherrer, G., and Julius, D. (2009). Cellular and molecular mechanisms of pain. Cell 139, 267-284.

Bautista, D. M., Pellegrino, M., and Tsunozaki, M. (2013). TRPA1: A gatekeeper for inflammation. Annu Rev Physiol 75, 181-200.

Bautista, D. M., Wilson, S. R., and Hoon, M. A. (2014). Why we scratch an itch: the molecules, cells and circuits of itch. Nat Neurosci 17, 175-182.

Borst A J., James Z M., Zagotta W N., Ginsberg M., Rey F A., DiMaio F., Backovic M., Veesler D. (2017) The therapeutic antibody LM609 selectively inhibits ligand binding to human αvβ3 integrin via steric hindrance. Structure 25, 1732-1739.

Bosma, G. C., Custer, R. P., and Bosma, M. J. (1983). A severe combined immunodeficiency mutation in the mouse. Nature 301, 527-530.

Brenner, D. S., Golden, J. P., and Gereau, R. W. t. (2012). A novel behavioral assay for measuring cold sensation in mice. PLoS One 7, e39765.

Carstens, E. (2008). Scratching the brain to understand neuropathic itch. J Pain 9, 973-974.

Cevikbas, F., Wang, X., Akiyama, T., Kempkes, C., Savinko, T., Antal, A., Kukova, G., Buhl, T., Ikoma, A., Buddenkotte, J., et al. (2014). A sensory neuron-expressed IL-31 receptor mediates T helper cell-dependent itch: Involvement of TRPV1 and TRPA1. J Allergy Clin Immunol 133, 448-460.

Chung, M. K., Lee, H., and Caterina, M. J. (2003). Warm temperatures activate TRPV4 in mouse 308 keratinocytes. J Biol Chem 278, 32037-32046.

Chung, M. K., Lee, H., Mizuno, A., Suzuki, M., and Caterina, M. J. (2004). TRPV3 and TRPV4 mediate warmth-evoked currents in primary mouse keratinocytes. J Biol Chem 279, 21569-21575.

Cianferoni, A., and Spergel, J. (2014). The importance of TSLP in allergic disease and its role as a potential therapeutic target. Expert Rev Clin Immu 10, 1463-1474.

Cruse, G., Beaven, M. A., Ashmole, I., Bradding, P., Gilfillan, A. M., and Metcalfe, D. D. (2013). A truncated splice-variant of the FcepsilonRIbeta receptor subunit is critical for microtubule formation and degranulation in mast cells. Immunity 38, 906-917.

Dillon, S. R., Sprecher, C., Hammond, A., Bilsborough, J., Rosenfeld-Franklin, M., Presnell, S. R., Haugen, H. S., Maurer, M., Harder, B., Johnston, J., et al. (2004). Interleukin 31, a cytokine produced by activated T cells, induces dermatitis in mice. Nat Immunol 5, 752-760.

Dina, O. A., Parada, C. A., Yeh, J., Chen, X., McCarter, G. C., and Levine, J. D. (2004). Integrin signaling in inflammatory and neuropathic pain in the rat. Eur J Neurosci 19, 634-642.

Eckert, L., Gupta, S., Amand, C., Gadkari, A., Mahajan, P., and Gelfand, J. M. (2017). Impact of atopic dermatitis on health-related quality of life and productivity in adults in the United States: An analysis using the National Health and Wellness Survey. J Am Acad Dermatol 77, 274-279 e273.

Eckert, L., Gupta, S., Amand, C., Gadkari, A., Mahajan, P., and Gelfand, J. M. (2018). The burden of atopic dermatitis in US adults: Health care resource utilization data from the 2013 National Health and Wellness Survey. J Am Acad Dermatol 78, 54-61 e51.

Fukuyama, T., Ehling, S., Cook, E., and Baumer, W. (2015). Topically Administered Janus-Kinase Inhibitors Tofacitinib and Oclacitinib Display Impressive Antipruritic and Anti-Inflammatory Responses in a Model of Allergic Dermatitis. J Pharmacol Exp Ther 354, 394-405.

Fukuyama, T., Martel, B. C., Linder, K. E., Ehling, S., Ganchingco, J. R., and Baumer, W. (2018). Hypochlorous acid is antipruritic and anti-inflammatory in a mouse model of atopic dermatitis. Clin Exp Allergy 48, 78-88.

Ghatak, S., Niland, S., Schulz, J. N., Wang, F., Eble, J. A., Leitges, M., Mauch, C., Krieg, T., Zigrino, P., and Eckes, B. (2016). Role of Integrins alphalbetal and alpha2betal in Wound and Tumor Angiogenesis in Mice. Am J Pathol 186, 3011-3027.

Gillan, L., Matei, D., Fishman, D. A., Gerbin, C. S., Karlan, B. Y., and Chang, D. D. (2002). Periostin secreted by epithelial ovarian carcinoma is a ligand for alpha(V)beta(3) and alpha(V)beta(5) integrins and promotes cell motility. Cancer Res 62, 5358-5364.

Goodman, S. L., Holzemann, G., Sulyok, G. A., and Kessler, H. (2002). Nanomolar small molecule inhibitors for alphav(beta)6, alphav(beta)5, and alphav(beta)3 integrins. J Med Chem 45, 1045-1051.

Grimbaldeston, M. A., Chen, C. C., Piliponsky, A. M., Tsai, M., Tam, S. Y., and Galli, S. J. (2005). Mast cell-deficient W-sash c-kit mutant Kit W-sh/W-sh mice as a model for investigating mast cell biology in vivo. Am J Pathol 167, 835-848.

Han, L., and Dong, X. (2014). Itch mechanisms and circuits. Annu Rev Biophys 43, 331-355.

Han, L., Ma, C., Liu, Q., Weng, H. J., Cui, Y., Tang, Z., Kim, Y., Nie, H., Qu, L., Patel, K. N., et al. (2013). A subpopulation of nociceptors specifically linked to itch. Nat Neurosci 16, 174-182.

Han, S. K., Mancino, V., and Simon, M. I. (2006). Phospholipase Cbeta 3 mediates the scratching response activated by the histamine H1 receptor on C-fiber nociceptive neurons. Neuron 52, 691-703.

Hermanns, H. M. (2015). Oncostatin M and interleukin-31: Cytokines, receptors, signal transduction and physiology. Cytokine Growth Factor Rev 26, 545-558.

Ho, J. C., and Lee, C. H. (2015). TRP channels in skin: from physiological implications to clinical significances. Biophysics (Nagoya-shi) 11, 17-24.

Huang, J., Polgar, E., Solinski, H. J., Mishra, S. K., Tseng, P. Y., Iwagaki, N., Boyle, K. A., Dickie, A. C., Kriegbaum, M. C., Wildner, H., et al. (2018). Circuit dissection of the role of somatostatin in itch and pain. Nature Neuroscience 21, 707-+.

Hutchingson J H., Halczenko W., Brashear K M., Breslin M J., Coleman P J., Duong L T., et al. (2003) Nonpeptide αvβ3 antagonists. 8. In vitro and in vivo evaluation of a potent αvβ3 antagonist for the prevention and treatment of osteoporosis. J. Med. Chem. 46, 4790-4798.

Hynes, R. O. (2002). Integrins: bidirectional, allosteric signaling machines. Cell 110, 673-687.

Imamachi, N., Park, G. H., Lee, H., Anderson, D. J., Simon, M. I., Basbaum, A. I., and Han, S. K. (2009). TRPV1-expressing primary afferents generate behavioral responses to pruritogens via multiple mechanisms. Proc Natl Acad Sci USA 106, 11330-11335.

Indra, A. K. (2013). Epidermal TSLP: a trigger factor for pathogenesis of atopic dermatitis. Expert Rev Proteomics 10, 309-311.

Izuhara, K., Arima, K., Ohta, S., Suzuki, S., Inamitsu, M., and Yamamoto, K. (2014a). Periostin in Allergic Inflammation. Allergology International 63, 143-151.

Izuhara, K., Arima, K., Ohta, S., Suzuki, S., Inamitsu, M., and Yamamoto, K. I. (2014b). Periostin in Allergic Inflammation. Allergol Int 63, 143-151.

Izuhara, K., Nunomura, S., Nanri, Y., Ogawa, M., Ono, J., Mitamura, Y., and Yoshihara, T. (2017). Periostin in inflammation and allergy. Cell Mol Life Sci 74, 4293-4303.

Jensen, B. M., Swindle, E. J., Iwaki, S., and Gilfillan, A. M. (2006). Generation, isolation, and maintenance of rodent mast cells and mast cell lines. Curr Protoc Immunol Chapter 3, Unit 3 23.

Julius, D. (2013). TRP channels and pain. Annu Rev Cell Dev Biol 29, 355-384.

Julius, D., and Basbaum, A. I. (2001). Molecular mechanisms of nociception. Nature 413, 203-210.

Kardon, A. P., Polgar, E., Hachisuka, J., Snyder, L. M., Cameron, D., Savage, S., Cai, X., Karnup, S., Fan, C. R., Hemenway, G. M., etal. (2014). Dynorphin acts as a neuromodulator to inhibit itch in the dorsal horn of the spinal cord. Neuron 82, 573-586.

Kim, D. W., Kulka, M., Jo, A. R., Eun, K. M., Arizmendi, N., Tancowny, B. P., Hong, S. N., Jin, H. R., Kim, D. K., Lockey, R. F., et al. (2016). Cross-Talk Between Human Mast Cells and Epithelial Cells By IgE-Mediated Periostin Production in Eosinophilic Nasal Polyps. J Allergy Clin Immun 137, Ab186-Ab186.

Kittaka, H., and Tominaga, M. (2017). The molecular and cellular mechanisms of itch and the involvement of TRP channels in the peripheral sensory nervous system and skin. Allergol Int 66, 22-30.

Ko, M. C., and Naughton, N. N. (2000). An experimental itch model in monkeys: characterization of intrathecal morphine-induced scratching and antinociception. Anesthesiology 92, 795-805.

Kou, K., Okawa, T., Yamaguchi, Y., Ono, J., Inoue, Y., Kohno, M., Matsukura, S., Kambara, T., Ohta, S., Izuhara, K., etal. (2014). Periostin levels correlate with disease severity and chronicity in patients with atopic dermatitis. Br J Dermatol 171, 283-291.

Lagerstrom, M. C., Rogoz, K., Abrahamsen, B., Persson, E., Reinius, B., Nordenankar, K., Olund, C., Smith, C., Mendez, J. A., Chen, Z. F., etal. (2010). VGLUT2-dependent sensory neurons in the TRPV1 population regulate pain and itch. Neuron 68, 529-542.

Lark M W., Stroup G B., Dodds R A., Kapadia R., Hoffman S J., Hwang S M., et al. (2001) Antagonism of the osteoclast vitronectin receptor with an orally active nonpeptide inhibitor prevents cancellous bone loss in the ovariectomized rat. J. Bone Mineral Res. 16, 319-327.

Lee, J. W., and Juliano, R. (2004). Mitogenic signal transduction by integrin- and growth factor receptor-mediated pathways. Mol Cells 17, 188-202.

Li, G., Jin, R., Norris, R. A., Zhang, L., Yu, S., Wu, F., Markwald, R. R., Nanda, A., Conway, S. J., Smyth, S. S., et al. (2010). Periostin mediates vascular smooth muscle cell migration through the integrins alphavbeta3 and alphavbeta5 and focal adhesion kinase (FAK) pathway. Atherosclerosis 208, 358-365.

Liu, B., Tai, Y., Achanta, S., Kaelberer, M. M., Caceres, A. I., Shao, X., Fang, J., and Jordt, S. E. (2016). IL-33/ST2 signaling excites sensory neurons and mediates itch response in a mouse model of poison ivy contact allergy. Proc Natl Acad Sci USA 113, E7572-E7579.

Liu, T., and Ji, R. R. (2014). Toll-Like Receptors and Itch. In Itch: Mechanisms and Treatment, E. Carstens, and T. Akiyama, eds. (Boca Raton (FL)).

Ma, Q. (2014). Itch Modulation by VGLUT2-Dependent Glutamate Release from Somatic Sensory Neurons. In Itch: Mechanisms and Treatment, E. Carstens, and T. Akiyama, eds. (Boca Raton (Fla.)).

Madisen, L., Zwingman, T. A., Sunkin, S. M., Oh, S. W., Zariwala, H. A., Gu, H., Ng, L. L., Palmiter, R. D., Hawrylycz, M. J., Jones, A. R., et al. (2010). A robust and high-throughput Cre reporting and characterization system for the whole mouse brain. Nat Neurosci 13, 133-140.

Masuoka, M., Shiraishi, H., Ohta, S., Suzuki, S., Arima, K., Aoki, S., Toda, S., Inagaki, N., Kurihara, Y., Hayashida, S., et al. (2012). Periostin promotes chronic allergic inflammation in response to Th2 cytokines. J Clin Invest 122, 2590-2600.

Merryman-Simpson, A. E., Wood, S. H., Fretwell, N., Jones, P. G., McLaren, W. M., McEwan, N. A., Clements, D. N., Carter, S. D., Ollier, W. E., and Nuttall, T. (2008). Gene (mRNA) expression in canine atopic dermatitis: microarray analysis. Vet Dermatol 19, 59-66.

Mineshige, T., Kamiie, J., Sugahara, G., and Shirota, K. (2018). A study on periostin involvement in the pathophysiology of canine atopic skin. J Vet Med Sci 80, 103-111.

Mineshige, T., Kamiie, J., Sugahara, G., Yasuno, K., Aihara, N., Kawarai, S., Yamagishi, K., Shirota, M., and Shirota, K. (2015). Expression of Periostin in Normal, Atopic, and Nonatopic Chronically Inflamed Canine Skin. Vet Pathol 52, 1118-1126.

Mishra, S. K., and Hoon, M. A. (2010). Ablation of TrpV1 neurons reveals their selective role in thermal pain sensation. Mol Cell Neurosci 43, 157-163.

Mishra, S. K., and Hoon, M. A. (2013). The cells and circuitry for itch responses in mice. Science 340, 968-971.

Mishra, S. K., and Hoon, M. A. (2015). Transmission of pruriceptive signals. Handb Exp Pharmacol 226, 151-162.

Mishra, S. K., Tisel, S. M., Orestes, P., Bhangoo, S. K., and Hoon, M. A. (2011). TRPV1-lineage neurons are required for thermal sensation. EMBO J 30, 582-593.

Mitjans F., Sander D., Adán J., Sutter A., Martinez J M., Jäggle C S., et al. (1995) An anti-αv-integrin antibody that block integrin function inhibits the development of a human melanoma in nude mice. J. Cell Sci. 108, 2825-2835.

Mollanazar, N. K., Smith, P. K., and Yosipovitch, G. (2016). Mediators of Chronic Pruritus in Atopic Dermatitis: Getting the Itch Out? Clin Rev Allergy Immunol 51, 263-292.

Mombaerts, P., Iacomini, J., Johnson, R. S., Herrup, K., Tonegawa, S., and Papaioannou, V. E. (1992). RAG-1-deficient mice have no mature B and T lymphocytes. Cell 68, 869-877.

Moosbrugger-Martinz, V., Schmuth, M., and Dubrac, S. (2017). A Mouse Model for Atopic Dermatitis Using Topical Application of Vitamin D3 or of Its Analog MC903. Methods Mol Biol 1559, 91-106.

Morgan, E. A., Schneider, J. G., Baroni, T. E., Uluckan, O., Heller, E., Hurchla, M. A., Deng, H., Floyd, D., Berdy, A., Prior, J. L., et al. (2010). Dissection of platelet and myeloid cell defects by conditional targeting of the beta3-integrin subunit. FASEB J 24, 1117-1127.

Murota, H., Yang, L. L., and Katayama, I. (2017). Periostin in the pathogenesis of skin diseases. Cell Mol Life Sci 74, 4321-4328.

Nguyen, M. Q., Wu, Y., Bonilla, L. S., von Buchholtz, L. J., and Ryba, N. J. P. (2017). Diversity amongst trigeminal neurons revealed by high throughput single cell sequencing. PLoS One 12, e0185543.

Oaklander, A. L. (2011). Neuropathic itch. Semin Cutan Med Surg 30, 87-92.

Odhiambo, J. A., Williams, H. C., Clayton, T. O., Robertson, C. F., Asher, M. I., and Group, I. P. T. S. (2009). Global variations in prevalence of eczema symptoms in children from ISAAC Phase Three. J Allergy Clin Immunol 124, 1251-1258 e1223.

Oetjen, L. K., Mack, M. R., Feng, J., Whelan, T. M., Niu, H., Guo, C. J., Chen, S., Trier, A. M., Xu, A. Z., Tripathi, S. V., et al. (2017). Sensory Neurons Co-opt Classical Immune Signaling Pathways to Mediate Chronic Itch. Cell 171, 217-228 e213.

Olivry, T., and Baumer, W. (2015). Atopic itch in dogs: pharmacology and modeling. Handb Exp Pharmacol 226, 357-369.

Olivry, T., Mayhew, D., Paps, J. S., Linder, K. E., Peredo, C., Rajpal, D., Hofland, H., and Cote-Sierra, J. (2016). Early Activation of Th2/Th22 Inflammatory and Pruritogenic Pathways in Acute Canine Atopic Dermatitis Skin Lesions. J Invest Dermatol 136, 1961-1969.

Paps, J. S., Baumer, W., and Olivry, T. (2016). Development of an Allergen-induced Atopic Itch Model in Dogs: A Preliminary Report. Acta Derm Venereol 96, 400-401.

Park, K., Park, J. H., Yang, W. J., Lee, J. J., Song, M. J., and Kim, H. P. (2012). Transcriptional activation of the IL31 gene by NFAT and STATE. J Leukoc Biol 91, 245-257.

Paus, R., Schmelz, M., Biro, T., and Steinhoff, M. (2006). Frontiers in pruritus research: scratching the brain for more effective itch therapy. J Clin Invest 116, 1174-1186.

Pitake, S., Ralph, P. C., DeBrecht, J., and Mishra, S. K. (2018). Atopic Dermatitis Linked Cytokine Interleukin-31 Induced Itch Mediated via a Neuropeptide Natriuretic Polypeptide B. Acta Derm Venereol 98, 795-796.

Pogorzala, L. A., Mishra, S. K., and Hoon, M. A. (2013). The cellular code for mammalian thermosensation. J Neurosci 33, 5533-5541.

Rosselli-Murai, L. K., Almeida, L. O., Zagni, C., Galindo-Moreno, P., Padial-Molina, M., Volk, S. L., Murai, M. J., Rios, H. F., Squarize, C. H., and Castilho, R. M. (2013). Periostin responds to mechanical stress and tension by activating the MTOR signaling pathway. PLoS One 8, e83580.

Ruan, K., Bao, S., and Ouyang, G. (2009). The multifaceted role of periostin in tumorigenesis. Cell Mol Life Sci 66, 2219-2230.

Shahwan, K. T., and Kimball, A. B. (2017). Itch intensity in moderate-to-severe plaque psoriasis versus atopic dermatitis: A meta-analysis. J Am Acad Dermatol 76, 1198-1200 e1191.

Shang, H., Cao, X. L., Wan, Y. J., Meng, J., and Guo, L. H. (2016). IL-4 Gene Polymorphism May Contribute to an Increased Risk of Atopic Dermatitis in Children. Dis Markers.

Sheahan, T. D., Hachisuka, J., and Ross, S. E. (2018). Small RNAs, but Sizable Itch: TRPA1 Activation by an Extracellular MicroRNA. Neuron 99, 421-422.

Shim, W. S., Tak, M. H., Lee, M. H., Kim, M., Kim, M., Koo, J. Y., Lee, C. H., Kim, M., and Oh, U. (2007). TRPV1 mediates histamine-induced itching via the activation of phospholipase A2 and 12-lipoxygenase. J Neurosci 27, 2331-2337.

Shimada, S. G., and LaMotte, R. H. (2008). Behavioral differentiation between itch and pain in mouse. Pain 139, 681-687.

Shiraishi, H., Masuoka, M., Ohta, S., Suzuki, S., Arima, K., Taniguchi, K., Aoki, S., Toda, S., Yoshimoto, T., Inagaki, N., et al. (2012). Periostin contributes to the pathogenesis of atopic dermatitis by inducing TSLP production from keratinocytes. Allergol Int 61, 563-572.

Shultz, L. D., Schweitzer, P. A., Christianson, S. W., Gott, B., Schweitzer, I. B., Tennent, B., McKenna, S., Mobraaten, L., Rajan, T. V., Greiner, D. L., et al. (1995). Multiple defects in innate and adaptive immunologic function in NOD/LtSz-scid mice. J Immunol 154, 180-191.

Simpson, E. L., Bruin-Weller, M., Flohr, C., Ardern-Jones, M. R., Barbarot, S., Deleuran, M., Bieber, T., Vestergaard, C., Brown, S. J., Cork, M. J., et al. (2017). When does atopic dermatitis warrant systemic therapy? Recommendations from an expert panel of the International Eczema Council. J Am Acad Dermatol 77, 623-633.

Storan, E. R., O'Gorman, S. M., McDonald, I. D., and Steinhoff, M. (2015). Role of cytokines and chemokines in itch. Handb Exp Pharmacol 226, 163-176.

Straumann, A., Bauer, M., Fischer, B., Blaser, K., and Simon, H. U. (2001). Idiopathic eosinophilic esophagitis is associated with a T(H)2-type allergic inflammatory response. J Allergy Clin Immun 108, 954-961.

Takahashi, N., Sugaya, M., Suga, H., Oka, T., Kawaguchi, M., Miyagaki, T., Fujita, H., and Sato, S. (2016). Thymic Stromal Chemokine TSLP Acts through Th2 Cytokine Production to Induce Cutaneous T-cell Lymphoma. Cancer Res 76, 6241-6252.

Tsuda, M., Toyomitsu, E., Komatsu, T., Masuda, T., Kunifusa, E., Nasu-Tada, K., Koizumi, S., Yamamoto, K., Ando, J., and Inoue, K. (2008). Fibronectin/integrin system is involved in P2X(4) receptor upregulation in the spinal cord and neuropathic pain after nerve injury. Glia 56, 579-585.

Uchida, M., Shiraishi, H., Ohta, S., Arima, K., Taniguchi, K., Suzuki, S., Okamoto, M., Ahlfeld, S. K., Ohshima, K., Kato, S., et al. (2012). Periostin, a matricellular protein, plays a role in the induction of chemokines in pulmonary fibrosis. Am J Respir Cell Mol Biot 46, 677-686.

Usoskin, D., Furlan, A., Islam, S., Abdo, H., Lonnerberg, P., Lou, D., Hjerling-Leffler, J., Haeggstrom, J., Kharchenko, O., Kharchenko, P. V., et al. (2015). Unbiased classification of sensory neuron types by large-scale single-cell RNA sequencing. Nat Neurosci 18, 145-153.

Voisin, T., Bouvier, A., and Chiu, I. M. (2017). Neuro-immune interactions in allergic diseases: novel targets for therapeutics. Int Immunol 29, 247-261.

Weidinger, S., and Novak, N. (2016). Atopic dermatitis. Lancet 387, 1109-1122.

West, E. E., Kashyap, M., and Leonard, W. J. (2012). TSLP: A Key Regulator of Asthma Pathogenesis. Drug Discov Today Dis Mech 9.

Wilson, S. R., Gerhold, K. A., Bifolck-Fisher, A., Liu, Q., Patel, K. N., Dong, X., and Bautista, D. M. (2011a). TRPA1 is required for histamine-independent, Mas-related G protein-coupled receptor-mediated itch. Nat Neurosci 14, 595-602.

Wilson, S. R., Gerhold, K. A., Bifolck-Fisher, A., Liu, Q., Patel, K. N., Dong, X. Z., and Bautista, D. M. (2011b). TRPA1 is required for histamine-independent, Mas-related G protein-coupled receptor-mediated itch. Nature Neuroscience 14, 595-U582.

Wilson, S. R., The, L., Batia, L. M., Beattie, K., Katibah, G. E., McClain, S. P., Pellegrino, M., Estandian, D. M., and Bautista, D. M. (2013). The epithelial cell-derived atopic dermatitis cytokine TSLP activates neurons to induce itch. Cell 155, 285-295.

Yamaguchi, Y. (2014). Periostin in Skin Tissue Skin-Related Diseases. Allergol Int 63, 161-170.

Yosipovitch, G., and Samuel, L.S. (2008). Neuropathic and psychogenic itch. Dermatol Ther 21, 32-41.

Zappia, K. J., Garrison, S. R., Palygin, O., Weyer, A. D., Barabas, M. E., Lawlor, M. W., Staruschenko, A., and Stucky, C. L. (2016). Mechanosensory and ATP Release Deficits following Keratin14-Cre-Mediated TRPA1 Deletion Despite Absence of TRPA1 in Murine Keratinocytes. PLoS One 11, e0151602.

Zhang, S., Zhao, E., and Winkelstein, B. A. (2017). A Nociceptive Role for Integrin Signaling in Pain After Mechanical Injury to the Spinal Facet Capsular Ligament. Ann Biomed Eng 45, 2813-2825.

Zhou X., Zhang J., Haimbach R., Zhu W., Mayer-Ezell R., Garcia-Calvo M., et al. (2017) An integrin antagonist (MK-0429) decreases proteinuria and renal fibrosis in the ZSF1 rat diabetic nephropathy model. Pharmacol. Res. Perspec. 5, e00354.

It will be understood that various details of the presently disclosed subject matter can be changed without departing from the scope of the presently disclosed subject matter. Furthermore, the foregoing description is for the purpose of illustration only, and not for the purpose of limitation.

What is claimed is:

1. A method of treating or alleviating pruritus, optionally chronic pruritus, in a subject in need of treatment thereof, the method comprising administering to the subject an effective amount of an antagonist of integrin αᵥβ₃.

2. The method of claim 1, wherein the pruritis is associated with one of atopic dermatitis or psoriasis.

3. The method of claim 1 or claim 2, wherein administration of the antagonist blocks periostin-integrin signaling.

4. The method of any one of claims 1-3, wherein the antagonist has a 50% inhibitory concentration (IC₅₀) for integrin αᵥβ₃ of about 50 nanomolar (nM) or less, optionally about 10 nM or less.

5. The method of any one of claims 1-4, wherein the antagonist is selective for integrin αᵥβ₃ compared to integrin αᵥβ₅.

6. The method of claim 5, wherein the antagonist has a 50% inhibitor concentration (IC₅₀) for integrin αᵥβ₃ that is at least about 2 times lower than the antagonist's IC₅₀ for integrin αᵥβ₅, optionally at least about 5 times lower.

7. The method of any one of claims 1-6, wherein the antagonist of integrin αᵥβ₃ is selected from the group consisting of an antibody or a fragment thereof, a peptide comprising an RGD sequence, a peptide comprising an SDV sequence, a peptidomimetic, an amine salt, a phosphoric acid salt, and a small molecule antagonist of integrin αᵥβ₃.

8. The method of claim 7, wherein the antagonist of integrin αᵥβ₃ is a peptide comprising an RGD sequence.

9. The method of claim 8, wherein the peptide comprising an RGD sequence is a synthetic peptide.

10. The method of claim 9, wherein the synthetic peptide is a cyclic peptide and/or a tetra- or pentapeptide.

11. The method of claim 9 or claim 10, wherein in addition to the RGD sequence the synthetic peptide comprises a residue based on a D-amino acid and/or a N-methylated residue.

12. The method of claim 11, wherein the antagonist is cilengitide.

13. The method of claim 8, wherein the peptide comprising an RGD sequence is a naturally occurring peptide.

14. The method of claim 13, wherein the peptide comprising an RGD sequence is a disintegrin.

15. The method of claim 14, wherein the disintegrin is Echistatin.

16. The method of claim 7, wherein the antagonist is a peptide that comprises a SDV sequence.

17. The method of claim 16, wherein the peptide is His-Ser-Asp-Val-His-Lys-NH₂ (SEQ ID NO: 2, P11).

18. The method of claim 7, wherein the antagonist is a peptidomimetic, wherein said peptidomimetic is a peptidomimetic of a peptide comprising an RGD sequence, optionally wherein said peptidomimetic comprises a monocyclic central phenyl ring, a monocyclic central heterocyclic ring, a bicyclic central ring, or an acyclic backbone.

19. The method of claim 7, wherein the antagonist is a small molecule antagonist of integrin αᵥβ₃, optionally wherein the antagonist is (S)-3-(6-methoxypyridin-3-yl)-3-(2-oxo-3-(3-(5,6,7,8-tetrahydro-1,8-naphthyridin-2-yl)propyl)imidazolidin-1-yl)propanoic acid (L000845704) or (4S)-2,3,4,5-tetrahydro-8-[2-[6-(methylamino)-2-pyridinyl]ethyoxy]-3-oxo-2-(2,2,2-trifluoroethyl)-1H-2-benzazepine-4-acetic acid (SB273005).

20. A method of treating or alleviating pruritus, optionally chronic or acute pruritus, in a subject in need of treatment thereof, the method comprising administering to the subject an effective amount of cilengitide.


SEQUENCE LISTING (2 sequences)

SEQ ID NO: 1 - protein, 5 aa, Artificial sequence
  synthetic sequence
  misc_feature (1)..(5): cyclic
  MOD_RES (4)..(4): D-Phe
  MOD_RES (4)..(5): N-Methyl
RGDXV

SEQ ID NO: 2 - protein, 6 aa, Artificial sequence
  synthetic sequence
  misc_feature (6)..(6): C terminal NH2
HSDVHK